(19) United States
(12) Patent Application Publication
(10) Pub. No.: US 2008/0064717 A1
(43) Pub. Date: Mar. 13, 2008

(54) Inhibitors of diacylglycerol O-acyltransferase type 1 enzyme

(76) Inventors: Rajesh Iyengar, Lindenhurst, IL (US); Gang Zhao, Northbrook, IL (US); Jennifer Freeman, Grayslake, IL (US); Ju Gao, Gurnee, IL (US); Andrew S. Judd, Grayslake, IL (US); Philip R. Kym, Libertyville, IL (US); John Lynch, Kenosha, WI (US); Mathew Mulhern, Lake Villa, IL (US); Andrew Souers, Evanston, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/749,952

(22) Filed: May 17, 2007

Related U.S. Application Data
(60) Provisional application No. 60/801,890, filed on May 19, 2006.
(60) Provisional application No. 60/871,043, filed on Dec. 20, 2006.

(51) Int. Cl. A61K 31/4402; A61K 31/17; A61K 31/19; A61P 3/00; C07C 61/06; C07D 471/04; C07D 213/46; C07C 275/28; A61P 1/16; A61K 31/4375

(52) U.S. Cl. 514/300; 514/354; 514/573; 514/596; 546/122; 546/328; 562/503; 564/55

(57) ABSTRACT

The present invention relates to compounds of formula (I): 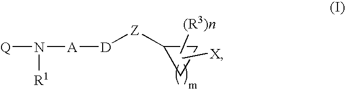
wherein R¹, R³, X, Q, Z, A, 9, m, and n are defined herein Pharmaceutical compositions and methods for treating DGAT-1 related diseases or conditions are also disclosed.

### CROSS REFERENCE TO RELATED APPLICATIONS

The present application claims the benefit of U.S. Provisional Application No. 60/801,890, filed on May 19, 2006, and U.S. Provisional Application No. 60/871,043, filed Dec. 20, 2006, both of which are hereby incorporated by reference in their entirety.

### FIELD OF THE INVENTION

Compounds that are inhibitors of the diacylglycerol O-acyltransferase type 1 (DGAT-1) enzyme are disclosed. Methods of using such compounds to inhibit the activity of diacylglycerol O-acyltransferase type 1 and pharmaceutical compositions including such compounds are also encompassed

### BACKGROUND OF THE INVENTION

Triacylglycerides represent the major form of energy storage in eukaryotes, and disorders or imbalance in triacylglycerides metabolism are implicated in the pathogenesis and the increased risk for obesity, insulin resistance, type II diabetes, nonalcoholic fatty liver disease and coronary heart disease (Lewis, et al., Endocrine Reviews 23:201, 2002) Storage of excess triacylglycerides in lean tissues, such as liver, muscle, and other peripheral tissues, leads to lipid-induced dysfunction in those tissues; thus, reducing fat accumulation in nonadipose sites appears to be of benefit in the treatment of lipotoxicity (Unger, R. H. Endocrinology, 144: 5159-5165, 2003). Accumulation of excess triacylglycerides in white adipose tissue (WAT) leads to obesity, a condition that is associated with decreased life span, type II diabetes, coronary artery disease, hypertension, stroke, and the development of some cancers (Grundy, S. M. Endocrine 13(2): 155-165, 2000). Obesity is a chronic disease that is highly prevalent in modern society and current pharmacological treatment options are limited, creating a need to develop pharmaceutical agents for the treatment of obesity that are safe and effective.

Diacylglycerol O-acyltransfereases (DGATs) are membrane-bound enzymes that catalyze the terminal step of triacylglycerides biosynthesis. Two enzymes that display DGAT activity have been characterized: DGAT-1 (diacylglycerol O-acyltransferase type 1) (U.S. Pat. No. 6,100,077; Cases, et al., Proc. Nat. Acad. Sci. 95:13018-13023, 1998) and DGAT-2 (diacylglyerol O-acyltransferase type 2) (Cases, et al., J. Biol. Chem. 276:38870-38876, 2001) DGAT-1 and DGAT-2 share only 12% sequence identity. Significantly, DGAT-1 null mice are resistant to diet-induced obesity and have increased sensitivity to insulin and leptin (Smith, et al., Nature Genetics 25:87-90, 2000; Chen and Farese, Trends Cardiovasc Med. 10:188, 2000; Chen et al., J. Clin. Invest. 109:10049, 2002). DGAT-1 deficient mice are protected against hepatic steatosis, demonstrate increased energy expenditure, and decreased levels of tissue triacylglycerides. In addition to improved triacylglycerides metabolism, DGAT-1 deficient mice also have improved glucose metabolism, with lower glucose and insulin levels following a glucose load, in comparison to wild-type mice. Partial DGAT-1 deficiency in heterozygous DGAT-1±animals is sufficient to deliver an intermediate phenotype on body weight, adiposity, and insulin and glucose metabolism when compared to wild type and homozygous littermates (Chen and Farese, Arterioscler, Thromb. Vasc. Biol. 25:482-486, 2005), and small molecule DGAT-1 inhibitors have been reported to induce weight loss in diet-induced obese (DIO) mice (US 2004/0224997) The phenotypes of DGAT-1 deficient mice, and the pharmacological activity reported with DGAT-1 inhibitors suggests that the discovery of small molecules that effectively block the conversion of diacylglycerol to triacylglycerides by inhibiting the DGAT-1 enzyme can have utility in the treatment of obesity and other diseases associated with triacylglycerides imbalance.

### SUMMARY OF THE INVENTION

One aspect of the invention is directed towards a compound of formula (I), or a pharmaceutically acceptable salt, prodrug, salt of a prodrug, or a combination thereof, 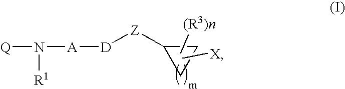
wherein

Q is —C(═Y)N(R²)(R^{2a}), —C(═W)(R^{b}), —R^{b}, —S(O)₂(R^{b}), or —C(O)O(R^{b});

R¹ and R^{2a}, are each independently hydrogen or lower alkyl;

R² is alkyl, aryl, heteroaryl, cycloalkyl, cycloalkenyl, or heterocycle; wherein the aryl, heteroaryl, cycloalkyl, cycloalkenyl, and heterocycle are each independently further unsubstituted or substituted with 1, 2, 3, 4, or 5 substituents independently selected from the group consisting of alkyl, alkenyl, alkynyl, nitro, —CN, halogen, ethylenedioxy, methlylenedioxy, haloalkyl, —OR^{a}, —O—C(O)(R^{a}), —S(R^{a}), —S(O)(R^{b}), —S(O)₂(R^{b}), —C((O)(R^{a}), —C(O)O(R^{a}), —N(R^{a})₂, —N(R^{a})—C(O)(R^{a}), —C(O)N(R^{a})₂, —S(O)₂N(R^{a})₂, R⁴, —(CR^{c}R^{d})ₜ, —OR^{a}, —(CR^{c}R^{d})ₜ—O—C(O)(R^{a}), —(CR^{c}R^{d})ₜ—S(R^{a}), —(CR^{c}R_{d})ₜ—S(O)(R^{b}), —(CR^{c}R^{d})ₜ—S(O)₂(R^{b}), —(CR^{c}R^{d})ₜ—C(O)(R^{a}), —(CR^{c}R^{d})ₜ—C(O)O(R^{a}), —(CR^{c}R^{d})ₜ—N(R^{a})₂₃, —(CR^{c}R^{d})ₜ—N(R^{a})—C(O)(R^{a}), —(CR^{c}R^{d})ₜ—C(O)N(R^{a})₂, —(CR^{c}R^{d})ₜ—S(O)₂N(R^{a})₂, and —(CR^{c}R^{d})ₜ—R⁴;

R³ represents a substituent group selected from the group consisting of alkyl, haloalkyl, —OR^{a}, and halogen,

m is 1, 2, 3, 4, or 5;

n is 0, 1, or 2;

A and D are each a monocyclic ring selected from the group consisting of phenyl, heteroaryl, cycloalkyl, and cycloalkenyl; each of which is optionally further substituted with 1, 2, 3, 4, or 5 substituents as represented by T, wherein each T is independently selected from the group consisting of alkyl, alkenyl, alkynyl, nitro, —CN, halogen, ethylenedioxy, methylenedioxy, haloalkyl, —OR^{c}, —O—C(O)(R^{e}), —S(R^{e}), —S(O)(R^{f}), —S(O)₂(R^{f}), —C(O)(R^{e}), —C(O)O(R^{e}), —N(R^{e})₂, —N(R^{e})—C(O)(R^{e}), —C(O)N(R^{e})₂, —S(O)₂N(R)ₑ)₂, —(CR^{c}R^{d})ₜ—OR^{e}, —(CR^{c}R^{d})ₜ—O—C(O)(R^{e}), —(CR^{c}R^{d})ₜ—S(R^{e}), —(CR^{c}R^{d})ₜ—S(O)(R^{f}), —(CR^{c}R^{d})ₜ—S(O)₂(R^{f}), —(CR^{c}R^{d})ₜ—C(O)(R^{e}), —(CR^{c}R^{d})ₜ—C(O)O(R^{e}), —(CR^{c}R^{d})ₜ—N(R^{e})₂, —(CR^{c}R^{d})ₜ—N(R^{e})—C(O)(R^{e}), —(CR^{c}R^{d})ₜ—C(O)N(R^{e})₂, and —(CR^{c}R^{d})ₜ—S(O)₂N(R^{e})₂; two adjacent substituents as represented by T, together with the carbon or nitrogen atom to which they are attached, optionally form a monocyclic ring selected from the group consisting of phenyl, heteroaryl, cycloalkyl and cycloalkenyl, and each of the monocyclic ring is independently further unsubstituted or substituted with 1, 2, 3, 4 or 5 substituents independently selected from the group consisting of alkyl, alkenyl, alkynyl, nitro, —CN, halogen, ethylenedioxy, methylenedioxy, haloalkyl, —OR^{e}, —O—C(O)(R^{e}), —S(R^{e}), —S(O)(R)^{f}, —S(O)₂(R^{f}), —C(O)(R^{e}), —C(O)O(R^{e}), —N(R^{e})₂, —N(R^{e})—C(O)(R^{e}), —C(O)N(R^{e})₂, —S(O)₂N(R^{e})₂, —(CR^{c}R^{d})ₜ—OR^{e}, —(CR^{c}R^{d})ₜ—O—C(O)(R^{e}), —(CR^{c}R^{d})ₜ—S(R^{e}), —(CR^{c}R^{d})ₜ—S(O)(R^{f}), —(CR^{c}R^{d})ₜ—S(O)₂(R^{f}), —(CR^{c}R^{d})ₜ—C(O)(R^{e}), —(CR^{c}R^{d})ₜ—C(O)O(R^{e}), —(CR^{c}R^{d})ₜ—N(R^{e})₂, —(CR^{c}R^{d})ₜ—N(R^{e})—C(O)(R^{e}), —(CR^{c}R^{d})ₜ—C(O)N(R^{e})₂, and —(CR^{c}R^{d})ₜ—S(O)₂N(R^{e})₂;

Z is C(O), C(H)(OH), C(alkyl)(OH), O, N(R^{e}), S(O), S(O)₂, or CH₂;

Y is O, N(CN), S, or C(H)(NO₂);

W is O or S;

X represents a substituent group selected from the group consisting of —C(O)OR⁵, —C(O)N(R⁵)₂, —CN, —C(═NOR⁵)N(R⁵)₂, —C(R⁶R⁷)OH, —C(O)—N(R⁵)(OR⁵), and tetrazolyl; with the proviso that when Z is C(O) or C(H)(OH), A and D are phenyl, and X is located on the carbon atom that is adjacent to the carbon atom bearing Z, then X is —CN, —C(═NOR⁵)N(R⁵)₂, —C(R⁶R⁷)OH, —C(O)—N(R⁵)(OR⁵), or tetrazolyl; and with the further proviso that when Z is C(O), A is pyridinyl or pyrimidinyl, D is phenyl, and X is located on the carbon atom that is adjacent to the carbon atom bearing Z, then X is not —C(O)OH;

R⁵, at each occurrence, is independently hydrogen, alkyl, or haloalkyl;

R⁶ and R⁷ are independently hydrogen or alkyl, or R⁶ and R⁷ together with the carbon atom to which they are attached, form a three- to six-membered, monocyclic ring selected from the group consisting of cycloalkyl and cycloalkenyl;

R⁴, at each occurrence, is independently aryl, heteroaryl, cycloalkyl, cycloalkenyl, or heterocycle; wherein each R⁴ is independently unsubstituted or substituted with 1, 2, 3, 4 or 5 substituents independently selected from the group consisting of alkyl, alkenyl, alkynyl, nitro, —CN, halogen, ethylenedioxy, methylenedioxy, haloalkyl, —OR^{e}, —O—C(O)(R^{e}), —S(R^{e}), —S(O)(R^{f}), —S(O)₂(R^{f}), —C(O)(R^{e}), —C(O)O(R^{e}), —N(R^{e})₂, —N(R^{e})—C(O)(R^{e}), —C(O)N(R^{e})₂, —S(O)₂N(R^{e})₂, —(CR^{c}R^{d})ₜ—OR^{e}, —(CR^{c}R^{d})ₜ—O—C(O)(R^{e}), —(CR^{c}R^{d})ₜ—S(R^{e}), —(CR^{c}R^{d})ₜ—S(O)(R^{f}), —(CR^{c}R^{d})ₜ—S(O)₂(R^{f}), —(CR^{c}R^{d})ₜ—C(O)(R^{e}), —(CR^{c}R^{d})ₜ—C(O)O(R^{e}), —(CR^{c}R^{d})ₜ—N(R^{e})₂, —(CR^{c}R^{d})ₜ—N(R^{e})—C(O)(R^{e}), —(CR^{c}R^{d})ₜ—C(O)N(R^{e})₂, and —(CR^{c}R^{d})ₜ—S(O)₂N(R^{e})₂;

R^{a}, at each occurrence, is independently hydrogen, alkyl, haloalkyl, R⁴, or —(CR^{g}R^{h})ᵤ—R⁴;

R^{b}, at each occurrence, is independently alkyl, haloalkyl, R⁴, or —(CR^{g}R^{h})ᵤ—R⁴;

R^{c}, R^{d}, R^{g}, and R^{h}, at each occurrence, are each independently hydrogen, halogen, alkyl or haloalkyl; or

R^{g} and R^{h}, together with the carbon atom to which they are attached, form a monocyclic, three- to six-membered cycloalkyl ring;

R^{e}, at each occurrence, is independently hydrogen, alkyl or haloalkyl;

R^{f}, at each occurrence, is independently alkyl or haloalkyl; and

u and t, at each occurrence, are each independently 1, 2, 3, or 4.

Another aspect of the invention provides methods of heating various diseases or conditions in a subject, preferably a human, wherein the methods include administering to the subject in need thereof a therapeutically effective amount of a compound of the invention as disclosed herein, or a pharmaceutically acceptable salt thereof, or a pharmaceutical compositions including a compound of the invention, or a pharmaceutically acceptable salt thereof, and a pharmaceutically acceptable carrier. In another aspect, the invention provides methods of preventing or treating a disease or condition related to elevated lipid levels, such as plasma lipid levels, especially elevated triacylglycerides levels, in a subject, especially human, afflicted with such elevated levels, including administering to the subject a therapeutically or prophylactically effective amount of a compound, or a pharmaceutically acceptable salt thereof, or a composition as disclosed herein. The invention also relates to novel compounds having therapeutic ability to reduce lipid levels in a subject (for example, mammal), especially triacylglycerides levels. In another aspect, the invention provides pharmaceutical compositions including the compound of the invention as disclosed herein, or a pharmaceutically acceptable salt thereof, and a pharmaceutically acceptable carrier. In one embodiment, the present invention relates to methods of treating various conditions in a subject (for example, mammal) including the step of administering to the subject a pharmaceutical composition including an amount of the compound of the invention, or a pharmaceutically acceptable salt thereof, that is effective in treating the target condition, and a pharmaceutically acceptable carrier.

### DETAILED DESCRIPTION OF THE INVENTION

For a variable that occurs more than one time in any substituent or in the compound of the invention or any other formulae herein, its definition at each occurrence is independent of its definition at every other occurrence. Combinations of substituents are permissible only if such combinations result in stable compounds. Stable compounds are compounds, which can be isolated in a useful degree of purity from a reaction mixture.

As used in the specification and the appended claims, unless specified to the contrary, the following terms have the meaning indicated:

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkyl” as used herein, means a straight or branched chain, saturated hydrocarbon containing from 1 to 10 carbon atoms. The term “lower alkyl” or “C₁₋₆ alkyl” means a straight or branched chain hydrocarbon containing 1, 2, 3, 4, 5, or 6 carbon atoms. The term “C₁₋₃ alkyl” means a straight or branched chain hydrocarbon containing 1, 2, or 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3 -dimethylpentyl, n-heptyl, n-octyl, n-nonyl, and n-decyl.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl” as used herein, means phenyl or a bicyclic aryl. The bicyclic aryl is naphthyl, or a phenyl fused to a monocyclic cycloalkyl, or a phenyl fused to a monocyclic cycloalkenyl. The phenyl and the bicyclic aryl groups of the present invention are unsubstituted or substituted. The bicyclic aryl is attached to the parent molecular moiety through any carbon atom contained within the bicyclic aryl. Representative examples of the aryl groups include, but are not limited to, dihydroindenyl, indenyl, naphthyl, dihydronaphthalenyl, and 5,6,7,8-tetrahydronaphthalenyl.

The term “cyano” as used herein means a —CN group

The term “cycloalkyl” or “cycloalkane” as used herein, means a monocyclic or bicyclic cycloalkyl. The monocyclic cycloalkyl has three to eight carbon atoms, zero heteroatom and zero double bond. Examples of monocyclic ring systems include cyclopropyl, cyclobutyl, cyclopentyl, cyclohexyl, cycloheptyl, and cyclooctyl. The bicyclic cycloalkyl is a monocyclic cycloalkyl fused to a monocyclic cycloalkyl, or a monocyclic cycloalkyl in which two non-adjacent carbon atoms of the monocyclic cycloalkyl are linked by an alkylene bridge of one, two, or three carbon atoms. The monocyclic and bicyclic cycloalkyls can be attached to the parent molecular moiety through any substitutable atom contained within the monocyclic and bicyclic cycloalkyl groups. The monocyclic and bicyclic cycloalkyl groups of the present invention can be unsubstituted or substituted.

The term “cycloalkenyl” or “cycloalkene” as used herein, means a monocyclic or a bicyclic hydrocarbon ring system. The monocyclic cycloalkenyl has four-, five-, six-, seven- or eight carbon atoms and zero heteroatom. The four-membered ring systems have one double bond, the five-or six-membered ring systems have one or two double bonds, and the seven- or eight-membered ring systems have one, two or three double bonds. Representative examples of monocyclic cycloalkenyl groups include, but are not limited to, cyclobutenyl, cyclopentenyl, cyclohexenyl, cycloheptenyl and cyclooctenyl. The bicyclic cycloalkenyl is a monocyclic cycloalkenyl fused to a monocyclic cycloalkyl group, or a monocyclic cycloalkenyl fused to a monocyclic cycloalkenyl group. Representative examples of the bicyclic cycloalkenyl groups include, but are not limited to, 4,5,6,7-tetrahydro-3aH-indene, octahydronaphthalenyl and 1,6-dihydro-pentalene. The monocyclic and the bicyclic cycloalkenyls can be attached to the parent molecular moiety through any substitutable atom contained within the groups, and can be unsubstituted or substituted.

The term “ethylenedioxy” as used herein, means a —O—(CH₂)₂—O— group wherein the oxygen atoms of the ethylenedioxy group are attached to two adjacent carbon atoms of a phenyl or naphthyl moiety, forming a six membered ring with the phenyl or naphthyl moiety that it is attached to.

The term “halo” or “halogen” as used herein, means —Cl, —Br, —I or —F.

The term “haloalkyl” as used herein, means an alkyl group, as defined herein, in which one, two, three, four, five or six hydrogen atoms are replaced by halogen. Representative examples of haloalkyl include, but are not limited to, difluoromethyl, chloromethyl, 2-fluoroethyl, trifluoromethyl, pentafluoroethyl, and 2-chloro-3-fluoropentyl.

The term “heterocycle” or “heterocyclic” as used herein, means a monocyclic heterocycle, or a bicyclic heterocycle. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N and S. The five-membered ring contains zero or one double bond and one, two or three heteroatoms selected from the group consisting of O, N and S. The six-membered ring contains zero, one or two double bonds and one, two or three heteroatoms selected from the group consisting of O, N and S. The seven- or eight-membered ring contains zero, one, two, or three double bonds and one, two or three heteroatoms selected from the group consisting of O, N and S. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyriolinyl, pyrrolidinyl, tetrahydrofuranyl, tetrahydropyranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, a monocyclic heterocycle fused to a monocyclic heterocycle. Representative examples of bicyclic heterocycles include, but are not limited to, 1,3-benzodithiolyl, benzopyranyl, benzothiopyranyl, 2,3-dihydrobenzofuranyl, 2,3-dihydrobenzothenyl, 2,3-dihydro-1H-indolyl, 2,3-dihydroisoindol-2-yl, 2,3-dihydroisoindol-3-yl, 1,3-dioxo-1H-isoindolyl, 2-(trifluoromethyl)-5,6-dihydroimidazo-[1,2-a]pyrazin-7(8H)-yl, 1-acetyl-2,3 -dihydro-1H-indo-6-yl, 3-(trifluoromethyl)-5,6-dihydro[1,2,4]triazolo[4,3-a]pyrazin-7(8H)-yl, 1,2,3,4-tetrahydroisoquinolin-2-yl, and 1,2,3,4-tetrahydroquinolinyl. The monocyclic and bicyclic heterocycles are connected to the parent molecular moiety through any carbon atom or any nitrogen atom contained within the monocyclic and bicyclic heterocycles, and can be unsubstituted or substituted.

The term “heteroaryl” as used herein, means a monocyclic heteroaryl, or a bicyclic heteroaryl. The monocyclic heteroaryl is a five- or six-membered ring. The five-membered ring contains two double bonds, and at least one heteroatom selected from oxygen, sulfur and nitrogen. The six-membered ring contains three double bonds and one, two, three or four nitrogen atoms. Representative examples of monocyclic heteroaryl include, but are not limited to, furanyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazynyl. The bicyclic heteroaryl is exemplified by a monocyclic heteroaryl fused to a phenyl, or a monocyclic heteroaryl fused to a monocyclic cycloalkyl, or a monocyclic heteroaryl fused to a monocyclic cycloalkenyl, or a monocyclic heteroaryl fused to a monocyclic heteroaryl, or a monocyclic heteroaryl fused to a monocyclic heterocycle. Representative examples of bicyclic heteroaryl groups include, but not limited to, benzofutanyl, benzothienyl, benzoxazolyl, benzimidazolyl, benzoxadiazolyl, 6,7-dihydro-1,3-benzothiazolyl, imidazo[1,2-a]pyridinyl, indazolyl, indolyl, isoindolyl, isoquinolinyl, naphthyridinyl, pyridoimidazolyl, quinolinyl, thiazolo[5,4-b]pyridin-2-yl, thiazolo[5,4-d]pyrimidin-2-yl, and 5,6,7,8-tetrahydroquinolin-5-yl. The monocyclic and the bicyclic heteroaryls are connected to the parent molecular moiety through any carbon atom or any nitrogen atom contained within the monocyclic and bicyclic heteroaryls, and can be substituted or unsubstituted.

The term “heteroatom” as used herein, means a nitrogen, oxygen or sulfur atom.

The term “methylenedioxy” as used herein, means a —O—(CH₂)—O— group wherein the oxygen atoms of the methylenedioxy group are attached to two adjacent carbon atoms of the phenyl or naphtyl ring, forming a five membered ring with the phenyl or naphthyl moiety that it is attached to.

The term “nitro” as used herein, means an —NO₂ group.

The term “mammal” includes humans and animals, such as cats, dogs, swine, cattle, horses, and the like.

The term “pharmaceutically acceptable ester,” as used herein, refers to esters of compounds of the invention which hydrolyze in vivo and include those that break down readily in the human body to leave the parent compound or a salt thereof. Examples of pharmaceutically acceptable, non-toxic esters of the invention include C₁₋₆ alkyl esters and C₅₋₇ cycloalkyl esters, although C₁₋₄ alkyl esters are preferred. Esters of the compounds of the invention can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, alkyl triflate, for example with methyl iodide, benzyl iodide, cyclopentyl iodide. They also can be prepared by reaction of the compound with an acid such as hydrochloric acid and an alkylcarboxylic acid such as acetic acid, or with acid and an arylcarboxylic acid such as benzoic acid.

The term “pharmaceutically acceptable amide,” as used herein, refers to non-toxic amides of the invention derived from ammonia, primary C₁₋₆ alkyl amines and secondary C₁₋₆ dialkyl amines. In the case of secondary amines, the amine can also be in the form of a 5- or 6-membered heterocycle containing one nitrogen atom. Amides derived from ammonia, C₁₋₃ alkyl primary amides and C₁₋₂ dialkyl secondary amides are preferred. Amides of the compounds of formula (I), (Ia), (Ib), (Ic), (Id), (Ie), (If), or (Ig), can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable esters are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, piperidine. They also can be prepared by reaction of the compound with an acid such as sulfuric acid and an alkylcarboxylic acid such as acetic acid, or with acid and an arylcarboxylic acid such as benzoic acid under dehydrating conditions as with molecular sieves added. The composition can contain a compound of the invention in the form of a pharmaceutically acceptable prodrug.

The term “pharmaceutically acceptable prodrug” or “prodrug” as used herein, represents those prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of the invention, for example, by hydrolysis in blood. A thorough discussion is provided in T. Higulchi and V. Stella, Pro-drugs as Novel Delivery Systems, V. 14 of the A C S Symposium Series, and in Edward B Roche, ed., Bioreversible Carriers in Drug Design, American Pharmaceutical Association and Pergamon Press (1987).

The term “pharmaceutically acceptable carrier” as used herein, means a non-toxic, solid, semi-solid or liquid filled, diluent, encapsulating material, or formulation auxiliary of any type. Examples of therapeutically suitable excipients include sugars; cellulose and derivatives thereof; oils; glycols; solutions; buffering, coloring, releasing, coating, sweetening, flavoring, and perfuming agents; and the like. These therapeutic compositions can be administered parenterally, intracisternally, orally, rectally, intravenously, or intraperitoneally.

The term “treatment” or “treating” includes any process, action, application, therapy, or the like, wherein a subject, including human, is provided medical aid with the object of improving the subject's condition, directly or indirectly, or slowing the progression of a condition or disorder in the subject.

Compounds of the invention have the formula (I) as described above.

Particular values of variable groups in compounds of formula (I) are as follows Such values can be used where appropriate with any of the other values, definitions, claims or embodiments defined hereinbefore or hereinafter.

In compounds of formula (I), Q is —C(═Y)N(R²)(R^{2a}), —C(═W)(R^{b}), —R^{b}, —S(O)₂(R^{b}), or —C(O)O(R^{b}).

In one embodiment, Q is —C(═Y)N(R²)(R^{2a}) wherein R², R^{2a} and Y are as described in the summary. R^{2a} is hydrogen or lower alkyl, particularly, R^{2a} is hydrogen of methyl; more particularly, R^{2a} is hydrogen. R² is alkyl, aryl, heteroaryl, cycloalkyl, cycloalkenyl or heterocycle; wherein each of the aryl, heteroaryl, cycloalkyl, cycloalkenyl or heterocycle is independently unsubstituted or substituted with substituents as described in the summary of the invention. In one embodiment, R² is aryl, heteroaryl or cycloalkyl, for example, R² is phenyl, thienyl, pyridinyl, cyclopentyl, cyclohexyl, cycloheptyl, cyclooctyl, adamantyl or 1,2,3,4-tetrahydronaphthalen-1-yl, wherein each R² is independently further unsubstituted or substituted with substituents as described in the summary section. Particularly, R² is phenyl, cyclohexyl, pyridinyl or thienyl, each of which is independently unsubstituted or substituted with substituents as described in the summary section, preferably, unsubstituted or substituted with 1 or 2 substituents selected from the group consisting of C₁₋₆ alkyl such as methyl, ethyl, and the like, —OR^{a} wherein R^{a} s C₁₋₆ alkyl such as methyl, ethyl, and the like, halogen (for example, fluoro, chloro, and the like), and haloalkyl such as trifluoromethyl.

Examples of Y are O, N(CN), S or C(H)(NO₂). In one embodiment, Y is O or S. In another embodiment, Y is O.

In another embodiment, Q is —C(═W)(R^{b}) wherein W and R^{b} are as described in the summary sections W is O or S. In one embodiment, W is O. R^{b} is alkyl, haloalkyl, R⁴, of —(CR^{g}R^{h})ᵤ—R⁴ wherein R⁴, R^{g}, R^{h} and u are as described in the summary. In one embodiment, R^{b} is R⁴, or —(CR^{g}R^{h})ᵤ—R⁴ wherein R⁴ is aryl or heteoraryl, each of which is independently unsubstituted or substituted as described in the summary section. For example, R⁴ is phenyl, thienyl or pyridinyl, each of which is unsubstituted or substituted as described in the summary. Examples of the substituents on R⁴ include, but are not limited to, C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl. In one embodiment, u is 1, one of R^{g} and R^{h} is hydrogen, and the other is hydrogen, C₁₋₆ alkyl such as methyl, and the like, or halogen such as fluoro and the like. In another embodiment, u is 1, and R^{g} and R^{h}, together with the carbon atom to which they are attached, form a monocyclic, three- to six-membered cycloalkyl ring (for example, cyclopropyl).

In another embodiment, Q is —R^{b} wherein R^{b} is as described in the summary section.

In yet another embodiment, Q is —S(O)₂(R^{b}) wherein R^{b} is as described in the summary section.

In yet another embodiment, Q is —C(O)O(R^{b}) wherein R^{b} is as described in the summary section.

R¹ is hydrogen, or lower alkyl such as, but not limited to, methyl. In one embodiment, R¹ is hydrogen.

A and D are each a monocyclic ring selected from the group consisting of phenyl, heteroaryl, cycloalkyl, and cycloalkenyl. In one embodiment, A and D are each independently a monocyclic ring selected from the group consisting of phenyl, heteroaryl, or cycloalkyl (for example, cyclohexyl). In another embodiment, A and D are both phenyl. In yet another embodiment, A is phenyl and D is monocyclic heteroaryl. In yet another embodiment, A is monocyclic heteroaryl, and D is phenyl. In yet another embodiment, A is cycloalkyl and D is phenyl. In a further embodiment, A and D are both monocyclic heteroaryl. Each of the rings as represented by A and D is optionally further substituted with 1, 2, 3, 4, or 5 substituents as represented by T and T is as described in the summary section. Examples of monocyclic heteroaryl ring include, but are not limited to, pyridinyl, pyrimidinyl, pyrazinyl, thieny, furanyl, pyrrolyl, thiazolyl, isothiazolyl, oxazolyl, isoxazolyl, imidazolyl, and pyrazolyl. In one embodiment, the heteroaryl ring is pyridinyl, thienyl or thiazolyl. Examples of the optional substituents on each of the rings A and D include, but are not limited to, C₁₋₆ alkyl such as methyl, ethyl, and the like, halogen such as fluoro, chloro, and the like, and haloalkyl (for example, trifluoromethyl, difluoromethyl). In one embodiment, A and D are each independently unsubstituted or further substituted with 1 or 2 substituents T wherein T is halogen (for example, fluoro).

m is 1, 2, 3, 4 or 5. In one embodiment, m is 2, 3 or 4.

Z is C(O), C(H)(OH), C(alkyl)(OH), O, N(R^{e}), S(O), S(O)₂ or CH₂, wherein R^{e} is as described in the summary section. In one embodiment, Z is C(O) or C(H)(OH). In another embodiment, Z is C(O). In yet another embodiment, Z is CH₂.

X represents a substituent group selected from the group consisting of —C(O)OR⁵, —C(O)N(R⁵)₂, —CN, —C(═NOR⁵)N(R⁵)₂, —C(R⁶R⁷)OH, —C(O)—N(R⁵)(OR⁵) and tetrazolyl wherein R⁵, R⁶ and R⁷ are as described in the summary of the invention, with the proviso that when Z is C(O) or C(H)(OH), A and D are phenyl, and X is located on the carbon atom that is adjacent to the carbon atom bearing Z, then X is —CN, —C(═NOR⁵)N(R⁵)₂, —C(R⁶R⁷)OH, —C(O)—N(R⁵)(OR⁵), or tetrazolyl; and with the further proviso that when Z is C(O), A is pyridinyl or pyrimidinyl, D is phenyl, and X is located on the carbon atom that is adjacent to the carbon atom bearing Z, then X is not —C(O)OH. In one embodiment, X is —C(O)OR⁵ —C((O)N(R⁵)₂, —CN, or —C(R⁶R⁷)OH wherein R⁵, R⁶ and R⁷ are each independently hydrogen of C₁₋₆ alkyl (for example, methyl), with the proviso that when X is located on the carbon atom that is adjacent to the carbon atom bearing Z, Z is C(O) or C(H)(OH), A and D are phenyl, then X is —CN or —C(R⁶R⁷)OH, and with the further proviso that when Z is C(O), A is pyridinyl or pyrimidinyl, D is phenyl, and X is located on the carbon atom that is adjacent to the carbon atom bearing Z, then X is not —C(O)OH. In another embodiment, X is —C(O)OH, with the proviso that when X is located on the carbon atom that is adjacent to the carbon atom bearing Z, and Z is C(O) or C(H)(OH), then A and D are not phenyl, and with the further proviso that when X is located on the carbon atom that is adjacent to the carbon atom bearing Z, Z is C(O), and A is pyridinyl or pyrimidinyl, then D is not phenyl.

n is 0, 1 or 2. In one embodiment, n is 0.

It is appreciated that the present invention contemplates compounds of formula (I) with combinations of the above embodiments, including particular, more particular and preferred embodiments

Accordingly, one aspect of the invention relates to a compound of formula (I), or a pharmaceutically acceptable salt, prodrug, salt of a prodrug, or a combination thereof, wherein Z is C(O) or C(H)(OH), X is —C(O)OR⁵, —C(O)N(R⁵)₂, —CN or —C(R⁶R⁷)OH, with the proviso that when X is located on the carbon atom adjacent to the carbon atom bearing Z, A and D are phenyl, then X is —CN or —C(R⁶R⁷)OH; and with the further proviso that when X is located on the carbon atom adjacent to the carbon atom bearing Z, Z is C(O), A is pyridinyl or pyrimidinyl, and D is phenyl, then X is not —C(O)OH; and R¹, R³, R⁵, R⁶, R⁷, Q, A, D, m, and n are as described in the summary section. Examples of R¹ are hydrogen and C₁₋₆ alkyl such as methyl. In one embodiment, R¹ is hydrogen. In one embodiment, A and D are each independently a monocyclic ring selected from the group consisting of phenyl, heteroaryl (for example, thienyl, pyridinyl, and thiazolyl), or cycloalkyl (for example, cyclohexyl). In another embodiment, A and I) are both phenyl. In yet another embodiment, A is phenyl and D is monocyclic heteroaryl (for example, thienyl, pyridinyl, and thiazolyl). In yet another embodiment, A is monocyclic heteroaryl (for example, thienyl, pyridinyl, and thiazolyl), and D is phenyl. In a further embodiment, A and D are both monocyclic heteroaryl (for example, thienyl, pyridinyl, and thiazolyl). Each of the rings as represented by A and D is optionally further substituted with 1, 2, 3, 4, or 5 substituents as represented by T and T is as described in the summary section.

Another aspect of the invention relates to a compound of formula (I), or a pharmaceutically acceptable salt, prodrug, salt of a prodrug, or a combination thereof, wherein Z is C(O) and X is —C(O)OR⁵ or —C(O)N(R⁵)₂, with the proviso that when X is located on the carbon atom adjacent to the carbon atom bearing Z, then A and D are not both phenyl, and with the proviso that when A is pyridinyl or pyrimidinyl, and D is phenyl, then X is not —C(O)OH, and R¹, R³, R⁵, Q, A, D, m, and n are as described in the summary of the invention. Examples of R⁵ include hydrogen and C₁₋₆ alkyl such as methyl, and ethyl. In one embodiment, X is —C(O)OH.

Of this group of compounds, examples of a subgroup include those wherein A is phenyl, and D is monocyclic heteroaryl, wherein each of A and D is optionally further substituted with 1, 2, 3, 4, or 5 substituents as represented by T, and T, Q, R¹, R³, m, and n are as described in the summary section. Examples of D as a monocyclic heteroaryl ring include, but are not limited to, pyridinyl, pyrimidinyl, pyrazinyl, thieny, furanyl, pyrrolyl, thiazolyl, isothiazolyl, oxazolyl, isoxazolyl, imidazolyl, and pyrazolyl, each of which is optionally further substituted as described herein. In one embodiment, D is pyridinyl, thienyl or thiazolyl, each of which is optionally further substituted with 1, 2, 3, 4, or 5 substituents as represented by T and T is as disclosed in the summary. Examples of T include, but are not limited to, C₁₋₆ alkyl such as methyl, ethyl, and the like, halogen such as fluoro, chloro, and the like, and haloalkyl (for example, trifluoromethyl or difluoromethyl) In one embodiment, A and D are each independently unsubstituted or further substituted with 1 or 2 substituents T wherein T is halogen (for example, fluoro). Examples of R¹ are hydrogen and C₁₋₆ alkyl such as methyl. In one embodiment, R¹ is hydrogen. In one embodiment, T¹ is 0. Q is —C(═Y)N(R²)(R ^{2a}), —C(═W)(R^{b}), —R^{b}, —S(O)₂(R^{b}), or —C(O)O(R^{b}) wherein Y, W, R², R^{2a}, and R^{b} are as described in the summary. In one embodiment, Q is —C(═Y)N(R²)(R^{2a}) wherein Y, R², and R^{2a} are as disclosed in the summary. Examples of Y include O, N(CN), S or C(H)NO₂. In one embodiment, Y is O or S. In another embodiment, Y is O. In one embodiment, R^{2a} is hydrogen or methyl. In another embodiment, R^{2a} is hydrogen. Examples of R² include phenyl, heteroaryl (for example, pyridinyl, pyrimidinyl, pyrazinyl, thieny, furanyl, pyrrolyl, thiazolyl, isothiazolyl, oxazolyl, isoxazolyl, imidazolyl, or pyrazolyl) and cycloalkyl (for example, cyclopentyl, cyclohexyl, cycloheptyl, cyclooctyl, adamantyl or 1,2,3,4-tetrahydronaphthalen-1-yl), each of which is independently optionally substituted as described in the summary. In one embodiment, R² is phenyl, thienyl, pyridinyl, of cyclohexyl, each of which is independently optionally further substituted as described in the summary. Examples of the optional substituents of R² include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl. In another embodiment, Q is —C(═W)(R^{b}) wherein W and R^{b} are as disclosed in the summary. In one embodiment, W is O. Examples of R^{b} include R⁴ and —(CR^{g}R^{h})ᵤ—R⁴ wherein R⁴, R^{g}, R^{h} and u are as defined in the summary. Examples of R⁴ include aryl (for example, phenyl) and heteroaryl, each of which is optionally further substituted as described in the summary. In one embodiment, R⁴ is phenyl, thienyl, or pyridinyl, each of which is optionally further substituted as described in the summary. Examples of the optional substituents of R⁴ include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl. In one embodiment, u is 1, one of R^{g} and R^{h} is hydrogen, and the other is hydrogen, C₁₋₆ alkyl such as methyl, and the like, or halogen (for example, fluoro). In another embodiment, u is 1, and R^{g} and R^{h}, together with the carbon atom to which they are attached, form a monocyclic, three- to six-membered cycloalkyl ring (for example, cyclopropyl).

Other examples of a subgroup include those wherein both A and D are monocyclic heteroaryl, wherein each A and D) is independently optionally further substituted with 1, 2, 3, 4, or 5 substituents as represented by T, and T, Q, R¹, R³, m, and n are as described in the summary section. Examples of the monocyclic heteroaryl ring include, but are not limited to, pyridinyl, pyrimidinyl, pyrazinyl, thieny, furanyl, pyrrolyl, thiazolyl, isothiazolyl, oxazolyl, isoxazolyl, imidazolyl, and pyrazolyl, each of which is optionally further substituted as described herein. In one embodiment, A and D are each independently pyridinyl, thienyl or thiazolyl, each of which is optionally further substituted with 1, 2, 3, 4, or 5 substituents as represented by T, and T is as disclosed in the summary. Examples of T include, but are not limited to, C₁₋₆ alkyl such as methyl, ethyl, and the like, halogen such as fluoro, chloro, and the like, and haloalkyl (for example, trifluoromethyl or difluoromethyl). In one embodiment, A and D are each independently unsubstituted or further substituted with 1 or 2 substituents T, wherein T is halogen (for example, fluoro). Examples of R¹ are hydrogen and C₁₋₆ alkyl such as methyl. In one embodiment, R¹ is hydrogen. In one embodiment, n is 0. Q is —C(═Y)N(R²)(R^{2a}), —C(═W)(R^{b}), —R^{b}, —S(O)₂(R^{b}), or —C(O)O(R^{b}) wherein Y, W, R², R^{2a}, and R^{b} are as described in the summary. In one embodiment, Q is —C(═Y)N(R²)(R^{2a}) wherein Y, R², and R^{2a} are as disclosed in the summary. Examples of Y include O, N(CN), S or C(H)NO₂. In one embodiment, Y is O or S. In another embodiment, Y is O. In one embodiment, R^{2a} is hydrogen or methyl. In another embodiment, R^{2a} is hydrogen. Examples of R² include phenyl, heteroaryl (for example, pyridinyl, pyrimidinyl, pyrazinyl, thieny, furanyl, pyrrolyl, thiazolyl, isothiazolyl, oxazolyl, isoxazolyl, imidazolyl, or pyrazolyl) and cycloalkyl (for example, cyclopentyl, cyclohexyl, cycloheptyl, cyclooctyl, adamantyl or 1,2,3,4-tetrahydronaphthalen-1-yl), each of which is independently optionally further substituted as described in the summary. In one embodiment, R² is phenyl, thienyl, pyridinyl, or cyclohexyl, each of which is independently optionally further substituted as described in the summary. Examples of the optional substituents of R² include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl. In another embodiment, Q is —C(═W)(R^{b}) wherein W and R^{b} are as disclosed in the summary. In one embodiment, W is O. Examples of R^{b} include R⁴ and —(CR^{g}R^{h})ᵤ—R⁴ wherein R⁴, R^{g}, R^{h} and u are as defined in the summary. Examples of R⁴ include aryl (for example, phenyl) and heteroaryl, each of which is optionally further substituted as described in the summary. In one embodiment, R⁴ is phenyl, thienyl, or pyridinyl, each of which is optionally further substituted as described in the summary. Examples of the optional substituents of R⁴ include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl. In one embodiment, u is 1, one of R^{g} and R^{h} is hydrogen, and the other is hydrogen, C₁₋₆ alkyl such as methyl, and the like, or halogen (for example, fluoro). In another embodiment, u is 1, and R^{g} and R^{h}, together with the carbon atom to which they are attached, form a monocyclic, three- to six-membered cycloalkyl ring (for example, cyclopropyl).

Yet other examples of a subgroup include those wherein A is cycloalkyl (for example, cyclohexyl) and D is phenyl, wherein each A and D is independently optionally further substituted with 1, 2, 3, 4, or 5 substituents as represented by T, and T, Q, R¹, R³, m, and n are as described in the summary section. In one embodiment, A is cyclohexyl and D is phenyl, each of which is optionally further substituted with 1, 2, 3, 4, or 5 substituents as represented by T, and T is as disclosed in the summary. Examples of T include, but are not limited to, C₁₋₆ alkyl such as methyl, ethyl, and the like, halogen such as fluoro, chloro, and the like, and haloalkyl (for example, trifluoromethyl or difluoromethyl). In one embodiment, A and D are each independently unsubstituted or further substituted with 1 or 2 substituents T, wherein T is halogen (for example, fluoro). Examples of R¹ are hydrogen and C₁₋₆ alkyl such as methyl. In one embodiment, R¹ is hydrogen. In one embodiment, n is 0. Q is —C(═Y)N(R²)(R^{2a}), —C(═W)(R^{b}), —R^{b}, —S(O)₂(R^{b}), or —C(O)O(R^{b}) wherein Y, W, R², R^{2a}, and R^{b} are as described in the summary. In one embodiment, Q is —C(═Y)N(R²)(R^{2a}) wherein Y, R², and R^{2a} are as disclosed in the summary. Examples of Y include O, N(CN), S or C(H)NO₂. In one embodiment, Y is O or S. In another embodiment, Y is O. In one embodiment, R^{2a} is hydrogen or methyl. In another embodiment, R^{2a} is hydrogen. Examples of R² include phenyl, heteroaryl (for example, pyridinyl, pyrimidinyl, pyrazinyl, thieny, furanyl, pyrrolyl, thiazolyl, isothiazolyl, oxazolyl, isoxazolyl, imidazolyl, or pyrazolyl) and cycloalkyl (for example, cyclopentyl, cyclohexyl, cycloheptyl, cyclooctyl, adamantyl or 1,2,3,4-tetrahydronaphthalen-1-yl), each of which is independently optionally further substituted as described in the summary. In one embodiment, R² is phenyl, thienyl, pyridinyl, or cyclohexyl, each of which is independently optionally further substituted as described in the summary. Examples of the optional substituents of R² include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl. In another embodiment, Q is —C(═W)(R^{b}) wherein W and R^{b} are as disclosed in the summary. In one embodiment, W is O. Examples of R^{b} include R⁴ and —(CR^{g}R^{h})ᵤ—R⁴ wherein R⁴, R^{g}, R^{h} and u are as defined in the summary. Examples of R⁴ include aryl (for example, phenyl) and heteroaryl, each of which is optionally further substituted as described in the summary. In one embodiment, R⁴ is phenyl, thienyl, or pyridinyl, each of which is optionally further substituted as described in the summary. Examples of the optional substituents of R⁴ include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl. In one embodiment, u is 1, one of R^{g} and R^{h} is hydrogen, and the other is hydrogen, C₁₋₆ alkyl such as methyl, and the like, or halogen (for example, fluoro). In another embodiment, u is 1, and R^{g} and R^{h}, together with the carbon atom to which they are attached, form a monocyclic, three- to six-membered cycloalkyl ring (for example, cyclopropyl).

Yet another aspect of the invention is related to a group of compounds having formula (I), or a pharmaceutically acceptable salt, prodrug, salt of a prodrug, or a combination thereof, wherein Z is C(O) and X is —C(R⁶R⁷)OH, and R¹, R³, R⁶, R⁷, Q, A, D, m, and n are as described in the summary of the invention. Examples of R⁶ and R⁷ include, but are not limited to, hydrogen and methyl. In one embodiment, X is —CH₂OH. In another embodiment, X is —C(CH₃)₂OH.

Of this group of compounds, examples of a subgroup include those wherein A and D are phenyl wherein each of the phenyl rings as represented by A and D is independently further unsubstituted or substituted with 1, 2, 3, 4, or 5 substituents as represented by T, and T, R¹, R³, Q, m, and n are as described in the summary. Examples of R¹ are hydrogen and C₁₋₆, alkyl such as methyl. In one embodiment, R¹ is hydrogen. In one embodiment, n is 0. In one embodiment, A and D are each independently unsubstituted or further substituted with 1 or 2 substituents T wherein T is halogen (for example, fluoro).

Of this group of compounds, examples of another subgroup include those wherein A is phenyl and D is monocyclic heteroaryl wherein each of the rings as represented by A and D is independently further unsubstituted or substituted with 1, 2, 3, 4, or 5 substituents as represented by T, and T, R¹, R³, Q, m, and n are as described in the summary. Examples of R¹ are hydrogen and C₁₋₆ alkyl such as methyl. In one embodiment, R¹ is hydrogen. In one embodiment, n is 0. Examples of D as a monocyclic heteroaryl ring are as disclosed hereinabove. In one embodiment, D is pyridinyl, thienyl or thiazolyl, each of which is optionally further substituted as disclosed hereinabove. In one embodiment, A and D are each independently unsubstituted or further substituted with 1 or 2 substituents T wherein T is halogen (for example, fluoro).

Of this group of compounds, examples of yet another subgroup include those wherein A is monocyclic heteroaryl and D is phenyl wherein each of the rings as represented by A and D is independently further unsubstituted or substituted with 1, 2, 3, 4, or 5 substituents as represented by T, and T, R¹, R³, Q, m, and n are as described in the summary. Examples of R¹ are hydrogen and C₁₋₆ alkyl such as methyl. In one embodiment, R¹ is hydrogen. In one embodiment, n is 0. Examples of A as a monocyclic heteroaryl ring are as disclosed hereinabove. In one embodiment, A is pyridinyl, thienyl or thiazolyl, each of which is optionally further substituted as disclosed hereinabove. In one embodiment, A and D are each independently unsubstituted or further substituted with 1 or 2 substituents T wherein T is halogen (for example, fluoro).

Of this group of compounds, examples of yet another subgroup include those wherein A and D are each a monocyclic heteroaryl wherein each of the rings as represented by A and D is independently further unsubstituted or substituted with 1, 2, 3, 4, or 5 substituents as represented by T, and T, R¹, R³, Q, m, and n are as described in the summary. Examples of R¹ are hydrogen and C₁₋₆ alkyl such as methyl. In one embodiment, R¹ is hydrogen. In one embodiment, n is 0. Examples of A and D as a monocyclic heteroaryl ring are as disclosed hereinabove. In one embodiment, A and D are each independently pyridinyl, thienyl or thiazolyl, each of which is optionally further substituted as disclosed hereinabove. In one embodiment, A and D are each independently unsubstituted or further substituted with 1 or 2 substituents T wherein T is halogen (for example, fluoro).

Of this group of compounds, examples of yet another subgroup include those wherein A is cycloalkyl (for example, cyclohexyl) and D is phenyl, each of which is independently unsubstituted or substituted as described in the preceding paragraph. Particular values of T, R¹, R³, Q, m, and n are as described in the preceding paragraph.

Yet another aspect of the invention is related to a group of compounds having formula (I), or a pharmaceutically acceptable salt, prodrug, salt of a prodrug, or a combination thereof, wherein Z is C(H)(OH) and X is —C(R⁶R⁷)OH, and R¹, R³, R⁶, R⁷, Q, A, D, m, and n are as described in the summary of the invention. Examples of R⁶ and R⁷ include, but are not limited to, hydrogen and methyl. In one embodiment, X is —CH₂OH. In another embodiment, X is —C(CH₃)₂OH.

Of this group of compounds, examples of a subgroup include those wherein A and D are phenyl wherein each of the phenyl rings as represented by A and D is independently further unsubstituted or substituted with 1, 2, 3, 4, or 5 substituents as represented by T, and T, R¹, R³, Q, m, and n are as described in the summary. Examples of R¹ are hydrogen and C₁₋₆ alkyl such as methyl. In one embodiment, R¹ is hydrogen. In one embodiment, n is 0. In one embodiment, A and D are each independently unsubstituted or substituted with 1 or 2 substituents T wherein T is halogen (for example, fluoro).

Of this group of compounds, examples of another subgroup include those wherein A is phenyl and D is monocyclic heteroaryl wherein each of the rings as represented by A and D is independently further unsubstituted or substituted with 1, 2, 3, 4, or 5 substituents as represented by T, and T, R¹, R³, Q, m, and n are as described in the summary. Examples of R¹ are hydrogen and C₁₋₆ alkyl such as methyl. In one embodiment, R¹ is hydrogen. In one embodiment, n is 0. Examples of D as a monocyclic heteroaryl ring are as disclosed hereinabove. In one embodiment, D is pyridinyl, thienyl or thiazolyl, each of which is optionally further substituted as disclosed hereinabove. In one embodiment, A and D are each independently unsubstituted or further substituted with 1 or 2 substituents T wherein T is halogen (for example, fluoro).

Of this group of compounds, examples of yet another subgroup include those wherein A is monocyclic heteroaryl and D is phenyl wherein each of the rings as represented by A and D is independently further unsubstituted or substituted with 1, 2, 3, 4, or 5 substituents as represented by T, and T, R¹, R³, Q, m, and n are as described in the summary. Examples of R¹ are hydrogen and C₁₋₄ alkyl such as methyl. In one embodiment, R¹ is hydrogen. In one embodiment, n is 0. Examples of A as a monocyclic heteroaryl ring are as disclosed hereinabove. In one embodiment, A is pyridinyl, thienyl or thiazolyl, each of which is optionally further substituted as disclosed hereinabove. In one embodiment, A and D are each independently unsubstituted or further substituted with 1 or 2 substituents T wherein T is halogen (for example, fluoro).

Of this group of compounds, examples of yet another subgroup include those wherein A and D are each a monocyclic heteroaryl wherein each of the rings as represented by A and D is independently further unsubstituted or substituted with 1, 2, 3, 4, or 5 substituents as represented by T, and T, R¹, R³, Q, m, and n are as described in the summary. Examples of R¹ are hydrogen and C₁₋₆ alkyl such as methyl. In one embodiment, R¹ is hydrogen. In one embodiment, n is 0. Examples of A and D as a monocyclic heteroaryl ring are as disclosed hereinabove. In one embodiment, A and D are each independently pyridinyl, thienyl or thiazolyl, each of which is optionally further substituted as disclosed hereinabove. In one embodiment, A and D are each independently unsubstituted or further substituted with 1 or 2 substituents T wherein T is halogen (for example, fluoro).

Of this group of compounds, examples of yet another subgroup include those wherein A is cycloalkyl (for example, cyclohexyl) and D is phenyl, each of which is independently unsubstituted or substituted as described in the preceding paragraph. Particular values of T, R¹, R³, Q, m, and n are as described in the preceding paragraph.

Yet a further aspect of the invention is related to a group of compounds having formula (I), or a pharmaceutically acceptable salt, prodrug, salt of a prodrug, or a combination thereof, wherein Z is CH₂ and X is —C(R⁶R⁷)OH, and R¹, R³, R⁶, R⁷, Q, A, D, m, and n are as described in the summary of the invention. Examples of R⁶ and R⁷ include, but are not limited to, hydrogen and methyl. In one embodiment, X is —CH₂OH. In another embodiment, X is —C(CH₃)₂OH.

Of this group of compounds, examples of a subgroup include those wherein A and D are phenyl wherein each of the phenyl rings as represented by A and D is independently further unsubstituted or substituted with 1, 2, 3, 4, or 5 substituents as represented by T, and T, R¹, R³, Q, m, and n are as described in the summary. Examples of R¹ are hydrogen and C₁₋₆ alkyl such as methyl. In one embodiment, R¹ is hydrogen. In one embodiment, n is 0. In one embodiment, A and D are each independently unsubstituted or substituted with 1 or 2 substituents T wherein T is halogen (for example, fluoro). Q is —C(═Y)N(R²)(R^{2a}), —C(═W)(R^{b}), —R^{b}, —S(O)₂(R^{b}), or —C(O)O(R^{b}) wherein Y, W, R ², R^{2a}, and R^{b} are as described in the summary. In one embodiment, Q is —C(═Y)N(R²)(R^{2a}) wherein Y, R², and R^{2a} are as disclosed in the summary. Examples of Y include O, N(CN), S or C(H)NO₂. In one embodiment, Y is O or S. In another embodiment, Y is O. In one embodiment, R^{2a} is hydrogen or methyl. In another embodiment, R², is hydrogen. Examples of R² include phenyl, heteroaryl (for example, pyridinyl, pyrimidinyl, pyrazinyl, thieny, furanyl, pyrrolyl, thiazolyl, isothiazolyl, oxazolyl, isoxazolyl, imidazolyl, or pyrazolyl) and cycloalkyl (for example, cyclopentyl, cyclohexyl, cycloheptyl, cyclooctyl, adamantyl or 1,2,3,4-tetrahydronaphthalen-1-yl), each of which is independently optionally substituted as described in the summary. In one embodiment, R² is phenyl, thienyl, pyridinyl, or cyclohexyl, each of which is independently optionally substituted as described in the summary. Examples of the optional substituents of R² include C₁₋₆ alkyl such as methyl, ethyl, and the like, —OR wherein R^{a} is C₁₋₆ alkyl such as methyl, ethyl, and the like, halogen (for example, fluoro, chloro, and the like), and haloalkyl such as trifluoromethyl.

Yet a further aspect of the invention is related to a group of compounds having formula (I), or a pharmaceutically acceptable salt, prodrug, salt of a prodrug, or a combination thereof, wherein Z is CO, X is —CN, and R¹, R³, Q, A, D, m, and n are as defined in the summary.

Of this group of compounds, examples of a subgroup include those wherein A and D are phenyl wherein each of the phenyl rings as represented by A and D is independently further unsubstituted or substituted with 1, 2, 3, 4, or 5 substituents as represented by T, and T, R¹, R³, Q, m, and n are as described in the summary. Examples of R¹ are hydrogen and C₁₋₆ alkyl such as methyl. In one embodiment, R¹ is hydrogen. In one embodiment, n is 0. In one embodiment, A and D are each independently unsubstituted or further substituted with 1 or 2 substituents T wherein T is halogen (for example, fluoro). Q is —C(═Y)N(R²)(R^{2a}), —C(═W)(R^{b}), —R^{b}, —S(O)₂(R^{b}), or —C(O)O(R^{b}) wherein Y, W, R², R^{2a}, and R^{b} are as described in the summary. In one embodiment, Q is —C(═Y)N(R²)(R^{2a}) wherein Y, R², and R^{2a} are as disclosed in the summary. Examples of Y include O, N(CN), S or C(H)NO₂. In one embodiment, Y is O or S. In another embodiment, Y is O. In one embodiment, R^{2a} is hydrogen or methyl. In another embodiment, R^{2a} is hydrogen. Examples of R² include phenyl, heteroaryl (for example, pyridinyl, pyrimidinyl, pyrazinyl, thieny, furanyl, pyrrolyl, thiazolyl, isothiazolyl, oxazolyl, isoxazolyl, imidazolyl, or pyrazolyl) and cycloalkyl (for example, cyclopentyl, cyclohexyl, cycloheptyl, cyclooctyl, adamantyl or 1,2,3,4-tetrahydronaphthalen-1-yl), each of which is independently optionally substituted as described in the summary. In one embodiment, R² is phenyl, thienyl, pyridinyl, or cyclohexyl, each of which is independently optionally substituted as described in the summary. Examples of the optional substituents of R² include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl.

Another aspect of the invention provides a group of compounds having formula (Ia), or a pharmaceutically acceptable salt, prodrug, salt of a prodrug, or a combination thereof, 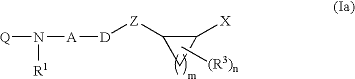
wherein R¹, R³, X, Q, Z, A, D, m, and n, are described as in formula (I) in the summary. It is appreciated that particular values of variable groups (for example, R¹, R³, X, Q, Z, A, D, m, and n) in compounds of formula (Ia), embodiments of such groups and combinations of embodiments, including particular; and more particular embodiments as described hereinabove in formula (I), are also contemplated for compounds of formula (Ia).

Thus, a further aspect of the invention is related to a group of compounds of formula (Ia), or a pharmaceutically acceptable salt, prodrug, salt of a prodrug, or a combination thereof, wherein Z is C(O) or C(H)(OH), X is —C(O)OR⁵, —C(O)N(R⁵)₂, —CN or —C(R⁶R⁷)OH, with the proviso that when A and D are phenyl, then X is CN or —C(R⁶R⁷)OH; and with the further proviso that when Z is C(O), A is pyridinyl or pyrimidinyl, and D is phenyl, then X is not —C(O)OH; and R¹, R³, R⁵, R⁶, R⁷, Q, A, D, m, and n are as described in the summary section. Examples of R¹ are hydrogen and C₁₋₆ alkyl such as methyl. In one embodiment, R¹ is hydrogens. In one embodiment, A and D are each independently a monocyclic ring selected from the group consisting of phenyl, heteroaryl (for example, thienyl, pyridinyl, and thiazolyl), or cycloalkyl (for example, cyclohexyl). In another embodiment, A and D are both phenyl. In yet another embodiment, A is phenyl and D is monocyclic heteroaryl (for example, thienyl, pyridinyl, and thiazolyl). In yet another embodiment, A is monocyclic heteroaryl (for example, thienyl, pyridinyl, and thiazolyl), and D is phenyl. In a further embodiment, A and D are both monocyclic heteroaryl (for example, thienyl, pyridinyl, and thiazolyl). Each of the rings as represented by A and D is optionally further substituted with 1, 2, 3, 4, or 5 substituents as represented by T and T is as described in the summary section.

Another aspect of the invention relates to a compound of formula (Ia), or a pharmaceutically acceptable salt, prodrug, salt of a prodrug, or a combination thereof, wherein Z is C(O) and X is —C(O)OR⁵ or —C(O)N(R⁵)₂, with the proviso that A and D are not both phenyl, and with the proviso that when A is pyridinyl or pyrimidinyl, and D is phenyl, then X is not C(O)OH, and R¹, R³, R⁵, Q, A, D, m, and n are as described in the summary of the invention. Examples of R⁵ include hydrogen and C₁₋₆ alkyl such as methyl, and ethyl. In one embodiment, X is —C(O)OH.

Of this group of compounds, examples of a subgroup include those wherein A is phenyl, and D is monocyclic heteroaryl, wherein each of A and D is optionally further substituted with 1, 2, 3, 4, or 5 substituents as represented by T, and T, Q, R³, m, and n are as described in the summary section. Examples of D as a monocyclic heteroaryl ring include, but are not limited to, pyridinyl, pyrimidinyl, pyrazinyl, thieny, furanyl, pyrrolyl, thiazolyl, isothiazolyl, oxazolyl, isoxazolyl, imidazolyl, and pyrazolyl, each of which is optionally further substituted as described herein. In one embodiment, D is pyridinyl, thienyl or thiazolyl, each of which is optionally further substituted with 1, 2, 3, 4, or 5 substituents as represented by T and T is as disclosed in the summary. Examples of T include, but are not limited to, C₁₋₆ alkyl such as methyl, ethyl, and the like, halogen such as fluoro, chloro, and the like, and haloalkyl (for example, trifluoromethyl or difluoromethyl). In one embodiment, A and D are each independently unsubstituted or further substituted with 1 or 2 substituents T wherein T is halogen (for example, fluoro). Examples of R^{t} are hydrogen and C₁₋₆ alkyl such as methyl. In one embodiment, R¹ is hydrogen. In one embodiment, n is 0. Q is —C(═Y)N(R²)(R^{2a}), —C(═W)(R^{b}), —R^{b}, S(O)₂(R^{b}), or —C(O)O(R^{b}) wherein Y, W, R², R^{2a}, and R^{b} are as described in the summary. In one embodiment, Q is —C(═Y)N(R²)(R^{2a}, wherein Y, R², and R² are as disclosed in the summary. Examples of Y include O, N(CN), S or C(H)NO₂. In one embodiment, Y is O or S. In another embodiment, Y is O. In one embodiment, R^{2a} is hydrogen or methyl. In another embodiment, R^{2a} s hydrogen. Examples of R² include phenyl, heteroaryl (for example, pyridinyl, pyrimidinyl, pyrazinyl, thieny, furanyl, pyrrolyl, thiazolyl, isothiazolyl, oxazolyl, isoxazolyl, imidazolyl, or pyrazolyl) and cycloalkyl (for example, cyclopentyl, cyclohexyl, cycloheptyl, cyclooctyl, adamantyl or 1,2,3,4-tetrahydronaphthalen-1-yl), each of which is independently optionally substituted as described in the summary. In one embodiment, R² is phenyl, thienyl, pyridinyl, or cyclohexyl, each of which is independently optionally substituted as described in the summary. Examples of the optional substituents of R² include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl. In another embodiment, Q is —C(═W)(R^{b}) wherein W and R^{b} are as disclosed in the summary. In one embodiment, W is O. Examples of R^{b} include R⁴ and —(CR^{g}R^{h})ᵤ—R⁴ wherein R⁴, R^{g}, R^{h} and u are as defined in the summary. Examples of R⁴ include aryl (for example, phenyl) and heteroaryl, each of which is optionally further substituted as described in the summary. In one embodiment, R⁴ is phenyl, thienyl, or pyridinyl, each of which is optionally further substituted as described in the summary. Examples of the optional substituents of R⁴ include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl. In one embodiment, u is 1, one of R^{g} and R^{h} is hydrogen, and the other is hydrogen, C₁₋₆ alkyl such as methyl, and the like, or, halogen (for example, fluoro). In another embodiment, u is 1, and R^{g} and R^{h}, together with the carbon atom to which they are attached, form a monocyclic, three- to six-membered cycloalkyl ring (for example, cyclopropyl).

Other examples of a subgroup include those wherein both A and D are monocyclic heteroaryl, wherein each A and D is independently optionally further substituted with 1, 2, 3, 4, or 5 substituents as represented by T, and T, Q, R³, m, and n are as described in the summary section. Examples of the monocyclic heteroaryl ring include, but are not limited to, pyridinyl, pyrimidinyl, pyrazinyl, thieny, furanyl, pyrrolyl, thiazolyl, isothiazolyl, oxazolyl, isoxazolyl, imidazolyl, and pyrazolyl, each of which is optionally further substituted as described herein. In one embodiment, A and D are each independently pyridinyl, thienyl or thiazolyl, each of which is optionally further substituted with 1, 2, 3, 4, or 5 substituents as represented by T and T is as disclosed in the summary. Examples of T include, but are not limited to, C₁₋₆ alkyl such as methyl, ethyl, and the like, halogen such as fluoro, chloro, and the like, and haloalkyl (for example, trifluoromethyl or difluoromethyl). In one embodiment, A and D are each independently unsubstituted or further substituted with 1 or 2 substituents T wherein T is halogen (for example, fluoro). Examples of R¹ are hydrogen and C₁₋₆ alkyl such as methyl. In one embodiment, R¹ is hydrogen. In one embodiment, n is 0. Q is —C(═Y)N(R²)(R^{2a}), —C(═W)(R^{b}), —R^{b}, —S(O)₂(R^{b}), or —C(O)O(R^{b}) wherein Y, W, R², R^{2a}, and R^{b} are as described in the summary. In one embodiment, Q is —C(═Y)N(R²)(R^{2a}) wherein Y, R², and R^{2a} are as disclosed in the summary. Examples of Y include O, N(CN), S or C(H)NO₂. In one embodiment, Y is O or S. In another embodiment, Y is O. In one embodiment, R², is hydrogen or methyl. In another embodiment, R^{2a} is hydrogen. Examples of R² include phenyl, heteroaryl (for example, pyridinyl, pyrimidinyl, pyrazinyl, thieny, furanyl, pyrrolyl, thiazolyl, isothiazolyl, oxazolyl, isoxazolyl, imidazolyl, or pyrazolyl) and cycloalkyl (for example, cyclopentyl, cyclohexyl, cycloheptyl, cyclooctyl, adamantyl or 1,2,3,4-tetrahydronaphthalen-1-yl), each of which is independently optionally substituted as described in the summary. In one embodiment, R² is phenyl, thienyl, pyridinyl, or cyclohexyl, each of which is independently optionally substituted as described ill the summary. Examples of the optional substituents of R² include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl In another embodiment, Q is —C(═W)(R^{b}) wherein W and R^{b} are as disclosed in the summary. In one embodiment, W is O. Examples of R^{b} include R and —(CR^{g}R^{h})ᵤ—R⁴ wherein R⁴, R^{g}, R^{h} and u are as defined in the summary. Examples of R⁴ include aryl (for example, phenyl) and heteroaryl, each of which is optionally further substituted as described in the summary. In one embodiment, R⁴ is phenyl, thienyl, or pyridinyl, each of which is optionally further substituted as described in the summary. Examples of the optional substituents of R⁴ include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl. In one embodiment, u is 1, one of R^{g} and R^{h} is hydrogen, and the other is hydrogen, C₁₋₆ alkyl such as methyl, and the like, or halogen (for example, fluoro). In another embodiment, u is 1, and R^{g} and R^{h}, together with the carbon atom to which they are attached, form a monocyclic, three- to six-membered cycloalkyl ring (for example, cyclopropyl).

Examples of yet another subgroup include those wherein A is cycloalkyl (for example, cyclohexyl) and D is phenyl, each of which is independently unsubstituted or substituted as described in the preceding paragraph. Particular values of T, R¹, R³, Q, m, and n are as described in the preceding paragraph.

A further aspect of the invention is related to a compound of formula (I), or a pharmaceutically acceptable salt, prodrug, salt of a prodrug, or a combination thereof, wherein Z is C(O) and m is 3, with the proviso that when A is phenyl, and D is phenyl, then X is —CN, —C(═NOR⁵)N(R⁵)₂, —C(R⁶R⁷)OH, —C(O)—N(R⁵)(OR⁵), or tetrazolyl, and with the further proviso that when A is pyridinyl or pyrimidinyl, and D is phenyl, then X is not —C(O)OH. Such compound can exist as the cis isomers or trans isomers. One embodiment is directed to the trans isomers as represented by formula (Ib). It is understood that the structural drawing of (Ib) encompasses not only one particular trans isomer as depicted in (Ib), but also other trans isomers (for example, (Ic)), and mixtures thereof (including racemates).

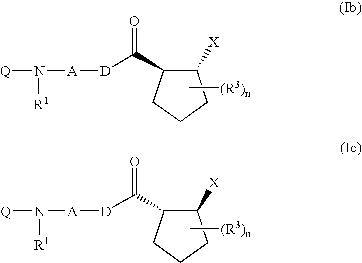

wherein Q, A, D, X, R¹, R³, and n in formula (Ib) and (Ic) are as described in formula (I). It is understood that particular values of the various groups (for example, Q, A, D, X, R¹, R³, and n), embodiments and combinations of embodiments of the groups, including particular, and more particular embodiments as described in formula (I) are also contemplated for compounds of formulae (Ib) and (Ic).

A further aspect of the invention is related to a compound of formula (I), or a pharmaceutically acceptable salt, prodrug, salt of a prodrug, or a combination thereof, wherein m is 2, and Z is C(O), with the proviso that when A is phenyl, and D is phenyl, then X is —CN, —C(═NOR⁵)N(R⁵)₂, —C(R⁶R⁷)OH, —C(O)—N(R⁵)(OR⁵), or tetrazolyl, and with the further proviso that when A is pyridinyl or pyrimidinyl, and D is phenyl, then X is not —C(O)OH. Such compound can exist as the cis isomers or trans isomers. One embodiment is directed to the trans isomers as represented by formula (Id). It is understood that the structural drawing of (Id) encompasses not only one particular trans isomer as depicted in (Id), but also other trans isomers (for example, (Ie)), and mixtures thereof (including racemates).

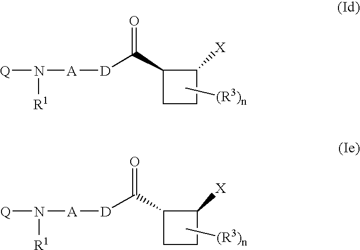

wherein Q, A, D, X, R¹, R³, and n in formula (Id) and (Ie) are as described in formula (I). It is understood that particular values of the various groups (for example, Q, A, D, X, R¹, R³, and n), embodiments and combinations of embodiments of the groups, including particular, and more particular embodiments as described in formula (I) are also contemplated for compounds of formulae (Id) and (Ie).

Thus, examples of compounds of formula (Ib) or (Id) include those wherein X is C(O)O(R⁵) or C(O)N(R⁵)₂ wherein R⁵, Q, A, D, R¹, R³, and n are as described in the summary, with the proviso that when A is phenyl, then D is not phenyl, and with the further proviso that when A is pyridinyl or pyrimidinyl, and D is phenyl, then and X is not —COOH Examples of R⁵ include hydrogen and C₁₋₆ alkyl such as methyl, and ethyl. In one embodiment, X is —C(O)OH.

Of this group of compounds, examples of a subgroup include those wherein A is phenyl, and D is a monocyclic heteroaryl, wherein each of A and D is optionally further substituted with 1, 2, 3, 4, or 5 substituents as represented by T, and T, Q, R¹, R³, and n are as described in the summary section. Examples of D as a monocyclic heteroaryl ring include, but are not limited to, pyridinyl, pyrimidinyl, pyrazinyl, thieny, furanyl, pyrrolyl, thiazolyl, isothiazolyl, oxazolyl, isoxazolyl, imidazolyl, and pyrazolyl, each of which is optionally further substituted as described herein. In one embodiment, D is pyridinyl, thienyl or thiazolyl, each of which is optionally further substituted with 1, 2, 3, 4, or 5 substituents as represented by T and T is as disclosed in the summary. Examples of T include, but are not limited to, C₁₋₆ alkyl such as methyl, ethyl, and the like, halogen such as fluoro, chloro, and the like, and haloalkyl (for example, trifluoromethyl or difluoromethyl). In one embodiment, A and D are each independently unsubstituted or further substituted with 1 or 2 substituents T wherein T is halogen (for example, fluoro). Examples of R¹ are hydrogen and C₁₋₆ alkyl such as methyl. In one embodiment, R¹ is hydrogen. In one embodiment, n is 0. Q is —C(═Y)N(R²)(R^{2a}), —C(═W)(R^{b}), —R^{b}, —S(O)₂(R^{b}), or —C(O)O(R^{b}) wherein Y, W, R², R^{2a}, and R^{b} are as described in the summary. In one embodiment, Q is —C(═Y)N(R²)(R^{2a}) wherein Y, R², and R^{2a} are as disclosed in the summary. Examples of Y include O, N(CN), S or C(H)NO₂. In one embodiment, Y is O or S. In another embodiment, Y is O. In one embodiment, R^{2a} is hydrogen or methyl. In another embodiment, R^{2a} is hydrogen. Examples of R² include phenyl, heteroaryl (for example, pyridinyl, pyrimidinyl, pyrazinyl, thieny, furanyl, pyrrolyl, thiazolyl, isothiazolyl, oxazolyl, isoxazolyl, imidazolyl, or pyrazolyl) and cycloalkyl (for example, cyclopentyl, cyclohexyl, cycloheptyl, cyclooctyl, adamantyl or 1,2,3,4-tetrahydronaphthalen-1-yl), each of which is independently optionally substituted as described in the summary. In one embodiment, R² is phenyl, thienyl, pyridinyl, or cyclohexyl, each of which is independently optionally substituted as described in the summary. Examples of the optional substituents of R² include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl. In another embodiment, Q is —C(═W)(R^{b}) wherein W and R^{b} are as disclosed in the summary. In one embodiment, W is O. Examples of R^{b} include R⁴ and —(CR^{g}R^{h})ᵤ—R⁴ wherein R⁴, R^{g}, R^{h} and u are as defined in the summary. Examples of R⁴ include aryl (for example, phenyl) and heteroaryl, each of which is optionally further substituted as described in the summary. In one embodiment, R⁴ is phenyl, thienyl, or pyridinyl, each of which is optionally further substituted as described in the summary. Examples of the optional substituents of R⁴ include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl. In one embodiment, u is 1, one of R^{g} and R^{h} is hydrogen, and the other is hydrogen, C₁₋₆ alkyl such as methyl, and the like, or halogen (for example, fluoro). In another embodiment, u is 1, and R^{g} and R^{h}, together with the carbon atom to which they are attached, form a monocyclic, three- to six-membered cycloalkyl ring (for example, cyclopropyl).

Other examples of a subgroup include those wherein both A and D are monocyclic heteroaryl, wherein each A and D is independently optionally further substituted with 1, 2, 3, 4, or 5 substituents as represented by T, and T, Q, R¹, R³, and n are as described in the summary section. Examples of the monocyclic heteroaryl ring include, but are not limited to, pyridinyl, pyrimidinyl, pyrazinyl, thieny, furanyl, pyrrolyl, thiazolyl, isothiazolyl, oxazolyl, isoxazolyl, imidazolyl, and pyrazolyl, each of which is optionally further substituted as described herein. In one embodiment, A and D are each independently pyridinyl, thienyl or thiazolyl, each of which is optionally further substituted with 1, 2, 3, 4, or 5 substituents as represented by T and T is as disclosed in the summary. Examples of T include, but are not limited to, C₁₋₆ alkyl such as methyl, ethyl, and the like, halogen such as fluoro, chloro, and the like, and haloalkyl (for example, trifluoromethyl or difluoromethyl). In one embodiment, A and D are each independently unsubstituted or further substituted with 1 or 2 substituents T wherein T is halogen (for example, fluoro). Examples of R¹ are hydrogen and C₁₋₆ alkyl such as methyl. In one embodiment, R¹ is hydrogen. In one embodiment, n is 0. Q is —C(═Y)N(R²)(R^{2a}), —C(═W)(R^{b}), —R^{b}, —S(O)₂(R^{b}), or —C(O)O(R^{b}) wherein Y, W, R², R^{2a}, and R^{b} are as described in the summary. In one embodiment, Q is —C(═Y)N(R²)(R^{2a}) wherein Y, R², and R^{2a} are as disclosed in the summary. Examples of Y include O, N(CN), S or C(H)NO₂. In one embodiment, Y is O or S. In another embodiment, Y is O. In one embodiment, R^{2a} is hydrogen or methyl. In another embodiment, R^{2a} is hydrogen. Examples of R² include phenyl, heteroaryl (for example, pyridinyl, pyrimidinyl, pyrazinyl, thieny, furanyl, pyrrolyl, thiazolyl, isothiazolyl, oxazolyl, isoxazolyl, imidazolyl, or pyrazolyl) and cycloalkyl (for example, cyclopentyl, cyclohexyl, cycloheptyl, cyclooctyl, adamantyl or 1,2,3,4-tetrahydronaphthalen-1-yl), each of which is independently optionally substituted as described in the summary. In one embodiment, R² is phenyl, thienyl, pyridinyl, or cyclohexyl, each of which is independently optionally substituted as described in the summary. Examples of the optional substituents of R² include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl. In another embodiment, Q is —C(W)(R^{b}) wherein W and R^{b} are as disclosed in the summary. In one embodiment, W is O. Examples of R^{b} include R⁴ and —(CR^{g}R^{h})ᵤ—R⁴ wherein R⁴, R^{g}, R^{h} and u are as defined in the summary. Examples of R⁴ include aryl (for example, phenyl) and heteroaryl, each of which is optionally further substituted as described in the summary. In one embodiment, R⁴ is phenyl, thienyl, or pyridinyl, each of which is optionally further substituted as described in the summary. Examples of the optional substituents of R⁴ include C₁₋₆ alkyl such as methyl, ethyl, and the like, —OR^{a} wherein R^{a} is C₁₋₆ alkyl such as methyl, ethyl, and the like, halogen (for example, fluoro, chloro, and the like), and haloalkyl such as trifluoromethyl. In one embodiment, u is 1, one of R^{g} and R^{h} is hydrogen, and the other is hydrogen, C₁₋₆ alkyl such as methyl, and the like, or halogen (for example, fluoro). In another embodiment, u is 1, and R^{g} and R^{h}, together with the carbon atom to which they are attached, form a monocyclic, three- to six-membered cycloalkyl ring (for example, cyclopropyl).

Yet other examples of a subgroup include those wherein A is cycloalkyl (for example, cyclohexyl) and D is phenyl, wherein each A and D is independently optionally further substituted with 1, 2, 3, 4, or 5 substituents as represented by T, and T, Q, R¹, R³, and n are as described in the summary section. Particular values of T, Q, R¹, R³, and n are as described in the preceding paragraph.

Exemplary compounds of the present invention include, but are not limited to the following:

N-(3-chlorophenyl)-N′-(4′-{(R)-hydroxy[(1R,2R)-2-(hydroxymethyl)cyclopentyl]methyl}-1,1′-biphenyl-4-yl)urea;

N-(3-chlorophenyl)-N′-(4′-{(S)-hydroxy[({(1R,2R)-2-(hydroxymethyl)cyclopentyl]methyl}-1,1′-biphenyl-4-yl)urea;

N-(3-chlorophenyl)-N′-(4′-{[(1R,2R)-2-(1-hydroxy-1-methylethyl)cyclopentyl]carbonyl}-1,1′-biphenyl-4-yl)urea;

N-(4′-{[(1R,2R)-2-(1-hydroxy-1-methylethyl)cyclopentyl]carbonyl}-1,1′-biphenyl-4-yl)-N′-phenylurea;

N-(4′-{(S)-hydroxy[(1R,2R)-2-(hydroxymethyl)cyclopentyl]methyl}-1,1′-biphenyl-4-yl)-N′-phenylurea;

N-(4′-{(R)-hydroxy[(1R,2R)-2-(hydroxymethyl)cyclopentyl]methyl}-1,1′-biphenyl-4-yl)-N′-phenylurea;

N-(4′-{[(1R,2R)-2-(hydroxymethyl)cyclopentyl]methyl}-1,1′-biphenyl-4-yl)-N′-phenylurea;

methyl(1R,2R)-2-{4-[5-{[(3-chlorophenyl)amino]carbonyl}amino)thien-2-yl]benzoyl}cyclopentanecarboxylate;

methyl(1R,2R)-2-(4-{5-[(anilinocarbonyl)amino]thien-2-yl}benzoyl)cyclopentanecarboxylate;

methyl(1R,2R)-2-(4-{5-[({[3-(trifluoromethyl)phenyl]amino}carbonyl)amino]thien-2-yl}benzoyl)cyclopentanecarboxylate;

(1R,2R)-2-{4-[5-({[(3-chlorophenyl)amino]carbonyl}amino)thien-2-yl]benzoyl}cyclopentanecarboxylic acid;

(1R,2R)-2-(4-{5-[(anilinocarbonyl)amino]thien-2-yl}benzoyl)cyclopentanecarboxylic acid;

(1R,2R)-2-(4-{5-[({[3-(trifluoromethyl)phenyl]amino}carbonyl)amino]thien-2-yl}benzoyl)cyclopentanecarboxylic acid;

N-(4′-{[(1R,2R)-2-cyanocyclopentyl]carbonyl}-1,1′-biphenyl-4-yl)-N′-phenylurea;

trans-2-{4-[4-({[(3-chlorophenyl)amino]carbonyl}amino)cyclohexyl]benzoyl}cyclopentanecarboxylic acid;

methyl(1R,2R)-2-(4-{6-[(anilinocarbonyl)amino]pyridin-3-yl}benzoyl)cyclopentanecarboxylate;

Trans-2-[(5-{4-[(anilinocarbonyl)amino]phenyl}pyridin-2-yl)carbonyl]cyclopentanecarboxylic acid;

Trans-2-[(5-{4-[({[3-(trifluoromethyl)phenyl]amino}carbonyl)amino]phenyl}pyridin-2-yl)carbonyl]cyclopentanecarboxylic acid;

Trans-2-({5-[4-({[(3-chlorophenyl)amino]carbonyl}amino)phenyl)pyridin-2-yl}carbonyl)cyclopentanecarboxylic acid;

Trans-2-({5-[4-({[(2-fluorophenyl)amino]carbonyl}amino)phenyl]pyridin-2-yl}carbonyl)cyclopentanecarboxylic acid;

Trans-2-[(5-{4-[({[2-fluoro-5-(trifluoromethyl)phenyl]amino}carbonyl)amino]phenyl}pyridin-2-yl)carbonyl]cyclopentanecarboxylic acid;

Trans-2-[(5-{4-[(phenylacetyl)amino]phenyl}pyridin-2-yl)carbonyl]cyclopentanecarboxylic acid;

Trans-2-{[5-(4-{[(2-ethoxyphenyl)acetyl]amino}phenyl)pyridin-2-yl]carbonyl}cyclopentanecarboxylic acid;

Trans-2-{[5-(4-{[(3,5-dimethylphenyl)acetyl]amino}phenyl)pyridin-2-yl]carbonyl}cyclopentanecarboxylic acid;

Trans-2-{[5-(4-{[(2R)-2-phenylpropanoyl]amino}phenyl)pyridin-2-yl]carbonyl}cyclopentanecarboxylic acid;

Trans-2-{[5-(4-{[fluoro(phenyl)acetyl]amino}phenyl)pyridin-2-yl]carbonyl}cyclopentanecarboxylic acid;

Trans-2-[(5-{4-[(thien-3-ylacetyl)amino]phenyl}pyridin-2-yl)carbonyl]cyclopentanecarboxylic acid;

Trans-2-[(5-{4-[(pyridin-3-ylacetyl)amino]phenyl}pyridin-2-yl)carbonyl]cyclopentanecarboxylic acid;

Trans-2-{[5-(4-{[(1-phenylcyclopropyl)carbonyl]amino}phenyl)pyridin-2-yl]carbonyl}cyclopentanecarboxylic acid;

Trans-2-[(5-{4-[(anilinocarbonyl)amino]-3-fluorophenyl}pyridin-2-yl)carbonyl]cyclopentanecarboxylic acid;

Trans-2-[(5-{3-fluoro-4-[(phenylacetyl)amino]phenyl}pyridin-2-yl)carbonyl]cyclopentanecarboxylic acid;

Trans-2-({6′-[({[3-(trifluoromethyl)phenyl]amino}carbonyl)amino]-3,3′-bipyridin-6-yl}carbonyl)cyclopentanecarboxylic acid;

Trans-N-[2-fluoro-5-(trifluoromethyl)phenyl]-N′-[4-(2-{[(1S,2S)-2-(1-hydroxy-1-methylethyl)cyclopentyl]carbonyl}-1,3-thiazol-5-yl)phenyl]urea;

Trans-2-[(5-{4-[({[2-fluoro-5-(trifluoromethyl)phenyl]amino}carbonyl)amino]phenyl}thien-2-yl)carbonyl]cyclobutane carboxylic acid;

Trans-2-[(5-{4-[({[2-fluoro-5-(trifluoromethyl)phenyl]amino}carbonyl)amino]phenyl}-1,3-thiazol-2-yl)carbonyl]cyclopentanecarboxylic acid;

Trans-2-[(5-{3-fluoro-4-[({[3-(trifluoromethyl)phenyl]amino}carbonyl)amino]phenyl}-1,3-thiazol-2-yl)carbonyl]cyclopentanecarboxylic acid;

Trans-2-[(5-{3-fluoro-4-[({2-fluoro-5-(trifluoromethyl)phenyl]amino}carbonyl)amino]phenyl}-1,3-thiazol-2-yl)carbonyl]cyclopentanecarboxylic acid;

Trans-2-[(5-{4-[({[2-fluoro-5-(trifluoromethyl)phenyl]amino}carbonyl)amino]phenyl}-1,3-thiazol-2-yl)carbonyl]cyclobutane carboxylic acid; and

Trans-2-[(5-{3-fluoro-4-[({[3-(trifluoromethyl)phenyl]amino}carbonyl)amino]phenyl}thien-2-yl)carbonyl]cyclobutane carboxylic acid;

or a pharmaceutically acceptable salt, prodrug, or salt of a prodrug thereof.

Compounds disclosed herein can contain asymmetrically substituted carbon or sulfur atom, and accordingly can exist in, and be isolated in, single stereoisomers (e.g. single enantiomer or single diastereomer), mixtures of stereoisomers (e.g. any mixture of enantiomers or diastereomers) or racemic mixtures thereof. Individual optically-active form of the compounds can be prepared for example, by synthesis from optically-active starting materials, by chiral synthesis, by enzymatic resolution, by biotransformation, or by chromatographic separation. It is to be understood that the present invention encompasses any racemic, optically-active, stereoisomeric form, or mixtures of various proportions thereof, which form possesses properties useful in the inhibition of DGAT-1 activity. Where the stereochemistry of the chiral centers present in the chemical structures illustrated herein is not specified, the chemical structure is intended to encompass compounds containing either stereoisomer of each chiral center present in the compound.

Examples of some of the possible stereoisomers of the compounds of this invention are represented by formula (If) and (Ig). It is understood that the structural drawing of (If) encompasses not only one particular trans isomer as depicted in (If), but also other trans isomers (for example, (Ig)), racemates and mixtures of various proportions of (If) and (Ig) 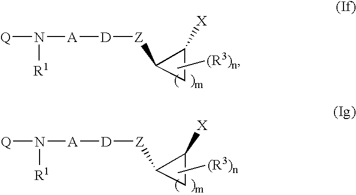
wherein R¹, R³, X, Q, Z, A, D, m, and n are as described in the summary of the invention.

It is understood that particular values of the variable groups (for example, R¹, R³, X, Q, Z, A, D, m, and n), and combinations of embodiments, including preferred, more preferred and most preferred embodiments as described in formula (I) are also contemplated for compounds of formulae (If) and (Ig).

Geometric isomers can exist in the present compounds. The invention contemplates the various geometric isomers and mixtures thereof resulting from the disposition of substituents around a carbon-carbon double bond, a carbon nitrogen double bond, a cycloalkyl group, or a heterocycloalkyl group. Substituents around a carbon-carbon or carbon-nitrogen double bond are designated as being of Z or E configuration and substituents around a cycloalkyl or heterocycloalkyl are designated as being of cis or trans configuration.

Within the present invention it is to be understood that compounds disclosed herein can exhibit the phenomenon of tautomerism.

Thus, the formulae drawings within this specification can represent only one of the possible tautomeric or stereoisomeric forms. It is to be understood that the invention encompasses any tautomeric or stereoisomeric form, and mixtures thereof, and is not to be limited merely to any one tautomeric or stereoisomeric form utilized within the naming of the compounds or formulae drawings.

Synthetic Methods

This invention is intended to encompass compounds of the invention when prepared by synthetic processes or by metabolic processes. Preparation of the compounds of the invention by metabolic processes include those occurring in the human or animal body (in vivo) or processes occurring in vitro.

The synthesis of compounds of formula (I), (Ia), (Ib), (Ic), (Id), (Ie), (If), or (Ig), wherein the groups R¹, R², R^{2a}, R³, R⁵, R⁶, R⁷, R^{b}, Q, X, Z, A, D, m, and n have the meanings as set forth in the summary section unless otherwise noted, is exemplified in Schemes 1-10

As used in the descriptions of the schemes and the examples, certain abbreviations are intended to have the following meanings: DMSO for dimethylsulfoxide and RP-HPLC for preparative reverse phase high pressure liquid chromatography.

Compounds of general formula (I), (Ia), (Ib), (Ic), (Id), (Ie), (If) or (Ig), wherein Z is C(O) prepared can be prepared using the general procedures as outlined in Scheme 1.

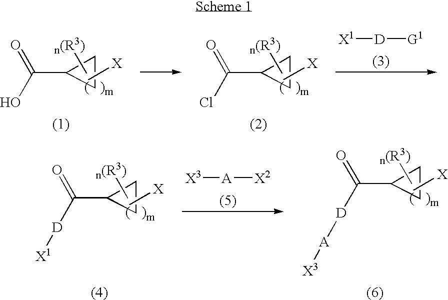

As illustrated in Scheme 1, acids of formula (1) can react with a chlorinating agent in a solvent such as, but not limited to, dichloromethane, at a temperature from about room temperature to about 50° C., to provide acid chlorides of formula (2). Non-limiting examples of the chlorinating agents include phosphorus pentachloride, oxalyl chloride, and thionyl chloride with or without catalytic N,N-dimethylformamide. The acid chloride of formula (2) can be treated with compounds of formula (3) wherein X¹ is halogen or triflate, G¹ is hydrogen, and D is phenyl or heteroaryl, in the presence of Lewis acids such as, but not limited to, aluminum chloride, in a solvent such as, but not limited to, dichloromethane, or dichloroethane to provide a compound of formula (4). In some instances, the compound of formula (3) can act as the reactant as well as the reaction solvent. The reaction is generally conducted at a temperature ranging from about 0° C. to about 10° C.

Alternatively, compounds of formula (4) can be prepared by reacting compounds of formula (2) with compounds of formula (3) wherein G¹ is a reactive substituent such as, but not limited to, —ZnI, —B(OR₁₀₁)₂ wherein R₁₀₁ is hydrogen or C₁₋₆ alkyl, in the presence of a palladium catalyst.

Compounds of formula (4) can be treated with boronic acids or esters of formula (5) wherein A is phenyl or heteroaryl, X³ is NO₂, N(H)(P_{G}) wherein P_{G} is an amine protecting group, or R²N(R¹)C(═Y)N(R^{2a}), and X² is —B(OR₁₀₁)₂ wherein R₁₀₁ is hydrogen or C₁₋₆ alkyl, in the presence of a palladium catalyst, in a solvent such as, but not limited to, toluene, dioxane, N,N-dimethylformamide, N,N-dimethyl acetamide, dimethoxyethane, dimethylsulfoxide, isopropanol, ethanol, water, or mixture thereof, to provide compounds of formula (6). Non-limiting examples of palladium catalyst suitable for the transformation include, tetrakis(triphenylphosphine)palladium(0), bis(triphenylphosphine)palladium (II) chloride, and [1,1′-bis(diphenylphosphino)ferrocene]dichloropalladium (II). The reaction can be facilitated with the addition of a base such as, but not limited to, potassium iodide, triethylamine, cesium carbonate, sodium carbonate, potassium phosphate, potassium fluoride, or thallium ethoxide. The reaction is generally conducted at an elevated temperature such as 50° C. to about 100° C. and optionally in a microwave oven.

Compounds of formula (4) can also be treated with stannanes of formula (5) wherein A is phenyl or heteroaryl, X³ is as defined hereinabove, and X² is —Sn(C₁₋₆ alkyl)₃, in the presence of a palladium catalyst such as, but not limited to, tetrakis(triphenylphosphine)palladium (0), and heating, in a solvent such as dioxane, to provide compounds of formula (6). The transformations can also be effected by heating in a microwave reactor.

Alternatively, compounds of formula (4) wherein D is phenyl or heteroaryl, and X¹ is B(OR₁₀₁)₂ or Sn(C₁₋₆ alkyl)₃, can be treated with compounds of formula (5) wherein A is phenyl or heteroaryl, X² is halogen or triflate, and X³ is as defined hereinabove, using reaction conditions as described in the preceding paragraphs to provide compounds of formula (6). While many stannanes and boronic acids or esters are commercially available, compounds of formula (4) and (5) wherein X¹ and X² are independently B(OR₁₀₁)₂ or Sn(C₁₋₆ alkyl)₃ can also be prepared by treating the corresponding halides or triflates, with boronate esters of formula (R₁₀₁O)₂B—B(OR₁₀₁)₂ or distannanes of formula ((C₁₋₆ alkyl)₃Sn)₂, in the presence of a palladium catalyst using methodologies that are known in the art.

Compounds of formula (6) can also be prepared from treatment of compounds of formula (2) with compounds of formula X³-A-D-G¹ wherein X³ is NO₂, N(H)(P_{G}) and P_{G} is an amine protecting group, or R²N(R¹)C(═Y)N(R¹), and G¹ is hydrogen, ZnI or —B(R₁₀₁)₂ wherein R₁₀₁ is hydrogen or C₁₋₆ alkyl, using reaction conditions as described above for the transformation of (2) to (4).

Scheme 2 illustrates general procedure for the synthesis of compounds of general formula (I) wherein Z is C(O) and Q is —C(═O)N(R^{2a})(R²), —C(═S)N(R^{2a})(R²), or —C(═O)(R^{b})

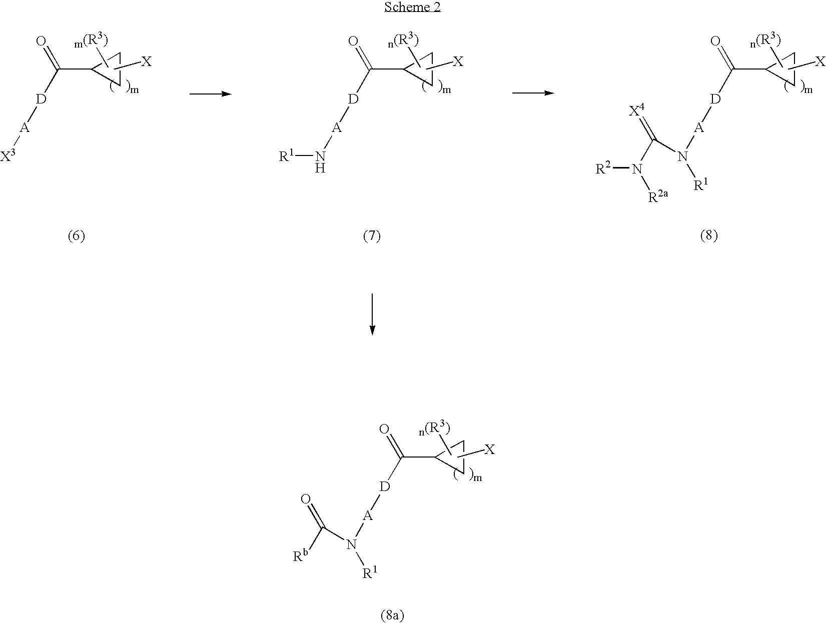

Compounds of formula (6) wherein X³ is NO₂ can be converted to compounds of formula (7) wherein R¹ is hydrogen, by treatment with a reducing agent in a suitable solvent. Examples of reducing agents suitable for the conversion include, but not limited to, iron in the presence of an acid (for example, acetic acid, ammonium chloride, and the like), or hydrogen gas and palladium catalyst (egg 5-10% palladium on carbon, and 20% palladium hydroxide on carbon) Compounds of formula (7) can also be obtained by deprotection of compounds of formula (6) when X³ is N(R¹)P_{G} by means well known in the art. Compounds of formula (8) wherein R^{2a}; is hydrogen, X⁴ is O or S can be prepared by reaction of compounds of formula (7) with isocyanates or isothiocyanates of formula R²NCX⁴, in a solvent such as, but not limited to, tetrahydrofuran, at about room temperature. Compounds of formula (8) wherein R^{2a} is lower alkyl can be prepared from compounds of formula (8) wherein R^{2a} is hydrogen by treatment with an alkylating agent of formula (C₁₋₆ alkyl)-X^{a} wherein X^{a} is halide, triflate or alkyl sulfonates or aromatic sulfonates such as p-toluenesulfonate, in the presence of a base, in a solvent. Non limiting examples of suitable bases include organic bases (for example trialkylamines such as triethylamine, diisopropylethylamine and the like), pyridine, picoline, or tertiary cyclic amines such as N-methylmorpholine) and inorganic bases (for example, alkali metal hydrides, alkali metal hydroxides, alkali metal carbonates and alkali metal bicarbonates).

Compounds of formula (8a) wherein R¹ is hydrogen can be obtained from their reaction of compounds of formula (7a) with an acid of formula R^{b}C(O)OH using coupling reaction conditions known to one skilled in the art. Compounds of formula (7a) wherein R¹ is H can be converted to compounds of formula (7a) wherein R¹ is alkyl by treatment with an alkylating agent as described in the preceding paragraph.

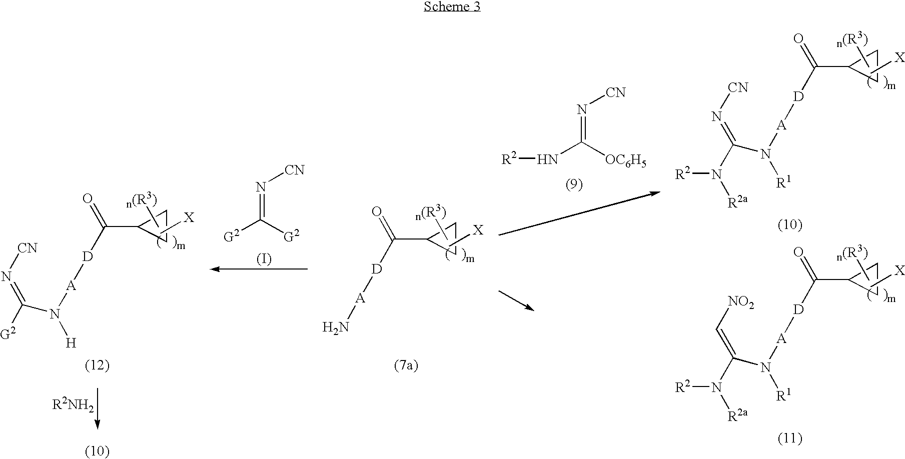

Compounds of formula (10) wherein R^{2a} and R¹ are hydrogen can be prepared from compounds of formula (7a) by treating with compounds of formula (9) in a solvent such as, but not limited to, acetonitrile, at elevated temperature (for example, 70-150° C., more generally at about 140° C.) in a microwave oven. Compounds of formula (9) can be obtained from reaction of diphenyl cyanocabonimidate with amines of formula R²NH₂, in a solvent such as, but not limited to, acetonitrile. Compounds of formula (11) wherein R^{2a} and R¹ are hydrogen can be obtained by (a) refluxing (7a) with 1,1-bis(methylthio)-nitroethylene, and (b) treating the product from step (a) with amines of formula R²NH₂ at room temperature, followed by heating at about 60° C. until the reaction is complete.

Alternatively, compounds of formula (10) wherein R^{2a} is hydrogen can be prepared by (a) treating compounds of formula (7a) with compounds of formula (i) wherein G² is SCH₃ or O(C₆H₅), and (b) treating compounds of formula (12) obtained from step (a) with amines of formula R²NH₂.

Both compounds of formula (10) and (11) wherein R^{2a} and R¹ are hydrogen can be alkylated to provide compounds of formula (10) and (11) wherein R^{2a} and R¹ are lower alkyl, using the alkylation reaction conditions as described in Scheme 2 and an appropriate alkylating reagent.

Compounds of general formula (I) wherein Z is C(O) and X is —C(R⁶R⁷)OH can be synthesized using general procedures as outlined in Scheme 4.

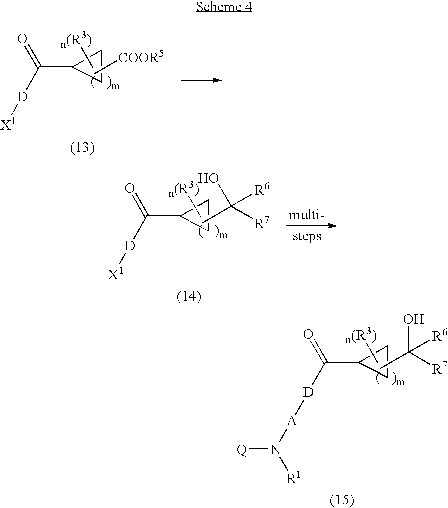

Compounds of formula (13) can be converted to compounds of formula (14) wherein R⁶ and R⁷ are the same and both are alkyl by (a) selectively reducing the carbonyl functionality between D and the cycloalkyl group with a reducing agent such as sodium borohydride at temperature of about 0° C., (b) treating the intermediate from step (a) with about two equivalents of the Grinard reagent of formula R⁶MgX⁵ wherein X⁵ is Cl, Br or I, and (c) treating the intermediate from step (b) with an oxidizing agent such as, but not limited to, pyridinium chlorochromate. Transformation of compounds of formula (14) using reaction conditions as described in Schemes 1, 2 and 3, provides compounds of formula (15).

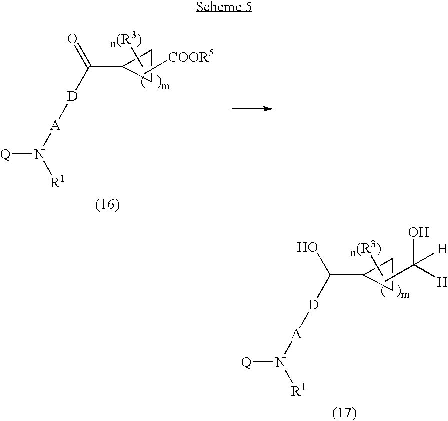

Reduction of compounds of formula (16) to provide compounds of formula (17) can be accomplished by treatment with a reducing agent such as, but not limited to, sodium borohydride or lithium aluminum hydride, in a solvent such as tetrahydrofuran, at a temperature from about 0° C. to about room temperature. The diastereomers obtained after isolation can be separated using techniques known in the art, for example, silica gel chromatography.

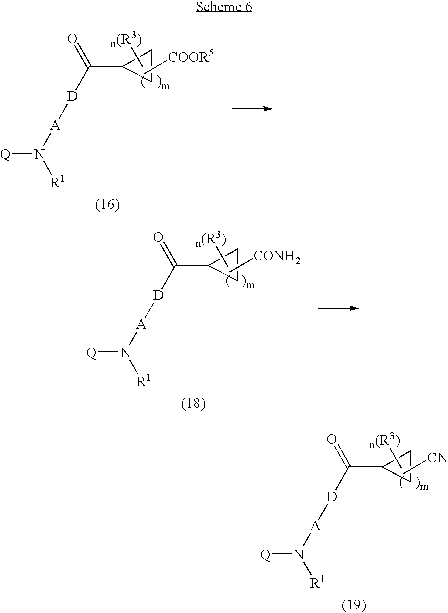

Conversion of compounds of formula (16) to amides of formula (18) can be achieved by (a) hydrolyzing compounds of formula (16) wherein R⁵ is alkyl, (b) converting the corresponding acids of formula (16) wherein R⁵ is hydrogen to an activated ester, for example, by treatment with N-hydroxy succinamide, N-(3-dimethylaminopropyl)-N′-ethylcarbodiimide hydrochloride and a base such as, N-methyl morpholine, in a solvent such as, dichloromethane, and (c) generally without isolation of the activated ester from step (b), treating the activated ester with ammonia. The ammonia source used can be ammonium chloride, gaseous ammonia, or ammonia in a suitable solvent such as alcohol, water or dioxane.

Treatment of compounds of formula (18) with a dehydrating agent in a suitable solvent provides compounds of formula (19). Non limiting examples of dehydrating agent are phosphorous pentoxide, phosphoryl chloride/pyridine or imidazole, trifluoroacetic anhydride/pyridine, and thionyl chloride.

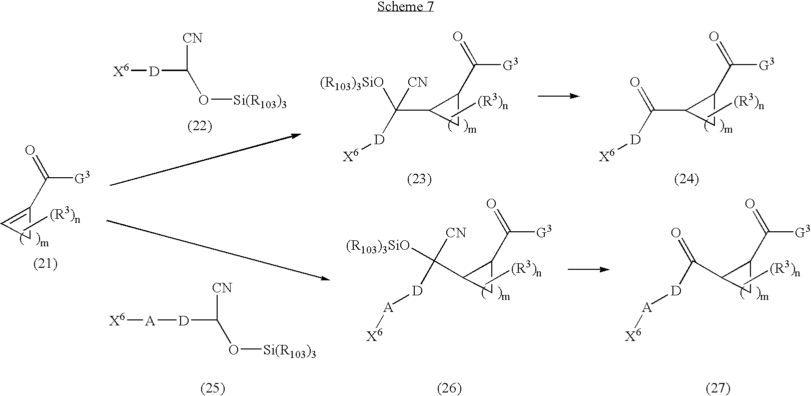

Compounds of formula (24) or (27) wherein G³ is —O(alkyl) or allyl, can be prepared from cycloalkenes of formula (21) as illustrated in Scheme 7.

Treatment of compounds of formula (21) with compounds of formula (22) or (25) wherein X⁶ is hydrogen or halides, and each R₁₀₃ can be the same or different C₁₋₆ alkyl, in the presence of a strong base such as, but not limited to, lithium diisopropylamide, provides compounds of formula (23) or (26) respectively. Compounds of formula (22) ox (25) can be obtained from the corresponding aldehydes by treatment with potassium cyanide and trialkyl silyl halides of formula (R₁₀₃)₃SiX⁷ wherein X⁷ is halogen.

Reaction of compounds of formula (23) and formula (26) with tetrabutyl ammonium fluoride in acidic conditions (such as in the presence of acetic acid) provides compounds of formula (24) and (27) respectively. If desired, compounds of formula (24) and (27) can be resolved into their respective diastereomers using standard means, for example, via silica gel column chromatography.

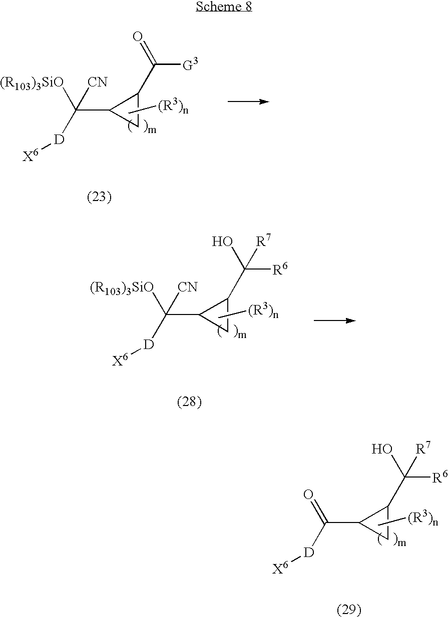

Compounds of formula (23) (prepared from Scheme 7) wherein G³ is —O(alkyl) or alkyl, can be treated with a reducing agent such as sodium borohydride to provide alcohols of formula (28) wherein R⁶ and R⁷ are both hydrogen. Compounds of formula (28) wherein R⁶ and R⁷ are the same and are alkyl, can be obtained from reaction of (23) with about one equivalent of Grignard reagent of formula R⁶MgX⁵ when G³ is alkyl, or with at least two equivalents of Grignard reagent of formula R⁶MgX⁵ when G³ is —O(alkyl).

Utilizing the reaction conditions as described in Scheme 7 for the transformation of (23) to (24) converts compounds of formula (28) to (29).

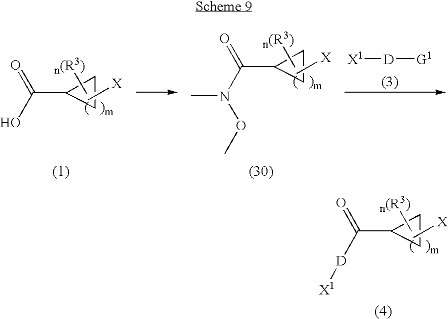

Scheme 9 illustrates an alternative method of synthesis for compounds of formula (4) wherein X¹ is bromide.

Compounds of formula (1) can be transformed into compounds of formula (30) when treated with a reagent such as, but not limited to, N,O-dimethylhydroxylamine hydrochloride or morpholine, and a coupling reagent such as, but not limited to, 1-ethyl-3-(3-dimethylaminopropyl)carbodiimide, and in the presence of an auxiliary nucleophile such as, but not limited to, hydroxybenzotriazole, and in the presence of a base such as, but not limited to, N-methyl morpholine, and in a solvent such as, but not limited to, N,N-dimethylformamide. The reaction can be conducted at room temperature. The amide of formula (30) can be treated with compounds of formula (3) wherein X¹ is bromide, G¹ is halide, and D is phenyl or heteroaryl, in the presence of a Grignard reagent such as isopropylmagnesium chloride, or an alkyl lithium reagent, such as, but not limited to, n-butyllithium, in a solvent such as, but not limited to, tetrahydrofuran or ether, to provide a compound of formula (4). The reaction is generally conducted at a temperature ranging from about −20° C. to about 10° C.

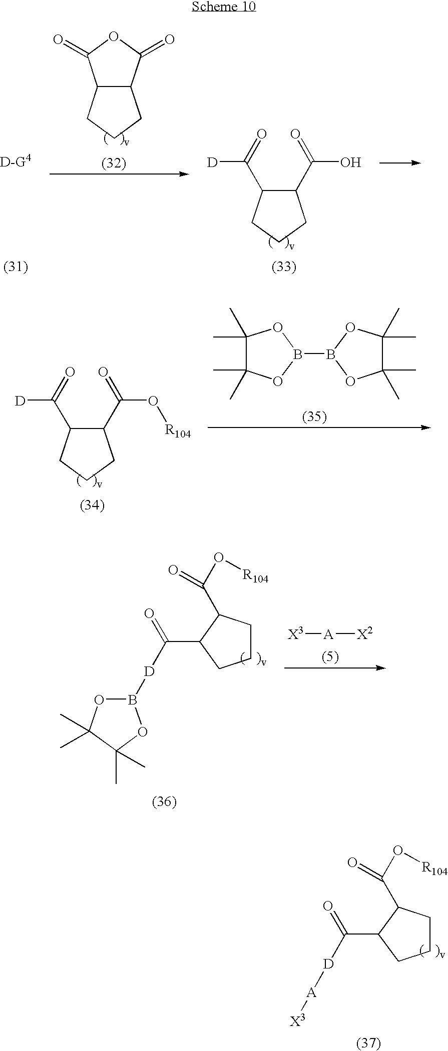

As illustrated in Scheme 10, compounds of formula (31) wherein G⁴ is a reactive substituent, such as but not limited to —ZnBr, and D is heteroaryl, can react with anhydrides of formula (2) wherein v=0, 1, 2, or 3 in the presence of a palladium catalyst, in a solvent such as, but not limited to, tetrahydrofuran, dimethoxyethane or the like, at a temperature from about room temperature to about 90° C., to provide compounds of formula (33). Compounds of formula (33) can be treated with an alkylating agent such as, but not limited to, methyl iodide, in the presence of acid such as, but not limited to, potassium carbonate, and in a solvent such as, but not limited to, N,N-dimethylformamide, to afford esters of formula (34), wherein R₁₀₄ is alkyl. The reaction can be run at room temperature to about 70° C.

Compounds of formula (33) or (34) can be treated with 4,4,4′,4′,5,5,5′,5′-octamethyl-2,2′-bi(1,3,2-dioxaborolane) in the presence of an iridium catalyst and bipyridine ligand, in a solvent such as, but not limited hexane, octane, mesitylene, toluene, of xylenes, at a temperature from about 80° C. to about 120° C., to provide compounds of formula (36) wherein R₁₀₄ is hydrogen or alkyl. Non-limiting examples of catalysts suitable for the transformation include methoxybis(1,5-cyclooctadiene)iridium(I) dimer and chlorobis(cyclooctene)iridium(I) dimer. Non-limiting examples of bipyridine ligands suitable for the transformation include 4,4′-di-t-butyl-2,2′-bipyridine, 3,3,-dimethyl-bipyridine, 4,4′-dimethyl-bipyridine, 5,5′-dimethyl-bipyridine, and 4,4′-dimethoxy-bipyridine.

Compounds of formula (36) wherein R₁₀₄ is hydrogen or alkyl can be treated with compounds of formula (5) wherein A is phenyl or heteroaryl, X³ is NO₂, N(H)(P_{G}) and P_{G} is an amine protecting group, or R²N(R^{2a})C(═Y)N(R¹), and X² is halide or triflate using reaction conditions as described in Scheme 1, to provide compounds of formula (37).

It is appreciated that the synthetic schemes and specific examples as illustrated in the synthetic examples section are illustrative and are not to be read as limiting the scope of the invention as it is defined in the appended claims All alternatives, modifications, and equivalents of the synthetic methods and specific examples are included within the scope of the claims

Optimum reaction conditions and reaction times for each individual step can vary depending on the particular reactants employed and substituents present in the reactants used. Unless otherwise specified, solvents, temperatures and other reaction conditions can be readily selected by one of ordinary skill in the art. Specific procedures are provided in the Synthetic Examples section Reactions can be worked up in the convention manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature.

Routine experimentations, including appropriate manipulation of the reaction conditions, reagents and sequence of the synthetic route, protection of any chemical functionality that can not be compatible with the reaction conditions, and deprotection at suitable point in the reaction sequence of the method are included in the scope of the invention. Suitable protecting groups and the methods for protecting and deprotecting different substituents using such suitable protecting groups are well known to those skilled in the art; examples of which can be found in T. Greene and P. Wuts, Protecting Groups in Chemical Synthesis (3^{rd} ed.), John Wiley & Sons, NY (1999), which is incorporated herein by reference in its entirety. Synthesis of the compounds of formula (I), (Ia), (Ib), (IIa) or (IIb) can be accomplished by methods analogous to those described in the synthetic schemes described hereinabove and in specific examples.

Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

When an optically active form of a compound of the invention is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound of the invention is required, it can be obtained by carrying out one of the above procedures using a pure geometric isomer as a starting material, or by resolution of a mixture of the geometric isomers of the compound or intermediates using a standard procedure such as chromatographic separation.

Biological Data

Inhibition of DGAT-1

The identification of the compounds of the invention as DGAT-1 inhibitors was readily achieved using a high throughput screening FlashPlate assay. In this assay, recombinant human DGAT-1 containing an N-terminal His₆-epitope tag was produced in the baculovirus expression system. Insect cells (e.g., Sf9 or High Five) were infected for 24 to 72 hours and collected by centrifugation. Cell pellets were resuspended in homogenization buffer [250 mM sucrose, 10 mM Tris-HCl (pH 7.4), 1 mM EDTA and lysed using a homogenization apparatus, such as a Microfluidizer (single pass, 4° C.). Cell debris was removed by centrifugation at 10,000×g for 30 min, and microsomal membranes were collected by ultracentrifugation at 100,000×g for 30 min.

DGAT-1 activity was determined as follows: Assay buffer [20 mM HEPES (pH 7.5), 2 mM MgCl₂, 0.04% BSA] containing 50 μM of enzyme substrate (didecanoyl glycerol) and 7.5 μM radiolabeled acyl-CoA substrate [1-¹⁴C]decanoyl-CoA) was added to each well of a phospholipid FlashPlate (PerkinElmer Life Sciences). A small aliquot of membrane (1 μg/ell) was added to start the reaction, which was allowed to proceed for 60 min. The reaction was terminated upon the addition of an equal volume (100 μL) of isopropanol. The plates were sealed, incubated overnight and counted the next morning on a TopCount Scintillation Plate Reader (PerkinElmer Life Science). DGAT-1 catalyzes the transfer of the radiolabel-led decanoyl group onto the s7i-3 position of didecanoyl glycerol. The resultant radiolabeled tridecanoyl glycerol (tricaprin) preferentially binds to the hydrophobic coating on the phospholipid FlashPlate. The proximity of the radiolabeled product to the solid scintillant incorporated into the bottom of the FlashPlate induced fluor release from the scintillant, which was measured in the TopCount Plate Reader. Various concentrations (e.g. 00001 μM, 0.001 μM, 0.01 μM, 0.1 μM, 1.0 μM, 10.0 μM) of the representative compounds of the invention were added to individual wells prior to the addition of membranes. The potencies of DGAT-1 inhibition for the compounds of the present invention were determined by calculating the IC₅₀ values defined as the inhibitor concentration from the sigmoidal dose response curve at which the enzyme activity was inhibited 50%. Compounds of the present invention were effective in inhibiting DGAT-1 activity and thus are useful as therapeutic agents for treating conditions and diseases that are associated with DGAT-1 activity.

TABLE 1
DGAT-1 Inhibition of compounds of
the present invention (IC₅₀ nM)

| Compound | IC₅₀ μM |
|---|---|
| A | 0.05148 |
| B | 1.06856 |
| c | 0.01899 |
| D | 0.01325 |
| E | 0.0124 |
| F | 4.54985 |
| G | 0.0308 |
| H | 3.2266 |
| I | 0.19972 |
| J | 1.06588 |
| K | 0.03874 |
| L | 0.256 |
| M | 0.32788 |
| N | 0.32156 |
| O | 0.87774 |
| P | 0.35229 |
| Q | 9.63071 |
| R | 0.58 |
| S | 0.03435 |
| T | 0.60852 |

Evaluation of Compound Efficacy on the Reduction of Body Weight in Diet-Induced Obese Mice

The purpose of this protocol was to determine the effect of chronic administration of a compound on body weight and other metabolic disease parameters in mice made obese by spontaneous ad libitum consumption of a high-fat diet. Diet-induced obesity (DIO) in rodents mimics key aspects of human obesity and metabolic syndrome. DIO mice used in this study have been shown to be hyperinsulinemic and insulin resistant, hyperleptinemic and leptin resistant, and have marked visceral obesity (for review on DIO mice see Collins et al., Physiol. Behav. 81:243-248, 2004).

Individually housed male C57BL/6J mice were given ad lib access to water and to either a low fat diet (D12450B) or a high-fat content diet (D12492 containing 60% kcal from fat, both from Research Diets Inc., New Brunswick, N.J.), for approximately 18 weeks. Mice were sham dosed once daily with the study vehicle for 7 days prior to active dosing to acclimate them to handling and oral gavage. One day prior to active compound dosing, mice were assigned to groups of equal mean body weight and variance. A typical experiment consisted of 80-100 animals, 10 animals pet dose including vehicle dosed low-fat and high-fat diet groups. Body weight and food intake were measured by differential weighing.

Representative compounds of the invention were typically dosed at 3, 10, or 30 mg/kg p.o. b.i.d. as a formulation in 1% Tween 80 in water, and the compounds were considered to be active if a statistically significant reduction in body weight was observed for the treated animals after a treatment period of at least seven days, relative to vehicle-treated control animals. In this model, representative compounds produced a statistically significant reduction in body weight after a treatment period of at least seven days, relative to vehicle-treated control animals.

Liver triacylglycerides levels from DIO-mice treated with compounds of the invention typically dosed at 3, 10, or 30 mg/kg p.o. b.i.d. as a formulation in 1% Tween 80 in water for a treatment period of at least seven days were measured from ethanol extracted liver samples using Infinity TM reagents (Thermo Electron Corporation, Louisville, Colo., USA). Representative compounds of the invention produced a statistically significant reduction in liver triacylglycerides in DIO-mice after a treatment period of at least seven days, relative to vehicle-treated control animals.

An insulin tolerance test was also performed at the end of study in DIO mice after a 4 hour fast. Blood glucose levels were monitored via tail snip before and at 30 minute intervals following a single i.p. injection of 0.25 U/kg insulin (Humulin-R, Lilly) using a Precision PCx glucose monitor (Abbott Laboratories, Abbott Park, Ill.) Representative compounds of the invention produced a statistically significant reduction in blood glucose in animals that had been treated for at least seven days, relative to vehicle-treated control animals.

The effect of co-dosing representative compounds of the invention with rimonabant was also evaluated in DIO-mice. Compounds of the invention were typically dosed at 3, 10, or 30 mg/kg p.o. b.i.d. as a formulation in 1% Tween 80 in water and rimonabant was typically co-administered at a dose of 3 or 10 mg/kg p.o. q.d. as a formulation in 1% Tween in water. Compounds were considered to be active if they significantly decreased body weight compared to DIO-nice dosed with rimonabant alone. In this model, representative compounds produced a statistically significant reduction in body weight after a treatment period of at least seven days, relative to animals treated with rimonabant alone.

The effect of co-dosing representative compounds of the invention with fenofibrate was also evaluated in DIO-mice. Compounds of the invention were typically dosed at 3, 10, or 30 mg/kg p.o. b.i.d. as a formulation in 1% Tween 80 in water and fenofibrate was typically co-administered at a dose of 100 mg/kg p.o. b.i.d. as a formulation in 1% Tween in water. Compounds were considered to be active if they significantly decreased body weight compared to DIO-nice dosed with fenofibrate alone. In this model, representative compounds produced a statistically significant reduction in body weight after a treatment period of at least seven days, relative to animals treated with fenofibrate alone

Compounds of the present invention and the pharmaceutically acceptable salts are useful as therapeutic agents. Accordingly, an embodiment of this invention includes a method of treating the various conditions in a subject in need thereof (including mammals) which includes administering to the subject a pharmaceutical composition containing an amount of the compound of formula (I), (Ia), (Ib), (Ic), (Ic), (Ie), (If), or (Ig), that is effective in treating the target condition, or a pharmaceutically acceptable salt thereof, together with a pharmaceutically acceptable carrier.

Another aspect of the present invention provides a method of treating, delay or prevention of various conditions in a patient (such as mammal, preferably human) that are mediated by DGAT-1, which includes administering to the patient a compound of formula (I), (Ia), (Ib), (Ic), (Id), (Ie), (If), or (Ig), or a pharmaceutically acceptable salt, prodrug, salt of a prodrug, or a combination thereof, or a pharmaceutical composition including the same.

Another aspect of the present invention provides methods for the prevention, delay or treatment of obesity and inducing weight loss in an individual which includes administering to the individual a compound of the invention, or its pharmaceutically acceptable salt, prodrug, salt of a prodrug, or a combination thereof. The invention further provides a method for the prevention, delay or treatment of obesity and inducing weight loss in an individual which includes administering to the individual a pharmaceutical composition including a compound of the invention, or its pharmaceutically acceptable salt, prodrug, salt of a prodrug, or a combination thereof, in an amount that is effective in treating obesity or to induce weight loss, and a pharmaceutically acceptable carrier. Yet another aspect of the invention provides a method for preventing weight gain in an individual by administering at least one compound of the invention, or its pharmaceutically acceptable salt, prodrug, salt of a prodrug, or a combination thereof, in an amount that is sufficient to prevent weight gain.

The present invention also relates to the use of the compounds of this invention for the treatment of obesity-related diseases including associated dyslipidemia and other obesity- and overweight-related complications such as, for example, cholesterol gallstones, gallbladder disease, gout, cancer (e.g., colon, rectum, prostate, breast, ovary, endometrium, cervix, gallbladder, and bile duct), menstrual abnormalities, infertility, polycystic ovaries, osteoarthritis, and sleep apnea, as well as for a number of other pharmaceutical uses associated therewith, such as the regulation of appetite and food intake, dyslipidemia, hypertriglyceridemia, metabolic syndrome or Syndrome X, type 2 diabetes (non-insulin-dependent diabetes), atherosclerotic diseases such as heart failure, hyperlipidemia, hypercholesteremia, low HDL levels, hypertension, cardiovascular disease (including atherosclerosis, coronary heart disease, coronary artery disease, and hypertension), cerebrovascular disease such as stroke, and peripheral vessel disease. The compounds of this invention can also be useful for treating physiological disorders related to, for example, regulation of insulin sensitivity, inflammatory response, liver steatosis, elevated liver triacylglycerides, non-alcoholic fatty liver disease, nonalcoholic steatohepatitis, plasma triacylglycerides, HDL, LDL and cholesterol levels and the like. Metabolic syndrome is characterized by a group of metabolic risk factors in one person. Such factors include, but are not limited to, abdominal obesity, atherogenic dyslipidemia (blood flat disorders such as high triglycerides, low HDL cholesterol and high LDL cholesterol), elevated blood pressure, insulin resistance (or glucose intolerance), prothrombolic state (e.g., high fibrinogen or plasminogen activator inhibitor-1 in the blood), and proinflammatory state (e.g. elevated C-reactive protein in the blood). In one embodiment, the present invention provides methods of treating the above listed disorders wherein the methods include the step of administering to a subject in need thereof a compound of the invention, or a pharmaceutically acceptable salt thereof, or a pharmaceutical composition including the same. The compounds of this invention, or pharmaceutical acceptable salts thereof, or pharmaceutical compositions including the same, are also useful in lowering plasma triglycerides level. Thus, in one embodiment, the present invention provides a method for lowering plasma triglycerides in a subject (including mammal) in need thereof, wherein the method includes the step of administering to the subject in need thereof a compound of the invention, or a pharmaceutically acceptable salt thereof, or a pharmaceutical composition including the same.

Compounds of the invention or pharmaceutically acceptable salts thereof, can be administered alone or in combination (i.e. co-administered) with one or more additional pharmaceutical agents. Combination therapy includes administration of a single pharmaceutical dosage formulation which contains a compound of formula (I), (Ia), (Ib), (Ic), (Id), (Ie), (If), or (Ig), or a pharmaceutically acceptable salt thereof, and one or more additional pharmaceutical agents, as well as administration of the compound of formula (I), (Ia), (Ib), (Ic), (Id), (Ie), (If), or (Ig), or a pharmaceutically acceptable salt thereof, and each additional pharmaceutical agent, in its own separate pharmaceutical dosage formulation. For example, a compound of formula (I), (Ia), (Ib), (Ic), (Id), (Ie), (If), or (Ig), or a pharmaceutically acceptable salt thereof, and a pharmaceutical agent, can be administered to the patient together, in a single oral dosage composition having a fixed ratio of each active ingredient, such as a tablet or capsule, or each agent can be administered in separate oral dosage formulations.

Where separate dosage formulations are used, compounds of the invention and one or more additional pharmaceutical agents can be administered at essentially the same time (e.g., concurrently) or at separately staggered times (e.g., sequentially).

For example, the compounds of the invention can be used in combination with one of more of the following pharmaceutical agents, including, but are not limited to, anti-obesity drugs including β-3 agonists such as CL-316,243; CB-1 antagonists and/or inverse agonists (for example, rimonabant); neuropeptide Y5 inhibitors; appetite suppressants, such as, for example, sibutramine (Meridia® or Reductil®); MCHr1 antagonists and lipase inhibitors, such as, for example, orlistat (Xenical), and a drug compound that modulates digestion and/or metabolism such as drugs that modulate thermogenesis, lipolysis, gut motility, fat absorption, and satiety.

In addition, compounds of the invention can be administered in combination with one or more of the following pharmaceutical agents including PPAR ligands (agonists, antagonists), insulin secretagogues (for example, sulfonylurea drugs and non-sulfonylurea secretagogues), α-glucosidase inhibitors, insulin sensitizers, hepatic glucose output lowering compounds, and insulin and insulin derivatives. Such agents can be administered prior to, concurrently with, or following administration of the compounds of the invention. Insulin and insulin derivatives include both long and short acting forms and formulations of insulin. PPAR ligands can include agonists and/or antagonists of any of the PPAR receptors or combinations thereof. For example, PPAR ligands can include ligands of PPAR-α, PPAR-γ, PPAR-δ or any combination of two or three of the receptors of PPAR. PPAR ligands include, for example, rosiglitazone, troglitazone, and pioglitazone. Sulfonylurea drugs include, for example, glyburide, glimepiride, chlorpropamide, tolbutamide, and glipizide, α-glucosidase inhibitors include acarbose, miglitol, and voglibose. Insulin sensitizers include PPAR-γ agonists such as the glitazonies (e.g., troglitazone, pioglitazone, englitazone, MCC-555, rosiglitazone, and the like) and other thiazolidinedione and non-triazolidinedione compounds; biguanides such as metformin and pheniformin; protein tyrosine phosphatase-1B (PP-1B) inhibitors; dipeptidyl peptidase IV (DPP-IV) inhibitors, and 11beta-HSD inhibitors. Hepatic glucose output lowering compounds include glucagon antagonists and metformin, such as Glucophage and Glucophage XR. Insulin secretagogues include sulfonylurea and non-sulfonylurea drugs: CLP-1, GIP, PACAP, secretin, and derivatives thereof; nateglinide, meglitinide, repaglinide, glibenclamide, glimepiride, chlorpropamide, glipizide GLP-1 includes derivatives of GLP-1 with longer half-lives than native GLP-1, such as, for example, fatty-acid derivatized GLP-1 and exendin.

Compounds of the invention can also be used in methods of the invention in combination with one or more pharmaceutical agents including, but are not limited to, HMG-CoA red(Lictase inhibitors, nicotinic acid (for example, Niaspan®), fatty acid lowering compounds (e.g., acipimox); lipid lowering drugs (e.g., stanol esters, sterol glycosides such as tiqueside, and azetidinones such as ezetimibe), ACAT inhibitors (such as avasimibe), bile acid sequestrants, bile acid reuptake inhibitors, microsomal triacylglycerides transport inhibitors, and fibric acid derivatives HMG-CoA reductase inhibitors include, for example, statin such as lovastatin, simvastatin, pravastatin, fluvastatin, atorvastatin, rivastatin, itavastatin, cerivrastatin, and ZD-4522 Fibric acid derivatives include, for example, clofibrate, fenofibrate, bezafibrate, ciprofibrate, beclofibrate, etofibrate, and gemfibrozil. Sequestrants include, for example, cholestyramine, colestipol, and dialkylaminoalkyl derivatives of a cross-linked dextran.

Compounds of the invention can also be used in combination with anti-hypertensive drugs, such as, for example, β-blockers and ACE inhibitors. Examples of additional anti-hypertensive agents for use in combination with the compounds of the present invention include calcium channel blockers (L-type and T-type; e.g., diltiazem, verapamil, nifedipine, amlodipine and mybefradil), diuretics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthialidone, furosemide, musolimine, bumetanide, triamtrenene, amiloride, spironolactone), renin inhibitors, ACE inhibitors (e.g., captopril, zofenopril, fosinopril, enalapril, ceranopril, cilazopril, delapril, pentopril, quinapril, ramipril, lisinopril), AT-1 receptor antagonists (e.g., losartan, irbesartan, valsartan), ET receptor antagonists (e.g., sitaxsentan, atrsentan, neutral endopeptidase (NEP) inhibitors, vasopepsidase inhibitors (dual NEP-ACE inhibitors) (e.g., omapatrilat and gemopatrilat), and nitrates.

The compounds of this invention can also be co-administered with an incretin mimetic such as, but not limited to, exenatide.

The compounds of this invention can be utilized to achieve the desired pharmacological effect by administration to a subject in need thereof in an appropriately formulated pharmaceutical composition. A subject, for example, can be a mammal, including human, in need of treatment for a particular condition or disease. Therefore the present invention includes pharmaceutical compositions which include a therapeutically effective amount of a compound identified by the methods described herein, or a pharmaceutically acceptable salt thereof, in combination with a pharmaceutically acceptable carrier. The compounds identified by the methods described herein can be administered with a pharmaceutically acceptable carrier using any effective conventional dosage unit forms, for example, immediate and timed release preparations, orally, parenterally, topically, or the like.

The pharmaceutical compositions can be formulated for oral administration in solid or liquid form, for parenteral injection or for rectal administration.

Liquid dosage forms for oral administration of the present compounds include formulations of the same as emulsions, microemulsions, solutions, suspensions, syrups, and elixirs. In addition to the compounds, the liquid dosage forms can contain diluents and/or solubilizing or emulsifying agents. Besides inert diluents, the oral compositions can include wetting, emulsifying, sweetening, flavoring, and perfuming agents.

Injectable preparations of the present compounds include sterile, injectable, aqueous and oleaginous solutions, suspensions or emulsions, any of which can be optionally formulated with parenterally suitable diluents, dispersing, wetting, or suspending agents. These injectable preparations can be sterilized by filtration through a bacterial-retaining filter or formulated with sterilizing agents that dissolve or disperse in the injectable media.

Inhibition of DGAT-1 by the compounds of the present invention can be delayed by using a liquid suspension of crystalline or amorphous material with pool water solubility. The rate of absorption of the compounds depends upon their rate of dissolution, which, in turn, depends on their crystallinity. Delayed absorption of a parenterally administered compound can be accomplished by dissolving or suspending the compound in oil. Injectable depot forms of the compounds can also be prepared by microencapsulating the same in biodegradable polymers. Depending upon the ratio of compound to polymer and the nature of the polymer employed, the rate of release can be controlled. Depot injectable formulations are also prepared by entrapping the compounds in liposomes or microemulsions that are compatible with body tissues.

Solid dosage forms for oral administration of the present compounds include capsules, tablets, pills, powders, and granules. In such forms, the compound is mixed with at least one inert, therapeutically suitable excipient such as a carrier, fillet, extender, disintegrating agent, solution retarding agent, wetting agent, absorbent, or lubricant. With capsules, tablets, and pills, the excipient can also contain buffering agents. Suppositories for rectal administration can be prepared by mixing the compounds with a suitable non-irritating excipient that is solid at ordinary temperature but fluid in the rectum.

The present compounds can be micro-encapsulated with one or more of the excipients discussed previously. The solid dosage forms of tablets, dragees, capsules, pills, and granules can be prepared with coatings and shells such as enteric and release-controlling. In these forms, the compounds can be mixed with at least one inert diluent and can optionally include tableting lubricants and aids. Capsules can also optionally contain opacifying agents that delay release of the compounds in a desired part of the intestinal tract.

Transdermal patches have the added advantage of providing controlled delivery of the present compounds to the body. Such dosage forms are prepared by dissolving or dispensing the compounds in the proper medium. Absorption enhancers can also be used to increase the flux of the compounds across the skin, and the rate of absorption can be controlled by providing a rate controlling membrane or by dispersing the compounds in a polymer matrix or gel.

The compounds of the invention can be used in the form of pharmaceutically acceptable salts, esters, or amides derived from inorganic or organic acids. The term “pharmaceutically acceptable salts, esters and amides,” as used herein, include salts, zwitterions, esters and amides of compounds of disclosed herein which are, within the scope of sound medical judgment, suitable for use in contact with the tissues of humans and lower animals without undue toxicity, irritation, allergic response, and the like, are commensurate with a reasonable benefit/risk ratio, and are effective for their intended use.

Pharmaceutically acceptable salts are well-known in the art. The salts can be prepared during the final isolation and purification of the compounds or separately by reacting an amino group of the compounds with a suitable acid. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, malate, maleate, methanesulfonate, naphtlylenesulfonate, nicotinate, oxalate, pamoate, pectinate, persulfate, 3-phenylpropionate, picrate, oxalate, pivalate, propionate, succinate, tartrate, trichloroacetic, trifluoroacetic, glutamate, para-toluenesulfonate, undecanoate, hydrochloric, hydrobromic, sulfuric, phosphoric, and the like. The amino groups of the compounds can also be quaternized with alkyl chlorides, bromides, and iodides such as methyl, ethyl, propyl, isopropyl, butyl, lauryl, myristyl, stearyl, and the like.

Basic addition salts can be prepared during the final isolation and purification of the present compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine Quaternary amine salts derived from methylamine, dimethylamine, trimethylamine, triethylamine, diethylamine, ethylamine, tributlyamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine, ethylenediamine, ethanolamine, diethanolamine, piperidine, piperazine, and the like, are contemplated as being within the scope of the present invention.

Disorders that can be treated or prevented in a patient by administering to the patient, a therapeutically effective amount of compound of the present invention in such an amount and for such time as is necessary to achieve the desired result. The term “therapeutically effective amount,” refers to a sufficient amount of a compound of the invention to effectively ameliorate disorders by inhibiting DGAT-1 at a reasonable benefit/risk ratio applicable to any medical treatment. The specific therapeutically effective dose level for any particular patient depends upon a variety of factors including the disorder being treated and the severity of the disorder; the activity of the compound employed; the specific composition employed; the age, body weight, general health, sex, and diet of the patient; the time of administration, route of administration, rate of excretion; the duration of the treatment; and drugs used in combination or coincidental therapy.

The total daily dose of the compounds of the present invention necessary to inhibit the action of DGAT-1 in single or divided doses can be in amounts, for example, from about 0.01 to 50 mg/kg body weight. In a more preferred range, compounds of the present invention inhibit the action of DGAT-1 in a single or divided doses from about 0.05 to 25 mg/kg body weight. Single dose compositions can contain such amounts or submultiple doses thereof of the compounds of the present invention to make up the daily dose. In general, treatment regimens include administration to a patient in need of such treatment from about 1 mg to about 1000 mg of the compounds per day in single or multiple doses.

The compounds identified by the methods described herein can be administered as the sole pharmaceutical agent or in combination with one or more other pharmaceutical agents where the combination causes no unacceptable adverse effects. For example, the compounds of this invention can be combined with anti-obesity, or with known antidiabetic or other indication agents, and the like. Thus, the present invention also includes pharmaceutical compositions which include a therapeutically effective amount of a compound identified by the methods described herein, or a pharmaceutically acceptable salt thereof, a pharmaceutically acceptable carrier; and one of more pharmaceutical agents as disclosed hereinabove.

The present invention is described below in connection with certain preferred embodiments which are not intended to limit its scope. On the contrary, the present invention covers all alternatives, modifications, and equivalents as can be included within the scope of the claims. Routine experimentation, including appropriate manipulation of the reaction conditions, reagents used and sequence of the synthetic route, protection of any chemical functionality that can not be compatible with the reaction conditions, and deprotection at suitable point in the reaction sequence of the method are included in the scope of the invention. Suitable protecting groups and the methods for protecting and deprotecting different substituents using such suitable protecting groups are well know to those skilled in the art; examples of which can be found in T. Greene and P. Wuts, Protecting Groups in Chemical Synthesis (3^{rd} ed.), John Wiley & Sons, NY (1999), which is incorporated herein by reference in its entirety. Synthesis of the compounds of formula (I) can be accomplished by methods analogous to those described above and in the following examples. The following examples, which include preferred embodiments, illustrate the preferred practice of the present invention, it being understood that the examples are for the purpose of illustration of certain preferred embodiments and are presented to provide what is believed to be the most useful and readily understood description of its procedures and conceptual aspects. Finally, the compounds of the invention were named by ACD/ChemSketch version 5.06 (developed by Advanced Chemistry Development, Inc., Toronto, ON, Canada) or were given names consistent with ACD nomenclature.

### EXAMPLES

### Example 1

### N-(3-chlorophenyl)-N′-(4′-{(R)-hydroxy[(1R,2R)-2-(hydroxymethyl)cyclopentyl]methyl}-1,1′-biphenyl-4-yl)urea and N-(3-chlorophenyl)-N′-(4′-{(S)-hydroxy[(1R,2R)-2-(hydroxymethyl)cyclopentyl]methyl}-1,1′-biphenyl-4-yl)urea

### Example 1A

### cis-cyclopentane-1,2-dicarboxylic acid

To a solution containing ethyl 2-oxocyclohexanecarboxylate (100 g, 0.588 mol) in 300 mL of chloroform at 0° C. bromine (94 g, 0.588 mol) was added. The mixture was stirred overnight, washed with a saturated sodium bicarbonate solution and brine, dried over anhydrous sodium sulfate, filtered and concentrated under reduced pressure to remove the solvent. The residue was added drop wise to an ice-cold solution containing potassium hydroxide (140 g, 2.50 mol) in 800 mL of water. The reaction mixture was stirred for 2 hours and was extracted with diethyl ether. The aqueous phase was acidified with a 4.0 M hydrochloric acid solution and extracted with diethyl ether. The combined ethereal solution was dried over anhydrous sodium sulfate, filtered and evaporated under reduced pressure to give a yellow oil, which crystallized on standing to provide to Example 1A as colorless crystals

### Example 1B

### cyclopentane-1,2-dicarboxylic anhydride

A solution containing Example 1A (56.6 g, 0.358 mol) in 1500 mL of acetic anhydride was heated at reflux for 20 hours. The excess acetic anhydride was removed by distillation under reduced pressure. The oily residue was distilled to give Example 1B as a colorless oil. ¹H NMR (500 MHz, DMSO-d₆) δ ppm 1.07-2.02 (m, 6H) and 3.47-3.55 (m, 2H).

### Example 1C

### cis-2-(methoxycarbonyl)cyclopentanecarboxylic acid

Example 1B (40.2 g, 286.9 mmol) was dissolved in methanol (250 mL), and the mixture was then heated at 50-55° C. under N₂ for 16 hours. The reaction was concentrated under reduced pressure, and the residue was dried in vacuo to afford the desired product as a colorless oil. ¹H NMR (500 MHz, DMSO-d₆) δ ppm 1.54-1.61 (m, 1H), 1.66-1.76 (m, 1H), 1.80-1.91 (m, 4H), 3.95-3.02 (m, 2H), 3.54 (s, 3H), 12.05 (br s, 1H).

### Example 1D

### methyl cis-2-(4-bromobenzoyl)cyclopentanecarboxylate

Step A:

A solution of Example 1C (30.7 g, 178.3 mmol), SOCl₂ (39 mL, 535 mmol), and N,N-dimethylformamide (0.33 mL) in CH₂Cl₂ (383 mL) was stirred at room temperature overnight under N₂. The solvent was removed by rotary evaporation at <40° C., and the residue was dried in vacuo for 1 hour

Step B:

The intermediate described in step A was dissolved in bromobenzene (112.5 mL, 1.067 mol), and AlCl₃ (47.5 g, 357 mmol) was then added portion wise at <5° C. The reaction mixture turned dark brown, and was stirred at <5° C. for 4 hour under N₂. ¹H NMR showed that little starting material remained. The reaction mixture was then slowly poured into 700 mL ice-water, and then 350 mL ethyl acetate was added. After the mixture was stirred for 10 minutes, the aqueous (top) layer was separated, and extracted with 200 mL ethyl acetate. The combined organic layers were washed with water (2×350 mL) and saturated NaHCO₃ solution (70 mL), dried over Na₂SO₄, and filtered. Removal of solvent and drying in vacuo provided the desired product, which was used directly in the next step. ¹H NMR (500 MHz, DMSO-d₆) δ ppm 1.58-1.83 (m, 3H), 1.91-2.05 (m, 3H), 3.15-3.21 (m, 1H), 3.37 (s, 3H), 4.13-4.19 (m, 1H), 7.73 (d, J=8.59 Hz, 2H), 7.87 (d, J=8.90 Hz, 2H).

### Example 1E

### trans-2-(4-bromobenzoyl)cyclopentanecarboxylic acid

A solution of NaOH (42.9 g, 1.07 mol) in 234 mL water was added to a solution of Example 1D (178.3 mmol) in methanol (234 mL). The reaction mixture was stirred at room temperature overnight. ¹H NMR showed that little starting material remained. After 350 mL solvent was removed by rotary evaporation, the mixture was diluted with 350 mL water. The aqueous was acidified to pH 6 with concentrated HCl while keeping the internal temperature at <15° C. A precipitate formed, and stirring was continued for 1 hour. The solid precipitate was filtered, and rinsed with water. The dried filter cake was dissolved in 350 mL ethyl acetate, dried over Na₂SO₄, and filtered. Removal of solvent and drying in vacuo afforded the title compound. ¹H NMR (500 MHz, DMSO-d₆) δ ppm 1.55-1.85 (m, 4H), 1.93-2.03 (m, 1H), 2.09-2.15 (m, 1H), 3.15-3.21 (m, 1H), 4.00-4.06 (m, 1H), 7.75 (d, J=8.59 Hz, 2H), 7.93 (d, J=8.59 Hz, 2H), 12.21 (br s, 1H).

### Example 1F

### (1R,2R)-2-(4-bromobenzoyl)cyclopentanecarboxylic acid

A mixture of Example 1E (27.4 g, 92.2 mmol) and (R)-(+)-alpha-methyl-benzylamine (5.59 g, 46.1 mmol) in CH₃CN (268 mL) was heated to 90-95° C. under N₂ to provide a solution. The hot solution was allowed to cool slowly with slow stirring overnight. The crystallized solid was filtered, and rinsed with CH₃CN (12 mL). The filter cake was dried in vacuo to a constant weight. The solid was then dissolved in a hot (95° C.) mixture of solvent (62 mL ethanol and 124 mL water) under N₂. The hot solution was allowed to cool slowly with slow stirring overnight. The solid was filtered, rinsed with 15 mL of 1:2 ethanol/water, and dried in vacuo to a constant weight. The white solid was stirred with 1N HCl (120 mL) and ethyl acetate (120 mL) for 10 minutes. The organic layer was separated, washed with water (2×48 mL), and dried over Na₂SO₄. Removal of solvent and drying in vacuo provided an off-white solid (>94% ee based on chiral HPLC). Chiral HPLC method: Chiracel OJ analytical column, 2:98 ethanol/hexanes (both containing 0.1% trifluoroacetic acid), 1.5 mL/min flow rate, retention times were 18.90 min and 22.18 min for the (S,S) and (R,R) isomers, respectively ¹H NMR (500 MHz, DMSO-d₆) δ ppm 1.55-1.85 (m, 4H), 1.93-2.03 (m, 1H), 2.09-2.15 (m, 1H), 3.15-3.21 (m, 1H), 4.00-4.06 (m, 1H), 7.75 (d, J=8.59 Hz, 2H), 7.93 (d, J=8.59 Hz, 2H), 12.21 (br s, 1H).

### Example 1G

### methyl (1R,2R)-2-(4-bromobenzoyl)cyclopentanecarboxylate

A suspension of Example 1F (7.96 g, 26.8 mmol), iodomethane (2.5 mL, 40.2 mmol), and NaHCO₃ (6.75 g, 80.4 mmol) in N,N-dimethylformamide (94 mL) was stilled at room temperature under N₂ for 16 hours. ¹H NMR showed that little starting material remained. Water (262 mL) was added to the reaction mixture, and the organics were removed under reduced pressure. The aqueous was acidified to pH <7 by the addition of 1 N HCl, while keeping the internal temperature of the <15° C. Ethyl acetate (300 mL) was added, and the layers were separated. The aqueous layer was extracted with ethyl acetate (2× 200 mL). The combined organic layers were washed with brine, dried over Na₂SO₄ and concentrated. The residue was purified by chromatography on SiO₂ gel, eluting with 0-5% ethyl acetate in hexanes, to provide the title compound. ¹H NMR (500 MHz, DMSO-d₆) δ ppm 1.59-1.84 (m, 4H), 1.97-2.04 (m, 1H), 2.11-2.19 (m, 1H), 3.24-3.29 (m, 1H), 3.56 (s, 3H), 4.02-4.08 (m, 1H), 7.76 (d, J=8.90 Hz, 2H), 7.93 (d, J=8.59 Hz, 2H).

### Example 1H

### methyl (1R,2R)-2-[(4′-nitro-1,1′-biphenyl-4-yl)carbonyl]cyclopentanecarboxylate

To an ambient slurry of Example 1G (2 g, 6.4 mmol), 4-nitrophenyl boronic acid (2.1 g, 12.8 mmol) and KF, (1.12 g, 19.3 mmol) in dimethoxyethane/toluene/ethanol/H₂O (10/1/6/3 ratio, 30 mL) palladium tetrakis(triphenylphosphine) (75 mg, 0.06 mmol) in a single portion was added. The reaction was heated to 90° C. overnight, cooled to room temperature, filtered through celite, washed with ethyl acetate, concentrated and purified on a flash column, eluting with 0-15% ethyl acetate in hexanes, to provide the title compound

¹H NMR (500 MHz, DMSO-d₆) δ ppm 1.58-1.89 (m, 4H), 1.99-2.07 (m, 1H), 2.16-2.23 (m, 1H), 3.24-3.29 (m, 1H), 3.58 (s, 3H), 4.11-4.18 (m, 1H), 7.96 (d, J=8.59 Hz, 2H), 8.06 (d, J=8.90 Hz, 2H), 8.14 (d, J=8.59 Hz, 2H), 8.34 (d, J=8.90 Hz, 2H); MS (ESI) m/z 354.0 [M+H]⁺

### Example 1I

### methyl (1R,2R)-2-[(4′-amino-1,1′-biphenyl-4-yl)carbonyl]cyclopentanecarboxylate

A mixture of Example 1I (2.206 g, 6.24 mmol), iron powder (1.046 g, 18.7 mmol), and NH₄Cl (334 mg, 6.24 mmol) in a mixture of solvents (90 mL of ethanol and 25 mL of water) was heated to 85° C. under N₂ for 2 hours. The reaction mixture was filtered through celite, treated with aqueous saturated sodium bicarbonate (50 mL) and extracted with ethyl acetate. The organic layer was washed with brine, dried over Na₂SO₄, filtered and concentrated. The residue was concentrated to provide the title compound without further purification. ¹H NMR (500 MHz, DMSO-d₆) δ ppm 1.54-1.86 (m, 4H), 1.98-2.07 (m, 1H), 2.13-2.22 (m, 1H), 3.25-3.29 (m, 1H), 3.57 (s, 3H), 4.04-4.10 (m, 1H), 5.41 (s, 2H), 6.86(d, J=8.90 Hz, 2H), 7.48 (d, J=8.59 Hz, 2H), 7.70 (d, J=8.90 Hz, 2H), 7.98 (d, J=8.60 Hz, 2H); MS (ESI) m/z 324.0 [M+H]⁺.

### Example 1J

### methyl (1R,2R)-2-{[4′-({[(3-chlorophenylaminocarbonyl}amino)-1,1′-biphenyl-4-yl]carbonyl}cyclopentanecarboxylate

A scintillation vial was charged with Example 1I (20 mg, 0.06 mmol), 1-chloro-3-isocyanatobenzene (9 mg, 0.07 mmol) and tetrahydrofuran (6 mL). It was placed in a shaker at room temperature overnight. The mixture was concentrated and purified by RP-HPLC (preparative reversed-phase chromatography was performed using a Zorbax SB-C18 7 μM 21.2×250 mm column with UV detection analyzed at 220 and 254 nM. Preparative method; (water with 0.1% trifluoroacetic acid and CH₃CN with 0.1% trifluoroacetic acid gradient) 5-95% CH₃CN over 30 minutes at 15 mL/min.) to provide the title product. ¹H NMR (500 MHz, DMSO-d₆) δ ppm 1.56-1.87 (m, 4H), 2.00-2.07 (m, 1H), 2.16-2.23 (m, 1H), 3.30-3.34 (m, 1H), 3.58 (s, 3H), 4.09-4.14 (m, 1H), 7.02-7.05 (m, 1H), 7.28-7.33 (m, 2H), 7.60 (d, J=8.84 Hz, 2H), 7.72-7.74 (m, 3H), 7.82 (d, J=8.55 Hz, 2H), 8.06 (d, J=8.24 Hz, 2H), 8.96 (d, J=2.44 Hz, 2H); MS (ESI) m/z 477 [M+H]⁺

### Example 1K

### N-(3-chlorophenyl)-N′-{(R)-hydroxy[(1R,2R)-2-(hydroxymethyl)cyclopentyl]methyl}-1,1′-biphenyl-4-yl)urea and N-(3-chlorophenyl)-N′-(4′-{(S)-hydroxy[(1R,2R)-2-(hydroxymethyl)cyclopentyl]methyl}-1,1′-biphenyl-4-yl)urea

A 25 mL round-bottom flask was charged with Example 1J (48 mg, 0.1 mmol) and tetrahydrofuran (8 mL). After cooling to 0° C., a solution of lithium aluminum hydride in tetrahydrofuran (0.4 mL of 1.0 M, 0.4 mmol) was added to the reaction flask. The cooling bath was removed, and the reaction stirred at room temperature for 2 hours. The reaction was quenched with saturated ammonium chloride solution (10 mL), and the aqueous layer was extracted with ethyl acetate (50 mL). The combined organic layer was dried over Na₂SO₄, concentrated, and purified by RP-HPLC to provide two diastereomers. Retention times of the two diastereomers were determined by RP-HPLC (Agilent ZORBAX SB-C18 column 5 um, 4.6×250 mm, solvent flow: 1.5 ml/min, stop time: 20 minutes, beginning with 100% H₂O (with 0.1% trifluoroacetic acid) for 0-1 min., 1-15 min: 0% CH₃CN to 100% CH₃CN, 15-18 min: 100% CH₃CN, 18-19 minutes: 100% to 0% CH₃CN, 19-20 minutes: 100% H₂O (with 0.1% trifluoroacetic acid). Diastereomer with retention time=12.775 minutes had ¹H NMR (500 MHz, DMSO-d₆) δ ppm 1.35-1.52 (m, 4H), 1.55-1.67 (m, 2H), 1.81-1.89 (m, 2H), 3.08-3.16 (m, 2H), 4.37 (t, J=4.91 Hz, 1H), 4.50 (t, J=4.91 Hz, 1H), 5.13 (d, J=4.60 Hz, 1H), 7.01-7.03 (m, 1H), 7.30 (d, J=7.67 Hz, 2H), 7.37 (d, J=8.29 Hz, 2H), 7.53-7.62 (m, 6H), 7.72-7.73 (m, 1H), 8.84 (s, 1H), 8.90 (s, 1H); MS (ESI) m/z 449.1 [M−H]⁺; diastereomer with retention time of 13.082 minutes had ¹H NMR (500 MHz, DMSO-d₆) δ ppm 1.16-1.51 (m, 5H), 1.63-1.71 (m, 1H), 1.79-1.86 (m, 1H), 1.99-2.07 (m, 1H), 3.26-3.35 (m, 2H), 4.32 (d, J=8.28 Hz, 1H), 4.79 (br, s, 1H), 5.48 (br s, 1H), 7.01-7.03 (m, 1H), 7.30 (d, J=7.67 Hz, 2H), 7.37 (d, J=8.29 Hz, 2H), 7.52-7.62 (m, 6H), 7.72-7.73 (m, 1H), 8.83 (s, 1H), 8.89 (s, 1H); MS (ESI) m/z 449.1 [M−H]⁺.

### Example 2

### N-(3-chlorophenyl)-N′-(4′-{[(1R,2R)-2-(1-hydroxy-1-methylethyl)cyclopentyl]carbonyl}-1,1′-biphenyl-4-yl)urea

### Example 2A

### (4-bromophenyl)[(1R,2R)-2-(1-hydroxy-1-methylethyl)cyclopentyl]methanone

Step A

Sodium borohydride (0.25 g, 6.59 mmol) was added in portions to a solution of Example 1G (1.95 g, 6.27 mmol) in tetrahydrofuran (20 mL) and methanol (5 mL) maintained at 0° C. The reaction was allowed to warm to rt over 30 minutes and then stirred at rt for 1 hour. The reaction was quenched by addition of water (50 mL) and extracted with ethyl acetate. The organic extracts were washed with water, brine, dried (MgSO₄), filtered and concentrated to a brown oil, which was used in the next step.

Step B

Methyl magnesium bromide (11 mL, 3M solution in diethylether) was added drop wise to a solution of the crude product obtained from step A in tetrahydrofuran (40 mL) maintained at 0° C. The reaction was allowed to warm up to room temperature overnight and then quenched by careful addition of water and aqueous dilute HCl. The mixture was extracted with ethyl acetate, and the organic layers were washed with water; brine, dried (MgSO₄), filtered and concentrated to a clear oil, which was used as is in the next step.

Step C

The product from step B was placed in dichloromethane (30 mL) with silica gel (1 g), and pyridinium chlorochromate (2.02 g, 9.4 mmol) was added at rt. The reaction was stirred at rt for 24 h and was then filtered through a pad of silica gel. The filtrate was concentrated and purified by flash chromatography, using 10% ethyl acetate/hexanes as the eluent, to afford the title compound. ¹H NMR (300 MHz, DMSO-d₆) δ ppm 0.94 (s, 3H), 1.06 (s, 3H), 1.42-1.77 (m, 5H), 1.93-2.02 (m, 1H), 2.53-2.61 (m, 1H), 3.77-3.85 (m, 1H), 4.16 (broad s, 1H), 7.74 (d, J=8.5 Hz, 2H), 7.94 (d, J=8.5 Hz, 2H).

### Example 2B

### [(1R,2R)-2-(1-hydroxy-1-methylethyl)cyclopentyl](4′-nitro-1,1′-biphenyl-4-yl)methanone

4-Nitro phenyl boronic acid (0.69 g, 4.1 mmol) was added to a suspension of Example 2A (0.92 g, 2.94 mmol), potassium fluoride (0.51 g, 8.8 mmol), and tetrakis(triphenylphosphine)palladium(0) (0.28 g, 0.24 mmol) in a solvent mixture (10:1:6:3:DME:PhCH₃:ethanol, H₂O, 40 mL) and degassed with nitrogen for 10 minutes. The reaction mixture was then heated to 90° C. for 15 hours, cooled, quenched with water (40 mL), and extracted with ethyl acetate. The organic extracts were washed with water and brine, dried (MgSO₄), concentrated and purified by flash chromatography, using 30% ethyl acetate/hexanes as eluent, to provide the title compound. ¹H NMR (400 MHz, DMSO-d₆) δ ppm 0.96 (s, 3H), 1.08 (s, 3H), 1.50-1.58 (m, 2H), 1.62-1.67 (m, 2H), 1.73-1.79 (m, 1H), 1.99-2.07 (m, 1H), 2.61-2.66 (q, J=7.7 Hz, 1H), 3.89-3.92 (m, 1H), 4.17 (s, 1H), 7.95 (d, J=8.6 Hz, 2H), 8.04 (d, J=8.9 Hz, 2H), 8.14 (d, J=8.6 Hz, 2H), 8.35 (d, J=8.9 Hz, 2H); MS (ESI) m/z 352.1 [M−H]⁻.

### Example 2C

### N-(3-chlorophenyl)-N′-(4′-{[(1R,2R)-2-(1-hydroxy-1-methylethyl)cyclopentyl]carbonyl}-1,1′-biphenyl-4-yl)urea

Step A

Example 2B (0.6 g, 1.7 mmol) was placed along with iron (0.19 g, 3.4 mmol) and ammonium chloride (0.1 g, 1.87 mmol) in ethanol (10 mL) and water (4 mL) and heated at 90° C. for 2 hours. The reaction mixture was then cooled, filtered over wet celite, and the filtrate diluted with water. The resultant precipitate was filtered, and the filtrate extracted with ethyl acetate. The organic extracts were washed with water and brine, dried (MgSO₄), filtered, concentrated and combined with the solid obtained from filtration and carried forward to the next step without further purification.

Step B

The crude product from step A (0.4 g, 1.24 mmol) was treated with 3-chlorophenyl isocyanate (0.18 mL, 1.48 mmol) in tetrahydrofuran (20 mL) and stirred at room temperature for 2 days. The reaction mixture was quenched with water and extracted with ethyl acetate. The organic extracts were washed with water and brine, dried (MgSO₄), filtered and concentrated to a yellow solid. The crude solid was taken up in methanol, and the resultant slurry filtered to afford the title compound. The filtrate was purified by RP-HPLC (Preparative reversed-phase chromatography was performed using a Zorbax SB-C18 7μM 21.2×250 mm column with UV detection analyzed at 220 and 254 nM. Preparative method: (Water with 0.1% trifluoroacetic acid and CH₃CN with 0.1% trifluoroacetic acid gradient) 5-95% CH₃CN over 30 minutes at 15 mL/min.) to afford an additional crop of the title compound. ¹H NMR (400 MHz, DMSO-d₆) δ ppm 0.96 (s, 3H), 1.08 (s, 3H), 1.49-1.55 (m, 2H), 1.62-1.67 (m, 2H), 1.73-1.79 (m, 1H), 1.99-2.07 (m, 1H), 2.63 (q, J=7.9 Hz, 1H), 3.89-3.92 (m, 1H), 4.15 (s, 1H), 7.03 (dt, J=2.1, 6.7, 1H), 7.29-7.34 (m, 2H), 7.59 (d, J=8.6 Hz, 2H), 7.71 (d, J=8.9 Hz, 2H), 7.81 (d, J=8.6 Hz, 2H), 8.06 (d, J=8.9 Hz, 2H), 8.93 (s, 2H); MS (ESI) m/z 459.2 [M−17]⁻

### Example 3

### N-(4′-{[(1R,2R)-2-(1-hydroxy-1-methylethyl)cyclopentyl]carbonyl}-1,1′-biphenyl-4-yl)-N′-phenylurea

Example 3 was prepared using the procedure described for the synthesis of the intermediate of step B of Example 2C, substituting phenyl isocyanate for 3-chloro phenyl isocyanate. ¹H NMR (400 MHz, DMSO-d₆) δ ppm 0.96 (s, 3H), 1.08 (s, 3H), 1.46-1.56 (m, 2H), 1.59-1.69 (m, 2H), 1.72-1.80 (m, 1H), 1.99-2.07 (m, 1H), 2.61 (q, J=7.9 Hz, 1H), 3.86-3.92 (m, 1H), 4.17 (s, 1H), 6.98 (t, J=7.3, 1H), 7.28 (t, J=7.6 Hz, 2H) 7.47 (d, J=7.6 Hz, 2H), 7.60 (d, J=8.8 Hz, 2H), 7.70 (d, J=8.8 Hz, 2H), 7.80 (d, J=8.5 Hz, 2H), 8.06 (d, J=8.5 Hz, 2H), 8.77 (s, 1H), 8.91 (s, 1H); MS (ESI) m/z 441.2 [M−H]⁻.

### Example 4

### N-(4′-{(S)-hydroxy[(1R,2R)-2-(hydroxymethyl)cyclopentyl]methyl}-1,1′-biphenyl-4-yl)-N′-phenylurea and N-(4′-{(R)-hydroxy[(1R,2R)-2-(hydroxymethyl)cyclopentyl]methyl}-1,1′-biphenyl-4-yl)-N′-phenylurea

Step A

A solution of Example 14A (44 mg, 0.1 mmol) in tetrahydrofuran (0.4 mL) and methanol (0.1 mL) at ambient temperature was treated with sodium borohydride (5 mg, 0.13 mmol). The homogeneous yellow solution turned colorless in ten minutes. After one hour, the reaction was quenched by slow addition of distilled water, stirred five minutes, then 1M H₂SO₄ added and stirred an additional five minutes. The reaction mixture was then diluted with ethyl acetate, and the aqueous layer basified to pH 10 with 1M K₂CO₃. The layers were separated, and the organic extract was washed with brine, dried (Na₂SO₄), filtered and concentrated to give 40 mg of a yellow solid.

Step B

One half of this yellow solid from Step A (20 mg) was purified by flash silica gel chromatography using a gradient elution of methanol in dichloromethane to afford two diastereomers. The higher R_{f} diastereomer isolated as an off-white solid had ¹H NMR (300 MHz, DMSO-d₆) δ ppm 1.11-1.52 (m, 5 H), 1.59-1.72 (m, 1 H), 1.83 (s, J=6.78 Hz, 1 H), 1.95-2.11 (m, 1 H), 3.2-3.3 (m, 2H), 4.32 (dd, J=7.97, 3.56 Hz, 1 H), 4.79 (t, J=4.75 Hz, 1 H), 5.48 (d, J=3.39 Hz, 1 H), 6.97 (d, J=7.46 Hz, 1 H), 7.24-7.32 (m, 2 H), 7.36 (d, J=8.48 Hz, 2 H), 7.47 (d, J=7.46 Hz, 2 H), 7.50-7.64 (m, 6 H), 8.72 (s, 1 H), 8.77-8.82 (s, 1 H), MS (ESI, methanol/NH₄OH) m/z 416 [M]⁺, 415 [M−H]⁻. Lower R_{f} diastereomer was isolated as an off-white solid with ¹H NMR (300 MHz, DMSO-d₆) δ ppm 1.29-1.53 (m, 4 H) 1.53-1.69 (m, 2 H) 1.74-1.93 (m, 2 H) 3.03-3.19 (m, 2 H) 4.38 (t, J=5.09 Hz, 1 H) 4.4-4.54 (m, 1 H) 5.13 (d, J=4.41 Hz, 1 H) 6.97 (t, J=7.29 Hz, 1 H) 7.24-7.32 (m, 2 H) 7.37 (d, J=8.14 Hz, 2 H) 7.46 (d, J=7.46 Hz, 2 H) 7.51-7.65 (m, 6 H) 8.69 (s, 1 H) 8.76 (s, 1 H); MS (ESI, methanol/NH₄OH) m/z 417 [M+H]⁺, 415 [M−H]⁻.

### Example 5

### N-4′-{[(1R,2R)-2-(hydroxymethyl)cyclopentyl]methyl}-1,1′-biphenyl-4-yl)-N′-phenylurea

A solution of the product from Step A of Example 4 (20 mg, 0.045 mmol) in tetrahydrofuran (0.4 mL) and methanol (0.1 mL) was charged with 20% Pd(OH)₂ on carbon (10 mg). The reaction was swept with nitrogen and stirred under a hydrogen atmosphere (balloon) for 2.3 hours at ambient temperature. Analysis by LC/MS revealed remaining starting material. Therefore, the flask was swept with nitrogen, more Pd(OH)₂ on carbon (10 mg) added, swept with nitrogen and stirred under a hydrogen atmosphere for an additional fourteen hours. The reaction was swept with nitrogen for five minutes, the black heterogeneous mixture was filtered through a plug of silica (2 g), rinsed with 95/5 dichloromethane/methanol and concentrated to give a white solid. Purification by flash silica gel chromatography using gradient elution of methanol in dichloromethane gave the title compound as a white solid ¹H NMR (300 MHz, DMSO-d₆) δ ppm 1.15-1.62 (m, 6 H), 1.63-1.74 (m, 2 H), 1.77-1.89 (m, 1 H), 2.80 (dd, J=13.39, 5.59 H), 1H), 3.13-3.28 (m, 2 H), 4.42 (t, J=5.26 Hz, 1 H), 6.97 (t, J=7.29 Hz, 1 H), 7.20-7.33 (m, 4 H), 7.46 (d, J=7.46 Hz, 2 H), 7.50-7.63 (m, 6 H), 8.71 (s, 1 H), 8.77 (s, 1 H); MS (ESI, methanol/NH₄OH) m/z 401 [M+H]⁺, 423 [M+Na]+, 399 [M−H]⁻.

### Example 6

### methyl (1R,2R)-2-{4-[5-({[(3-chlorophenyl)amino]carbonyl}amino)thien-2-yl]benzoyl}cyclopentanecarboxylate

### Example 6A

### methyl (1R,2R)-2-[4-(4,4,5,5-tetramethyl-1,3,2-dioxaborolan-2-yl)benzoyl]cyclopentanecarboxylate

To a solution of Example 1G (1.9 g, 6.4 mmol), bis(pinacolato)diboron (1.6 g, 6.4 mmol) and N,N-dimethylformamide (35 mL) potassium acetate (1.9 g, 19 mmol) and palladium (II) acetate (430 mg, 1.9 mmol) were added. The reaction mixture was heated to 85° C. for 3 hours, then cooled to room temperature. The mixture was filtered through a plug of silica gel and rinsed with ethyl acetate. The filtrate was concentrated and further purified by flash chromatography (ethyl acetate/Hexane; 1/10) to give rise to pale yellow oil, ¹H NMR (500 MHz, DMSO-d₆) δ ppm 1.32 (s, 12H), 1.47-1.88 (m, 4H), 1.99-2.20 (m, 2H), 3.28 (m, 1H), 3.56 (s, 3H), 4.08 (m, 1H), 7.82 (d, J=8.29 Hz, 2H), 7.98 (d, J=8.29 Hz, 2H); MS (DCI/NH₃) m/z 359 [M+H]⁺.

### Example 6B

### methyl (1R,2R)-2-[4-(5-nitrothien-2-yl)benzoyl]cyclopentanecarboxylate

To an ambient slurry of 6A (270 mg, 0.75 mmol), 2-bromo-5-nitro-thiophene (156 mg, 0.75 mmol) and potassium fluoride (130 mg, 2.24 mmol) in dimethyoxyethane/toluene/ethanol/H₂O (10/1/6/3 ratio, 3 mL) was added palladium tetrakis(triphenylphosphine) (10 mg, 0.0086 mmol) in a single portion. The reaction was heated at 90° C. overnight, cooled to room temperature, filtered through celite, washed with ethyl acetate, concentrated and purified by flash chromatography on SiO₂ column (0-5% ethyl acetate in hexanes) to provide the title compound as yellow solid. ¹H NMR (500 MHz, DMSO-d₆) δ ppm 1.54-1.87 (m, 4H), 1.99-2.20 (m, 2H), 3.30 (m, 1H), 3.57 (s, 3H), 4.12 (m, 1H), 7.84 (d, J=4.27 Hz, 1H), 8.01 (d, J=8.54 Hz, 2H), 8.10 (d, J=8.54 Hz, 2H), 8.23 (d, J=4.27 Hz, 1H); MS (DCI/NH₃) m/z 360 [M+H]⁺.

### Example 6C

### methyl (1R,2R)-2-[4-(5-aminothien-2-yl)benzoyl]cyclopentanecarboxylate

A mixture of 6B (200 mg, 0.56 mmol), iron powder (188 mg, 3.36 mmol), and NH₄Cl (30 mg, 0.56 mmol) in a mixture of solvents (8 mL of ethanol and 2 mL of water) was heated at 85° C. under N₂ for 1 hour. The reaction mixture was cooled to about room temperature, filtered through celite, washed with ethyl acetate, and concentrated. The reaction mixture was basified with saturated NaHCO₃ solution and extracted with ethyl acetate. The organic layer was washed with brine, dried over Na₂SO₄, and filtered. Removal of solvent provided the title compound. ¹H NMR (500 MHz, DMSO-d₆) δ ppm 1.52-1.86 (m, 4H), 1.96-2.21 (m, 2H), 3.27 (m, 1H), 3.56 (s, 3H), 4.03 (m, 1H), 5.93 (d, J=3.99 Hz, 1H), 6.13 (s, 2H), 7.25 (d, J=3.99 Hz, 1H), 7.48 (d, J=8.60 Hz, 2H), 7.88 (d, J=8.60 Hz, 2H); MS (DCI/NH₃) m/z 330 [M+H]⁺.

### Example 6D

### methyl (1R,2R)-2-{4-[5-({[(3-chlorophenyl)amino]carbonyl}amino)thien-2-yl[benzoyl}cyclopentanecarboxylate

A scintillation vial was charged with 6C (50 mg, 0.15 mmol), 3-chlorophenyl isocyanate (23 mg, 0.15 mmol) and tetrahydrofuran (3 mL). It was placed in a shaker at room temperature for 2 hours. The mixture was concentrated and purified by RP-HPLC (Preparative reversed-phase chromatography was performed using a Zorbax SB-C18 7 uM 21.2×250 mm column with UV detection analyzed at 220 and 254 nM. Preparative method: (water with 0.1% trifluoroacetic acid and CH₃CN with 0.1% trifluoroacetic acid gradient) 5-95% CH₃CN over 30 minutes at 15 mL/min.) to provide the title product ¹H NMR (500 MHz, DMSO-d₆) δ ppm 1.65-1.85 (m, 4H), 1.97-2.22 (m, 2H), 3.30 (m, 1H), 3.57 (s, 3H), 4.07 (m, 1H), 6.66 (d, J=3.97 Hz, 1H), 7.06 (m, 1H), 7.33 (m, 2H), 7.46 (d, J=3.97 Hz, 1H), 7.71 (d, J=8.85 Hz, 2H), 7.72 (s, 1H), 7.98 (d, J=8.85 Hz, 2H), 9.10 (s, 1H), 10.06 (s, 1H); MS (DCI/NH₃) m/z 483 [M+H]⁺.

### Example 7

### methyl (1R,2R)-2-(4-{5-[(anilinocarbonyl)amino]thien-2-yl}benzoyl)cyclopentanecarboxylate

Example 7 was prepared using the same procedure as described for Example 6D substituting phenyl isocyanate for 3-chlorophenyl isocyanate ¹H NMR (500 MHz, DMSO-d₆) δ ppm 1.65-1.86 (m, 4H), 1.98-2.22 (m, 2H), 3.30 (m, 1H), 3.57 (s, 3H), 4.06 (m, 1H), 6.62 (d, J=3.97 Hz, 1H), 7.01 (t, J=7.32 Hz, 1H), 7.31 (m, 2H), 7.46 (d, J=3.97 Hz, 1H), 7.48 (d, J=7 63 Hz, 2H), 7.70 (d, J=8.54 Hz, 2H), 7.98 (d, J=8.54 Hz, 2H), 8.88 (s, 1H), 9.95 (s, 1H); MS (DCI/NH₃) m/z 449 [M+H]⁺.

### Example 8

### methyl (1R,2R)-2-(4-{5-[({[3-(trifluoromethyl)phenyl]amino]carbonyl)amino]thien-2-yl}benzoyl)cyclopentanecarboxylate

Example 8 was prepared using the procedure as described for Example 6D, substituting 3-trifluromethyl-phenyl isocyanate for 3-chlorophenyl isocyanate ¹H NMR (500 MHz, DMSO-d₆) δ ppm 1.65-1.87 (m, 4H), 1.98-2.21 (m, 2H), 3.30 (m, 1H), 3.57 (s, 3H), 4.07 (m, 1H), 6.67 (d, J=3.97 Hz, 1H), 7.36 (d, J=7.93 Hz, 1H), 7.46 (d, J=3.97 Hz, 1H), 7.54 (t, J=7.93 Hz, 1H), 7.64 (d, J=8.85 Hz, 1H), 7.72 (d, J=8.54 Hz, 2H), 7.98 (d, J=8.54 Hz, 2H), 8.03 (s, 1H), 9.27 (s, 1H), 10.13 (s, 1H); MS (DCI/NH₃) m/z 517 [M+H]⁺.

### Example 9

### methyl (1R,2R)-2-{4-[5({[(3-chlorophenyl)amino]carbonyl}amino)pyridin-2-yl]benzoyl}cyclopentanecarboxylate

### Example 9A

### methyl (1R,2R)-2-[4-(5-aminopyridin-2-yl)benzoyl]cyclopentanecarboxylate

To an ambient slurry of Example 6A (0.100 g, 0.279 mmol), 3-amino-6-bromopyridine (0.048 g, 0.279 mmol) and KF (0.049 g, 0.837 mmol) in dimethoxyethane/toluene/ethanol/H₂O (10/1/6/3 ratio, 30 mL) was added palladium tetrakis(triphenylphosphine) (5 mg, 0.004 mmol) in a single portion. The reaction was heated at 90° C. overnight, cooled to room temperature, filtered through celite, washed with ethyl acetate, concentrated and purified by RP-HPLC (Preparative reversed-phase chromatography was performed using a Zorbax SB-C18 7 uM 21.2×250 mm column with UV detection analyzed at 220 and 254 nM. Preparative method: (water with 0.1% trifluoroacetic acid and CH₃CN with 0.1% trifluoroacetic acid gradient) 5-95% CH₃CN over 30 minutes at 15 ml/min.) to provide the title compound. MS (ESI) m/z 325.0 [M+H]⁺.

### Example 9B

### methyl (1R,2R)-2-{4-[5-({[(3-chlorophenyl)amino]carbonyl}amino)pyridin-2-yl]benzoyl}cyclopentanecarboxylate

A scintillation vial was charged with Example 9A (5 mg, 0.015 mmol), 3-chlorophenyl isocyanate (3 mg, 0.02 mmol) and tetrahydrofuran (6 mL). It was placed in a shaker at room temperature overnight. The mixture was concentrated and purified by RP-HPLC (Preparative reversed-phase chromatography was performed using a Zorbax SB-C18 7 μM 21.2×250 mm column with UV detection analyzed at 220 and 254 nM. Preparative method: (water with 0.1% trifluoroacetic acid and CH₃CN with 0.1% trifluoroacetic acid gradient) 5-95% CH₃CN over 30 minutes at 15 mL/min.) to provide the title product ¹H NMR (500 MHz, DMSO-d₆) δ ppm 1.56-1.86 (m, 4H), 2.00 (m, 1H), 2.21 (m, 1H), 3.30-3.34 (m, 1H), 3.58 (s, 3H), 4.09-4.14 (m, 1H), 7.02-7.05 (m, 1H), 7.28-7.33 (m, 2H), 7.40-7.44 (m, 1H), 7.57 (m, 1H), 7.69 (d, J=8.55 Hz, 2H), 8.09 (d, J=8.55 Hz, 2H), 8.21 (m, 1H), 8.97 (s, 1H), 10.16 (s, 1H), 10.21 (s, 1H); MS (ESI) m/z 478.1 [M+H]⁺.

### Example 10

### (1R,2R)-2-{4-[5-({[(3-chlorophenyl)amino]carbonyl}amino)thien-2-yl]benzoyl}cyclopentanecarboxylic acid

A scintillation vial was charged with Example 6D (30 mg, 0.06 mmol), lithium hydroxide monohydrate (10 mg, 0.24 mmol) and a mixed solvent (2 mL of tetrahydrofuran, 1 mL of H₂O). It was placed in a shaker at room temperature overnight. The mixture was acidified with 10% HCl, concentrated, and purified by RP-HPLC (Preparative reversed-phase chromatography was performed using a Zorbax SB-C18 7 uM 21.2×250 mm column with UV detection analyzed at 220 and 254 nM. Preparative method: (water with 0.1% trifluoroacetic acid and CH₃CN with 0.1% trifluoroacetic acid gradient) 5-95% CH₃CN over 30 minutes at 15 mL/min.) to provide the title product. ¹H NMR (500 MHz, DMSO-d₆) δ ppm 1.64-1.85 (m, 4H), 1.97-2.20 (m, 2H), 3.20 (m, 1H), 4.04 (m, 1H), 6.65 (d, J=3.97 Hz, 1H), 7.06 (m, 1H), 7.33 (m, 2H), 7.46 (d, J=3.97 Hz, 1H), 7.71 (d, J=8.54 Hz, 2H), 7.72 (s, 1H), 7.99 (d, J=8.54 Hz, 2H), 9.09 (s, 1H), 10.05 (s, 1H), 12.22 (s, 1H); MS DCI/NH₃) m/z 469 [M+H]⁺.

### Example 11

### (1R,2R)-2-(4-{5-[(anilinocarbonyl)amino]thien-2-yl}benzoyl)cyclopentanecarboxylic acid

Example 11 was prepared using the same procedure as described for Example 10 substituting Example 7 for Example 6D ²H NMR (500 MHz, DMSO-d₆) δ ppm 1.64-1.85 (m, 4H), 1.97-2.20 (m, 2H), 3.21 (m, 1H), 4.03 (m, 1H), 6.62 (d, J=3.97 Hz, 1H), 7.01 (m, 1H), 7.31 (m, 2H), 7.45 (d, J=3.97 Hz, 1H), 7.48 (d, J=8.55 Hz, 2H), 7.70 (d, J=8.55 Hz, 2H), 7.79 (d, J=8.55 Hz, 2H), 8.87 (s, 1H), 9.94 (s, 1H), 12.23 (s, 1H); MS (DCI/NH₃) m/z 435 [M+H]⁺.

### Example 12

### (1R,2R)-2-(4-{5-[({[3-(trifluoromethyl)phenyl]amino}carbonyl)amino]thien-2-yl}benzoyl)cyclopentanecarboxylic acid

Example 12 was prepared using the same procedure as described for Example 10, substituting Example 8 for Example 6D. ¹H NMR (500 MHz, DMSO-d₆) δ ppm 1.64-1.85 (m, 4H), 1.97-2.20 (m, 2H), 3.20 (m, 1H), 4.04 (m, 1H), 6.67 (d, J=3.97 Hz, 1H), 7.36 (d, J=7.63 Hz, 1H), 7.46 (d, J=3.97 Hz, 1H), 7.54 (d, J₁=8.23 Hz, J₂=7.63 Hz, 1H), 7.63 (d, J=8.23 Hz, 1H), 7.72 (d, J=8.54 Hz, 2H), 7.99 (d, J=8.54 Hz, 2H), 8.03 (s, 1H), 9.26 (s, 1H), 10.12 (s, 1H), 12.22 (s, 1H); MS (DCI/NH₃) m/z 503 [M+H]⁺.

### Example 13

### (1R,2R)-2-[(4′-{]anilino(cyanoimino)methyl]amino}-1,1′-biphenyl-4-yl)carbonyl]cyclopentanecarboxylic acid

### Example 13A

### phenyl N′-cyano-N-phenylimidocarbamate

Aniline (500 μL, 5.48 mmol) was combined with diphenyl N-cyanocarbonimidate (1.31 g, 5.48 mmol) and acetonitrile (20 mL) at room temperature. After 18 hours the resulting white powder was collected by filtration. ¹H NMR (300 MHz, DMSO-d₆) δ ppm 7.21-7.33 (m, 4 H) 7.37-7.49 (m, 6 H) 10.83 (s, 1 H); MS (ESI, methanol/NH4OH) m/z 238 [M+H], 260 [M+Na], 236 [M−H].

### Example 13B

### methyl (1R,2R)-2-[(4′-{[anilino(cyanoimino)methyl]amino}-1,1′-biphenyl-4-yl)carbonyl]cyclopentanecarboxylate

Example 1I (20 mg, 62 μmol), Example 13A (15 mg, 62 μmol), and acetonitrile (0.4 mL) were combined and heated in the Emrys Optimizer at 140° C. for 20 minutes thrice. The mixture was combined with ethyl acetate (40 mL), washed with 1N aqueous NaOH (1×40 mL), washed with brine (1×40 mL), dried (Na₂SO₄), filtered, and concentrated. Purification by flash chromatography (elating with a gradient of 0 to 100%, ethyl acetate in hexane) provided the title compound as a yellow solid. ¹H NMR (300 MHz, DMSO-d₆) δ ppm 1.56-1.70 (m, 2 H) 1.73-1.87 (m, 2 H) 2.03 (m, 1 H) 2.16-2.24 (m, 1 H) 3.21-3.27 (m, 1 H) 3.57 (s, 3 H) 4.07-4.16 (m, 1 H) 7.14 (m, 1 H) 7.31-7.38 (m, 4 H) 7.44 (d, J=8.48 Hz, 2 H) 7.76 (d, J=8.48 Hz, 2 H) 7.83 (d, J=8.48 Hz, 2 H) 8.07 (d, J=8.48 Hz, 2 H) 9.59 (s, 2 H); MS (ESI, methanol/NH4OH) m/z 467 [M+H], 489 [M+Na], 465 [M−H].

### Example 13C

### (1R,2R)-2-[(4′-{[anilino(cyanoimino)methyl]amino}-1,2′-biphenyl-4-yl)carbonyl]cyclopentanecarboxylic acid

Example 13B (9 mg, 19 mmol) was dissolved in CH₂Cl₂ (15 mL) and cooled in an ice-water bath. Boron trichloride (58 μL, 58 μmol of a 1 M solution in CH₂Cl₂) was added in one portion. The reaction mixture was permitted to warm to room temperature as it was stirred overnight. After 22 hours the solution was diluted with ethyl acetate (40 mL) and 1 M aqueous HCl (40 mL). The layers were separated, and the organic layer was washed with brine (1×40 mL), dried (Na₂SO₄), filtered, and concentrated to a yellow residue. Purification by column chromatography (eluting first with 100% ethyl acetate then with 1:9 CH₃OH:CH₂Cl₂) gave the title compound as a white solid ¹H NMR (300 MHz, DMSO-d₆) δ ppm 1.63-1.78 (m, 2 H) 1.83 (m, 1 H) 1.94-2.03 (m, 2 H) 2.15-2.26 (m, 1H) 3.09-3.18 (m, 1 H) 4.13 (m, 1 H) 7.10-7.16 (m, 1 H) 7.30-7.39 (m, 4 H) 7.44 (d, J=8.48 Hz, 2 H) 7.77 (m, 4 H) 8.01 (d, J=8.48 Hz, 2 H) 9.60 (s, 1 H) 9.63 (s, 1 H) 11.87 (s, 1 H); MS ESI, methanol/NH4OH) m/z 453 [M+H], 475 [M+Na], 451 [M−H].

### Example 14

### N-(4′-{[(1R,2R)-2-cyanocyclopentyl]carbonyl}-1,1′-biphenyl-4-yl)-N′-phenylurea

### Example 14A

### methyl (1R,2R)-2-({4′-[(anilinocarbonyl)amino]-1,1′-biphenyl-4-yl}carbonyl)cyclopentanecarboxylate

Example 1I (0.9 g, 2.78 mmol) and phenyl isocyanate (0.36 g, 3.06 mmol) were placed in tetrahydrofuran (20 mL) at rt, stirred overnight, concentrated and purified by flash chromatography (5%-50% ethyl acetate in Hexane) to provide the title product. ¹ H NMR (500 MHz, DMSO-d₆) δ ppm 1.56-1.86 (m, 4H), 2.00-2.07 (m, 1H), 2.16-2.22 (m, 1H), 3.30-3.35 (m, 1H), 3.58 (s, 3H), 4.08-4.14 (m, 1H), 6.98 (t, J=7.4 Hz, 1H), 7.29 (t, J=7.7 Hz, 2H), 7.47 (d, J=7.7 Hz, 2H), 7.60 (d, J=8.9 Hz, 2H), 7.72 (d, J=8.9 Hz, 2H), 7.82 (d, J=8.6 Hz, 2H), 8.05 (d, J=8.6 Hz, 2H), 8.71 (s, 1H), 8.85 (s, 1H); MS (ESI) m/z 443.2 [M+H]⁺

### Example 14B

### (1R,2R)-2-({4′-[(anilinocarbonyl)amino]-1,1′-biphenyl-4-yl}carbonyl)cyclopentane carboxylic acid

Example 14A (0.8 g, 1.81 mmol), LiOH (0.38 g, 9 mmol) was stirred at room temperature overnight in 4:1 tetrahydrofuran/water mixture (50 mL). It was then adjusted to pH<7 with 4 M HCl. The mixture was concentrated and purified by RP-HPLC (Preparative reversed-phase chromatography was performed using a Zorbax SB-C18 7 μM 21.2×250 mm column with UV detection analyzed at 220 and 254 nM. Preparative method: (water with 0.1% trifluoroacetic acid and CH₃CN with 0.1% trifluoroacetic acid gradient) 5-95% CH₃CN over 30 minutes at 15 mL /min.) to provide the title product. ¹H NMR (500 MHz, DMSO-d₆) δ ppm 1.58-1.86 (m, 4H), 1.98-2.05 (m, 1H), 2.14-2.21 (m, 1H), 3.20-3.24 (m, 1H), 4.07-4.12(m, 1H), 6.99 (t, J=7.3 Hz, 1H), 7.29(t, J=8.3 Hz, 2H), 7.48 (d, J=7.6 Hz, 2H), 7.60 (d, J=8.5 Hz, 2H), 7.72 (d, J=8.5 Hz, 2H), 7.82 (d, J=8.2 Hz, 2H), 8.06 (d, J=8.5 Hz, 2H), 8.73 (s, 1H), 8.87 (s, 1H), 12.23(s, 1H); MS (ESI) m/z 429.1 [M+H]⁺.

### Example 14C

### (1R,2R)-2-({4′-[(anilinocarbonyl)amino]-1,1′-biphenyl-4-yl}carbonyl)cyclopentanecarboxamide

Example 14B (0.12 g, 0.28 mmol) was treated with N-hydroxy succinamide (0.065 g, 0.56 mmol), N-(3-dimethylaminopropyl)-N′-ethylcarbodiimide hydrochloride (0.107 g, 0.56 mmol) and N-methyl morpholine (0.16 mL, 1.12 mmol) in dichloromethane (5 mL) and stirred at rt for 2 h. The solvents were removed on a rotary evaporator and then diluted with a 1:1 mixture of ethyl acetate and water (20 mL). The organic layers were separated, washed with brine, dried (MgSO₄) and concentrated to a white solid. The residue was taken up in dioxane (5 mL) and ammonium hydroxide (0.2 mL, 1.4 mmol) was added to the solution at room temperature and stirred for 1 h. The reaction mixture was filtered and the filtrate concentrated to a white solid that was purified by RP-HPLC (Preparative reversed-phase chromatography was performed using a Zorbax SB-C18 7 μM 21.2×250 mm column with UV detection analyzed at 220 and 254 nM. Preparative method: (water with 0.1% trifluoroacetic acid and CH₃CN with 0.1% trifluoroacetic acid gradient) 5-95% CH₃CN over 30 minutes at 15 mL/min.) to afford the title compound. ¹H NMR (500 MHz, DMSO-d₆) δ ppm 1.56-1.79 (m, 4H), 1.98-2.04 (m, 1H), 2.07-2.11 (m, 1H), 3.01-3.06 (m, 1H), 4.07-4.13 (m, 1H), 6.78 (s, 1H), 6.98 (t, 1H, 7.36 Hz), 7.27-7.31 (m, 3H), 7.47 (d, J=7.67 Hz, 2H), 7.59 (d, J=8.90 Hz, 2H), 7.71 (d, J=8.90 Hz, 2H), 7.80 (d, J=8.59 Hz, 2H), 8.02 (d, J=8.59 Hz, 2H), 8.72 (s, 1H), 8.86 (s, 1H); MS (ESI) m/z 428.1 [M+H]⁺.

### Example 14D

### N-(4′-{[(1R,2R)-2-cyanocyclopentyl]carbonyl}-1,1′-biphenyl-4-yl)-N′-phenylurea

Trifluoroacetic anhydride (0.062 mL, 0.45 mmol) was added drop wise to a solution of Example 14C (0.038 g, 0.09 mmol) in pyridine (1.5 mL) maintained at −20° C. The reaction mixture was allowed to warm up to 0° C., quenched with water and extracted with ethyl acetate. The organic extracts were washed with water, brine, dried (MgSO₄) and purified by RP-HPLC (Preparative reversed-phase chromatography was performed using a Zorbax SB-C18 7 uM 21.2×250 mm column with UV detection analyzed at 220 and 254 nM Preparative method: (water with 0.1% trifluoroacetic acid and CH₃CN with 0.1% trifluoroacetic acid gradient) 5-95% CH₃CN over 30 minutes at 15 mL/min.) to afford the titled compound. ¹H NMR (499 MHz, DMSO-d₆) δ ppm 1.56-1.71 (m, 2H), 1.79-1.86 (m, 1H), 1.87-1.95 (m, 1H), 2.14-2.20 (m, 1H), 2.26-2.33 (m, 1H), 3.42 (q, J=8.2 Hz, 1H), 4.23-4.28 (m, 1H), 6.99 (t, J=7.3, 1H), 7.30 (t, J=7.6 Hz, 2H), 7.47 (d, J=7.6 Hz, 2H), 7.61 (d, J=8.8 Hz, 2H), 7.74 (d, J=8.8 Hz, 2H), 7.84 (d, J=8.5 Hz, 2H), 8.09 (d, J=8.5 Hz, 2H), 8.76 (s, 1H), 8.90 (s, 1H); MS (ESI) m/z 410.1 [M+H]⁺.

### Example 15

### trans-2-{4-[4-({[(3-chlorophenyl)amino]carbonyl}amino)cyclohexyl]benzoyl}cyclopentanecarboxylic acid

### Example 15A

### N-(3-chlorophenyl)-N′-(4-phenylcyclohexyl)urea

A mixture of 4-phenylcyclohexanone (4 g, 23 mmol), ammonium acetate (18 g, 230 mmol), and sodium cyanoborohydride (1 g, 16 mmol) in methanol (150 mL) was stirred at room temperature under N₂ for 16 h. The reaction mixture was filtered through celite, and the filtrate was concentrated to dryness, and then dissolved in tetrahydrofuran (50 mL). To this mixture, 3-chlorophenyl isocyanate (3 g, 20 mmol) was added, stirred at room temperature for 3 hours. The reaction was quenched by adding water and product was extracted with ethyl acetate The organic layer was washed with brine, and dried over Na₂SO₄. Purification by flash chromatography (ethyl acetate/Hexane, 1/10) afforded the titled compound as white solid. ¹H NMR (500 MHz, DMSO-d₆) δ ppm 1.6-1.70 (m, 6H), 1.72-1.81 (m, 2H), 2.66 (m, 1H), 3.94 (m, 1H), 6.57 (m, 1H), 6.94 (m, 1H), 7.13-7.33 (m, 7H), 7.71 (d, J=2.14 Hz, 1H), 8.54 (s, 1H); MS (DCI/NH₃) m/z 329 [M+H]⁺.

### Example 15B

### methyl cis-2-{4-[4-({[(3-chlorophenyl)amino]carbonyl}amino)cyclohexyl]benzoyl}cyclopentanecarboxylate

Step A

A solution of Example 1C (1 g, 5.81 mmol), SOCl₂ (1.3 mL, 17.8 mmol), and N,N-dimethylformamide (0.01 mL) in 10 mL CH₂Cl₂ was stirred at room temperature overnight under N₂. The solvent was removed by rotary evaporation at <40° C., and the residue was dried in vacuo for 1 hour.

Step B

A round bottom flask was charged with 15A (100 mg, 0.30 mmol), the intermediate from step A (114 mg, 0.60 mmol), aluminum chloride (360 mg, 2.7 mmol), and dichloroethane (5 mL). The reaction mixture was heated to 100° C. for 1 hour under N₂, and the solvent was removed by distillation The reaction mixture was then slowly poured into 50 mL of ice water, and then 100 mL of ethyl acetate was added. After the mixture was stirred for 10 minutes, the aqueous layer was separated, and extracted with 50 mL of ethyl acetate the combined organic layers were washed with water (2×50 mL) and saturated NaHCO₃ solution (50 mL), dried over Na₂SO₄, and filtered. Removal of solvent provided crude Example 15B that was further purified by using RP-HPLC (Preparative reversed-phase chromatography was performed using a Zorbax SB-C18 7 μM 21.2×250 mm column with UV detection analyzed at 220 and 254 nM. Preparative method: (water with 0.1% trifluoroacetic acid and CH₃CN with 0.1% trfluoroacetic acid gradient) 5-95% CH₃CN over 30 minutes at 15 mL/min). ¹H NMR (500 MHz, DMSO-d₆) δ ppm 1.6-2.2 (m, 14H), 2.69 (m, 1H), 3 27 (m, 1H), 3.66 (s, 3H), 3.94 (m, 1H), 4.07 (m, 1H), 6.59 (d, J=7.63 Hz, 1H), 6.93 (m, 1H), 7.15 (m, 1H), 7.25 (m, 1H), 7.45 (d, J=8.24 Hz, 2H), 7.71 (m, 1H), 7.96 (d, J=8.24 Hz, 2H), 8.64 (s, 1H); MS (DCI/NH₃) m/z 483 [M+H]³⁰ .

### Example 15C

### trans-2-{4-[4-({[(3-chlorophenyl)amino]carbonyl}amino)cyclohexyl]benzoyl}cyclopentanecarboxylic acid

Example 15C was prepared using the procedure as described for Example 10, substituting Example 15B for Example 6D. ¹H NMR (500 MHz, DMSO-d₆) δ ppm 1.6-1.85 (m, 12H), 1.96-2.2 (m, 2 H), 2.67 (m, 1H), 3.21 (m, 1H), 3.94 (m, 1H), 4.05 (m, 1H), 6.59 (d, J=7.63 Hz, 1H), 6.93 (m, 1H), 7.15 (m, 1H), 7.25 (t, J=7.94 Hz, 1H), 7.44 (d, J=8.24 Hz, 2H), 7.71 (m, 1H), 7.97 (d, J=8.24 Hz, 2H), 8.53 (s, 1H), 12.20 (s, 1H); MS (DCI/NH₃) m/z 469 [M+H]⁺.

### Example 16

### methyl (1R,2R)-2-[(4′-{[1-(cyclohexylamino)-2-nitrovinyl]amino}-1,1′-biphenyl-4-yl)carbonyl]cyclopentanecarboxylate

To a 10 mL round bottom flask, intermediate 1I (30.0 mg 0.0930 mmol), 1,1-bis(methylthio)-nitroethylene (16.0 mg, 0.0968 mmol) and 1 mL of ethanol were added. The reaction mixture was heated to reflux for 48 hours. After this time, the reaction solution was allowed to cool to room temperature. Cyclohexylamine (50.0 μL, 0.465 mmol) was added via syringe, and the reaction mixture heated to 60° C. for 24 hours. After this time, the reaction solution was cooled to room temperature, and the solvent evaporated. The residue was purified via RP-HPLC (Preparative reversed-phase chromatography was performed using a Zorbax SB-C18 7 uM 21.2×250 mm column with UV detection analyzed at 220 and 254 nM. Preparative method: (water with 0.1% trifluoroacetic acid and CH₃CN with 0.1% trifluoroacetic acid gradient) 5-95% CH₃CN over 30 minutes at 15 mL/min.) to afford the title compound. ¹H NMR (400 MHz, DMSO-d₆) δ ppm 1.19-1.33 (m, 1 H), 134-1.46 (m, 4 H), 1.54-1.65 (m, 2 H), 1.65-1.74 (m, 3 H), 1.74-1.80 (m, 2 H), 1.79-1.87 (m, 1 H), 1.92-2.10 (m, 3 H), 2.14-2.25 (m, 1 H), 3.27-3.37 (m, 1 H), 3.58 (s, 3 H), 3.76-3.83 (m, 1 H), 4.07-4.17 (m, 1 H), 6.18 (s, 1 H), 7.39 (d, -J=8.59 Hz, 2 H), 7.87 (dd, J=14 27, 8 44 Hz, 4 H), 8.09 (d, J=8.59 Hz, 2 H); MS (ESI) m/z 492.2 {M+H}⁺.

### Example 17

### methyl (1R,2R)-2-(4-{6-[(anilinocarbonyl-3-yl]benzoyl)cyclopentanecarboxylate

### Example 17A

### (1R,2R)-2-[4-(6-Amino-pyridin-3-yl)-benzoyl]-cyclopentanecarboxylic acid methyl ester

2-Amino-5-(4,4,5,5-tetramethyl-1,3,2-dioxaborolan-2-yl)pyridine (0.11 g, 0.5 mmol) was added to a suspension of Example 1G (0.1 g, 0.32 mmol), potassium fluoride (0.06 g, 1 mmol), tetrakis(triphenylphosphine)palladium(0) (0.037 g, 0.032 mmol) in a solvent mixture (10:1:6:3:dimethoxyethane:toluene:ethanol, H₂O, 10 mL) and degassed with nitrogen for 10 minutes. The reaction mixture was then heated at 90° C. for 12 hours, cooled, quenched with water (20 mL), extracted with ethyl acetate. The organic extracts were then washed with water, brine, dried (MgSO₄), filtered, concentrated and purified by flash chromatography using ethyl acetate as eluent to afford the title compound. ¹H NMR (499 MHz, DMSO-d₆) δ ppm 1.55-1.69 (m, 2H), 1.72-1.87 (m, 2H), 1.99-2.06 (m, 1H), 2.15-2.22 (m, 1H), 3.57 (s, 3H), 4.09 (q, J=9.1 Hz, 1H), 6.25 (s, 2H), 6.55 (d, J=8.2, 1H), 7.54-7.57 (m, 1H), 7.60-7.65 (m, 2H), 7.75 (d, J=8.5 Hz, 2H), 7.81 (dd, J=24, 8.5 Hz, 1H), 8.01 (d, J=8.5 Hz, 2H), 8.37 (d, J=2.4 Hz, 1H).

### Example 17B

### Methyl (1R,2R)-2-(4-{6-[(anilinocarbonyl)amino]pyridin-3-yl}benzoyl)cyclopentanecarboxylate

Example 17A (0.06 g, 0.18 mmol) was placed with phenyl isocyanate (0.03 mL, 0.24 mmol) in tetrahydrofuran (6 mL) and stirred at room temperature for 12 hours. The reaction mixture was quenched with water, extracted with ethyl acetate, the organic extracts washed with water, brine, dried (MgSO₄), filtered and concentrated to a white solid. The crude solid was taken up in ethyl acetate and the resultant slurry filtered. The filtrate was purified by flash chromatography eluting with 50% ethyl acetate/hexanes to afford the title compound. ¹H NMR (400 MHz, DMSO-d₆) δ ppm 1.59-1.69 (m, 2H), 1.72-1.89 (m, 2H), 1.99-2.06 (m, 1H), 2.15-2.22 (m, 1H), 3.33 (q, J=8.2 Hz, 1H), 3.58 (s, 3H), 4.13 (q, J=7.7 Hz, 1H), 7.04 (t, J=7.4 Hz, 1H), 7.33 (t, J=8.3, 2H) 7.54 (d, J=7.7 Hz, 2H), 7.70 (d, J=8.9 Hz, 1H), 7.89 (d, J=8.6 Hz, 2H), 8.09 (d, J=8.6 Hz, 2H), 8.19 (dd, J=2.5, 8.6 Hz, 1H), 8.73 (d, J=2.5 Hz, 1H), 9.58 (s, 1H), 10.29 (s, 1H); MS (ESI) m/z 444.2 [M+H]⁺.

### Example 18

### (1R,2R)-2-(4-{6-[(anilinocarbonyl)amino]pyridin-3-yl]benzoyl)cyclopentanecarboxylic acid

Example 17B (0.04 g, 0 09 mmol) was placed with lithium hydroxide monohydrate (0.02 g, 0.3 -mmol) in tetrahydrofuran (4 mL) and water (1 ml) and stirred at room temperature for 2 hours. The reaction mixture was then acidified with 3N HCl, extracted with ethyl acetate, organic extracts washed with water and brine, dried (MgSO₄), filtered and concentrated. The solid residue was taken up in ethyl acetate and the slurry was filtered to isolate the residue as the titled compound. ¹H NMR (500 MHz, DMSO-d₆) δ ppm 1.57-1.70 (m, 2H), 1.72-1.87 (m, 2H), 1.99-2.06 (m, 1H), 2.15-2.22 (m, 1H), 3.23 (q, J=8.2 Hz, 1H), 4.11 (q, J=8.8 Hz, 1H), 7.04 (t, J=7.3 Hz, 1H), 7.33 (t, J=7.6, 2H), 7.54 (d, J=7.6 Hz, 2H), 7.70 (d, J=8.8 Hz, 1H), 7.89 (d, J=8.6 Hz, 2H), 8.11 (d, J=8.6 Hz, 2H), 8.19 (dd, J=2.5, 8.6 Hz, 1H) 8.73 (d, J=2.5 Hz, 1H), 9.60 (s, 1H), 10.31 (s, 1H), 12.25 (broad s, 1H); MS (ESI) m/z 430.1 [M+H]⁺.

### Example 19

### methyl (1R,2R)-2-{[4′-({[(3-chlorophenyl)amino]carbonothioyl}amino)-1,1′-biphenyl-4-yl]carbonyl}cyclopentanecarboxylate

Example 19 was prepared using the procedure as described for Example 1J substituting 3-chlorophenyl isothiocyanate for 3-chlorophenyl isocyanate. ¹H NMR (500 MHz, DMSO-d₆) δ ppm 1.56-1.88 (m, 4H), 1.99-2.08 (m, 1H), 2.16-2.23 (m, 1H), 3.29-3.35 (m, 1H), 3.58 (s, 3H), 4.09-4.15 (m, 1H), 7.18 (d, J=7.98 Hz, 1H), 7.36 (t, J=7.98 Hz, 1H), 7.43 (d, J=8.29 Hz, 1H), 7.64 (d, J=8.60 Hz, 2H), 7.72-7.77 (m, 3H), 7.84 (d, J=8.29 Hz, 2H), 8.07(d, J=8.28 Hz, 2H), 9.99 (s, 2H), 10.09 (s, 1H); MS (ESI) m/z 493.1 [M+H]⁺.

### Example 20

### (1R,2R)-2-{[4′-({[(3-chlorophenyl)amino]carbonothioyl}amino)-1,1′-biphenyl-4-yl]carbonyl}cyclopentanecarboxylic acid

Example 20 was prepared using the procedure as described for Example 10, substituting Example 19 for Example 6D. ¹H NMR (500 MHz, DMSO-d₆) δ ppm 1.56-1.87 (m, 4H), 1.97-2.07 (m, 1H), 2.14-2.20 (m, 1H), 3.20-3.25 (m, 1H), 4.07-4.13 (m, 1H), 7.19 (d, J=7.67 Hz, 1H), 7.36 (t, J=7.98 Hz, 1H), 7.43 (d, J=8.60 Hz, 1H), 7.64 (d, J=8.59 Hz, 2H), 7.72-7.77 (m, 3H), 7.84 (d, J=8.59 Hz, 2H), 8.08 (d, J=8.29 Hz, 2H), 9.99 (s, 2H), 10.09 (s, 1H), 12.19 (br s, 1H); MS (ESI) m/z 479.1 [M+H]⁺.

### Example 21

### Trans-2-[(5-{4-[(anilinocarbonyl)amino]phenyl}pyridin-2-yl)carbonyl]cyclopentanecarboxylic acid

### Example 21A

### 2-[(5-bromopyridin-2-yl)carbonyl]cyclopentanecarboxylic acid

Step One:

1-Ethyl-3-[3-(dimethylamino)propyl]-carbodiimide hydrochloride (8.01 g, 41.82 mmol) and 1-hydroxybenzotriazole hydrate (5.65 g, 41.82 mmol) were sequentially added to a solution of 2-(methoxycarbonyl)cyclopentanecarboxylic acid (6.0 g, 34.85 mmol), N,O-dimethylhydroxylamine hydrochloride (3.74 g, 38.33 mmol) and N-methyl morpholine (11.5 mL, 104.55 mmol) in N,N-dimethylformamide (60 mL) maintained at room temperature. After overnight stirring, the reaction was quenched with water (100 mL) and extracted with ethyl acetate (4×150 mL). The organic extracts were washed with water (5×100 mL), brine, dried (MgSO₄), filtered, and concentrated to an oil and used as is in step two.

Step Two:

Lithium hydroxide monohydrate (1.95 g, 47 mmol) was added to a solution of the crude product (4 g, 18.6 mmol) from step one in tetrahydrofuran (45 mL) and water (15 mL) and the mixture stirred at room temperature for 14 hours. The reaction was quenched by the addition of 3N hydrochloric acid (reaction adjusted to pH 1) and extracted with ethyl acetate (4×150 mL). The organic extracts were washed with water, brine, dried (MgSO₄), filtered, and concentrated to an oil and used as is in step three.

Step Three:

Iso-Propyl magnesium bromide (2M solution in tetrahydrofuran, 33 mL, 66 mmol) was added drop wise via an addition funnel to a solution of 5-bromo-2-iodo-pyridine (17 g, 60 mmol) in tetrahydrofuran (100 mL) maintained at −20° C. The reaction was allowed to warm up to 5° C. over 2 hours. The reaction was cooled to −15° C., a solution of the crude product from step two (3.0 g, 15 mmol) in tetrahydrofuran (25 mL) was added drop wise to the mixture, and the mixture warmed up to 0° C. over 2 hours. The reaction was then quenched by addition of saturated aqueous ammonium chloride solution, adjusted to pH 1 with 3N hydrochloric acid, and extracted with ethyl acetate (5×150 mL). The organic extracts were washed with water, brine, dried (MgSO₄), filtered, concentrated to an oil and purified by flash chromatography using 10-75% ethyl acetate/hexanes as the eluent to afford the title compound. ¹H NMR (300 MHz, DMSO d₆) δ ppm 1.55-1.75 (m, 3 H), 1.81-1.95 (m, 3 H), 3.31-3.37 (m, 1H), 4.14-4.22 (m, 1 H), 7.84 (d, J=6 Hz, 1H), 8.24 (dd, J1=6 Hz, J2=3 Hz, 1H), 8.84 (d, J=3 Hz, 1 H), 11.95 (broad s, 1 H); MS (ESI) m/z 297.8 [M+H]⁺.

### Example 21B

### Methyl 2-[(5-bromopyridin-2-yl)carbonyl]cyclopentanecarboxylate

(Trimethylsilyl)diazomethane (2 M solution in hexanes, 4.6 mL, 9.23 mmol) was added drop wise to a solution of Example 21A (2.3 g, 7.69 mmol) in benzene (50 mL) and methanol (18 mL) and the mixture stirred at room temperature for 30 minutes. The reaction was quenched by addition of glacial acetic acid and then concentrated in vacuo. The residue was purified by flash chromatography using 8-30% ethyl acetate/hexanes as the eluent to afford the title compound. ¹H NMR (500 MHz, DMSO d₆) δ ppm 1.61-1.65 (m, 2 H), 1.67-1.73 (m, 1 H), 1.80-1.85 (m, 1 H), 1.91-2.02 (m, 1 H), 2.10-2.15 (m, 1 H), 3.21 (q, J=8.54 Hz, 1 H), 3.55 (s, 3 H), 4.31-4.40 (m, 1 H), 7.94 (dd, J1=8.54 Hz, J2=0.61 Hz, 1 H), 8.28 (dd, J=8.54 Hz, J2=2.44 Hz, 1 H), 8.88 (dd, J1=2.44 Hz, J2=0.61 Hz, 1 H); MS (ESI) m/z 313.9 [M+H]⁺.

### Example 21C

### Trans-methyl 2-{[5-(4-nitrophenyl)pyridin-2-yl]carbonyl}cyclopentanecarboxylate

A solution of Example 21B (0.5 g, 1.6 mmol), 4-nitrophenylboronic acid pinacol ester (0.52 g, 2.1 mmol), potassium fluoride (0.28 g, 4.83 mmol) and palladium-tetrakis(triphenylphosphine) (0.19 g, 0.16 mmol) in a solvent mixture of 1,2-dimethoxyethane:ethanol:water:toluene (10:6:3:1, 50 mL) was degassed with nitrogen for 10 minutes and then heated at 90° C. for 15 hours. The reaction was cooled to room temperature, quenched with water (50 mL) and extracted with ethyl acetate (4×100 mL). The organic extracts were washed with water, brine, dried (MgSO₄), filtered, concentrated to an oil and purified by flash chromatography using 20% ethyl acetate/hexanes as the eluent to afford the title compound. ¹H NMR (500 MHz, DMSO d₆) δ ppm 1.63-1.70 (m, 2 H), 1.73-1.79 (m, 1 H), 1.80-1.88 (m, 1 H), 2.02-2.09 (m, 1 H), 2.15-2.21 (m, 1 H), 3.25 (q, J=8.24 Hz, 1 H), 3.57 (s, 3 H), 4.46 (q, J=9.15 Hz, 1 H), 8.12-8.15 (m, 3 H), 8.38 (d, J=8.85 Hz, 2 H), 8.45 (dd, J1=8.24 Hz, J2=2.14 Hz, 1 H), 9.17 (d, J=2.44 Hz, 1 H).

### Example 21D

### Trans-methyl 2-{[5-(4-aminophenyl)pyridin-2-yl]carbonyl}cyclopentanecarboxylate

A suspension of Example 21C (0.37 g, 1.06 mmol), iron powder (0.12 g, 2.12 mmol) and ammonium chloride (0.07 g, 1.27 mmol) in ethanol (15 mL) and water (5 mL) were heated at 90° C. for 15 hours. The reaction was cooled to room temperature, and diluted with water and ethyl acetate. It was then filtered through a pad of wet celite and the organic layers were separated and washed with water and brine, dried (MgSO₄), filtered, and concentrated to afford the title compound. ¹H NMR (500 MHz, DMSO d₆) δ ppm 1.63-1.70 (m, 2 H), 1.73-1.79 (m, 1 H), 1.80-1.88 (m, 1 H), 2.02-2.09 (m, 1 H), 2.15-2.21 (m, 1 H), 3.25 (q, J=8.24 Hz, 1 H), 3.57 (s, 3 H), 4.46 (q, J=9.15 Hz, 1 H), 8.12-8.15 (m, 3 H), 8.38 (d, J=8.85 Hz, 2 H), 8.45 (dd, J1=8.24 Hz, J2=2.14 Hz, 1 H), 9.17 (d, J=2.44 Hz, 1 H).

### Example 21E

### Trans-2-[(5-{4-[(anilinocarbonyl)amino]phenyl}pyridin-2-yl)carbonyl]cyclopentanecarboxylic acid

Step One

Phenyl isocyanate (0.01 mL, 0.07 mmol) was added to a solution of Example 21D (0.02 g, 0.06 mmol) in tetrahydrofuran (2 mL) and the mixture stirred at room temperature for 15 hours. The reaction was quenched with water, extracted with ethyl acetate, concentrated and purified by reverse phase high pressure liquid chromatography (RP-HPLC) using a Zorbax SB-C18 7M 21.2×250 mm column with UV detection analyzed at 220 and 254 nM, and eluted with a solvent system containing component A (water with 0.1% trifluoroacetic acid) and component B (acetonitrile with 0.1% trifluoroacetic acid) with gradient of 5-95% of component B over 30 minutes at 15 mL/min to isolate the ester.

Step Two

Lithium hydroxide (0.02 g) was added to a solution of the ester from step one in tetrahydrofuran (2 mL) and water (1 mL) and the mixture stirred at room temperature for 10 hours. The reaction was quenched with 3N hydrochloric acid, extracted with ethyl acetate, dried (MgSO₄), filtered, concentrated and purified by RP-HPLC (using a Zorbax SB-C18 7M 21.2×250 mm column with UV detection analyzed at 220 and 254 nM, and eluted with a solvent system containing component A (water with 0.1% trifluoroacetic acid) and component B (acetonitrile with 0.1% trifluoroacetic acid) with gradient of 5-95% of component B over 30 minutes at 15 mL/min) to afford the title compound ¹H NMR (500 MHz, DMSO d₆) δ ppm 1.55-1.62 (m, 1 H), 1.63-1.70 (m 1H) 1.73-1.79 (m, 1 H), 1 H), 1.80-1.88 (m, 1 H), 1.99-2.06 (m, 1 H), 2.14-2.21 (m, 1 H), 3.22 (q, J=8.54 Hz, 1 H), 4.48 (q, J=8.85 Hz, 1 H), 6.99 (t, J=7.63 Hz, 1 H), 7.30 (t, J=7.32 Hz, 2 H), 7.47 (d, J=9.46 Hz, 2 H), 7.64 (d, J=8.54 Hz, 2 H), 7.80 (d, J=8.85 Hz, 2 H), 8.04 (d, J=8.54 Hz, 1 H), 8.25 (dd, J1=8.24 Hz, J2=2.45 Hz, 1 H), 8.76 (s, 1 H), 8.93 (s, 1 H), 9.06 (d, J=1.83 Hz, 1 H), 12.15 (broad s, 1 H); MS (ESI) m/z 430.2 [M+H]⁺.

### Example 22

### Trans-2-[(5-{4-[({[3-(trifluoromethyl)phenyl]amino}carbonyl)amino]phenyl}pyridin-2-yl)carbonyl]cyclopentanecarboxylic acid

The title compound was prepared as described in Example 21E, substituting 3-trifluoromethylphenyl isocyanate for phenyl isocyanate. ¹H NMR (500 MHz, DMSO d₆) δ ppm 1.55-1.62 (m, 1 H), 1.63-1.70 (m, 1H), 1.73-1.79 (m, 1 H), 1.80-1.88 (m, 1 H, 1.99-2.06 (m, 1 H), 2.14-2.21 (m, 1 H), 3.22 (q, J=8.54 Hz, 1 H), 4.48 (q, J=8.85 Hz, 1 H), 7.30-7.36 (m, 1 H), 7.50-7.56 (m, 1 L), 7.59-7.63 (m, 1 H), 7.65 (d, J=8.85 Hz, 2 H), 7.80 (d, J=8.85 Hz, 2 H), 8.03 (s, 1 H), 8.05 (dd, J1=8.24 Hz, J2=2.14 Hz, 1H), 8.27 (dd, J1=8.24 Hz, J2=2.45 Hz, 1 H), 9.08 (broad s,l I ), 9 17 (broad s, 1 H), 9.06 (dd, J1=2.45 Hz, J2=1.83 Hz, 1 H), 12.11 (broad s, 1 H); MS (ESI) m/z 498.2 [M+H]⁺.

### Example 23

### Trans-2-({5-[4-({[(3-chlorophenyl)amino]carbonyl}amino)phenyl]pyridin-2-yl}carbonyl)cyclopentanecarboxylic acid

The title compound was prepared as described in Example 21E substituting 3-chlorophenyl isocyanate for phenyl isocyanate. ¹H NMR (500 MHz, DMSO d₆) δ ppm 1.55-1.62 (m, 1 H), 1.63-1.70 (m, 1 H), 1.73-1.79 (m, 1 H), 1.80-1.88 (m, 1 H), 1.99-2.06 (m, 1 H), 2.14-2.21 (m, 1 H), 3.22 (q, J=8.54 Hz, 1 H), 4.48 (q, J=8.85 Hz, 1 H), 7.03-7.05 (m, 1 H), 7.28-7.34 (m, 2 H), 7.64 (d, J=8.55 Hz, 2 H), 7.73 (m, 1 H), 7.82 (d, J=8.85 Hz, 2 H), 8.04 (d, J=8.24 Hz, 1 H), 8.27 (dd, J1=8.24 Hz, J2=2.45 Hz, 1 H), 9.00 (s, 1 H), 9.03 (s, 1 H), 9.06 (d, J=1.83 Hz, 1 H), 12.15 (broad s, 1 H); MS (ESI) m/z 464.2 [M+H]⁺.

### Example 24

### Trans-2-({5-[4-({[(2-fluorophenyl)amino]carbonyl}amino)phenyl]pyridin-2-yl}carbonyl)cyclopentanecarboxylic acid

The title compound was prepared as described in Example 21E substituting 2-fluorophenyl isocyanate for phenyl isocyanate ¹H NMR (500 MHz, DMSO d₆) δ ppm 1.55-1.62 (m, 1 H), 1.63-1.70 (m, 1H), 1.73-1.79 (m, 1 H), 1.80-1.88 (m, 1 H), 1.99-2.06 (m, 1 H), 2.14-2.21 (m, 1 H), 3.22 (q, J=8.54 Hz, 1 H), 4.48 (q, J=8.85 Hz, 1 H), 7.01-7.05 (m, 1 H) 7.16 (d, J=7.93 Hz, 1 H), 7.26 (d, J1=7.93 Hz, J2=1.53 Hz, 1 H), 7.64 (d, J=8.85 Hz, 2 H), 7.80 (d, J=8.85 Hz, 2 H), 8.05 (d, J=8.24 Hz, 1 H), 8.16 (dt, J1=8.24 Hz, J2=1.53 Hz, 1 H), 8.27 (dd, J1=8.24 Hz, J2=2.14 Hz, 1 H), 8.63 (d, J=2.45 Hz, 1 H), 9.06 (d, J=2.13 Hz, 1 H), 9.30 (s, 1 H), 12.15 (broad s, 1 H); MS (ESI) m/z 448.2 [M+N]⁺.

### Example 25

### Trans-2-[(5-{4-[({[2-fluoro-5-(trifluoromethyl)phenyl]amino}carbonyl)amino]phenyl}pyridin-2-yl)carbonyl]cyclopentanecarboxylic acid

The title compound was prepared as described in Example 21E substituting 2-fluoro-5-trifluoromethylphenyl isocyanate. ¹H NMR (500 MHz, DMSO d₆) δ ppm 1.55-1.62 (m, 1 H), 1.63-1.70 (m, 1H), 1.73-1.79 (m, 1 H), 1.80-1.88 (m, 1 H), 1.99-2.06 (m, 1 H), 2.14-2.21 (m, 1 H), 3.22 (q, J=8.54 Hz, 1 H), 4.48 (q, J=8.85 Hz, 1 H), 7.40-7.43 (m, 1 H), 7.49-7.53 (m, 1 H), 7.64 (d, J=8.85 Hz, 2 H), 7.82 (d, J=8.85 Hz, 2 H), 8.06 (d, J=8.24 Hz, 1 H), 8.27 (dd, J1=8.24 Hz, J2=2.14 Hz, 1 H), 8.62 (dd, J1=7.33 Hz, J2=2.14 Hz, 1 H), 8.98 (d, J=2.75 Hz, 1 H), 9.07 (d, J=2.14 Hz, 1 H), 9.42 (s, 1 H), 12.15 (broad s, 1 H); MS (ESI) m/z 516.3 [M+H]⁺.

### Example 26

### Trans-2-[(5-{4-[(phenylacetyl)amino]phenyl}pyridin-2-yl)carbonyl]cyclopentanecarboxylic acid

Step One:

Phenyl acetic acid (0.01 g, 0.07 mmol) was added to a solution of Example 21D (0.02 g, 0.06 mmol), 1-ethyl-3-[3-(dimethylamino)propyl]-carbodiimide hydrochloride (0.015 g, 0.08 mmol) and 1 hydroxybenzotriazole hydrate (0.01 g, 0.08 mmol) in N,N-dimethylformamide (2 mL) and the mixture stirred at room temperature for 15 hours. The reaction was quenched with water, extracted with ethyl acetate, concentrated and purified by reverse phase high pressure liquid chromatography (RP-HPLC) using a Zorbax SB-C18 7M 21.2×250 mm column with UV detection analyzed at 220 and 254 nM, and eluted with a solvent system containing component A (water with 0.1% trifluoroacetic acid) and component B (acetonitrile with 0.1% trifluoroacetic acid) with gradient of 5-95% of component B over 30 minutes at 15 mL/min to isolate the ester.

Step Two

Lithium hydroxide (0.02 g) was added to a solution of the ester from step one in tetrahydrofuran (2 mL) and water (1 mL) and the mixture stirred at room temperature for 10 hours. The reaction was quenched with 3N hydrochloric acid, extracted with ethyl acetate, dried (MgSO₄), concentrated and purified by RP-HPLC (using a Zorbax SB-C18 7M 21.2×250 mm column with UV detection analyzed at 220 and 254 nM, and eluted with a solvent system containing component A (water with 0.1% trifluoroacetic acid) and component B (acetonitrile with 0.1% trifluoroacetic acid) with gradient of 5-95% of component B over 30 minutes at 15 mL/min) to afford the title compound. ¹H NMR (500 MHz, DMSO d₆) δ ppm 1.54-1.62 (m, 1 H), 1.63-1.70 (m, 1H), 1.73-1.79 (m, 1 H), 1.80-1.88 (m, 1 H), 1.93-2.06 (m, 1 H), 2.14-2.21 (m, 1 H) 3.20 (q, J=8.54 Hz, 1 H), 3.69 (s, 2 H), 4.48 (q, J=8.85 Hz, 1 H), 7.24-7.28 (m, 1 H), 7.32-7.36 (m, 4 H), 7.78 (d, J=8.84 Hz, 2 H), 7.81 (d, J=8.85 Hz, 2 H), 8.04 (d, J=8.24 Hz, 1 H), 8.26 (dd, J1=8.24 Hz, J2=2.14 Hz, 1 H), 9.05 (d, J=1.83 Hz, 1 H), 10.4 (s, 1 H), 12.11 (broad s, 1 H); MS (ESI) m/z 429.2 [M+H]⁺.

### Example 27

### Trans-2-{[5-(4-{[(2-ethoxyphenyl)acetyl]amino}phenylpyridin-2-yl]carbonyl}cyclopentanecarboxylic acid

The title compound was prepared as described in Example 26, substituting 2-ethoxyphenyl acetic acid for phenyl acetic acid ¹H NMR (500 MHz, DMSO d₆) δ ppm 1.26 (t, J=6.72 Hz, 3 H), 1.54-1.62 (m, 1 H), 1.63-1.70 (m, 1H), 1.73-1.79 (m, 1 H), 1.80-1.88 (m, 1 H), 1.93-2.06 (m, 1 H), 2.14-2.21 (m, 1 H), 3.20 (q, J=8.54 Hz, 1 H), 3.65 (s, 2 H), 4.00 (q, J=6.72 Hz, 2 H), 4.48 (q, J=8.85 Hz, 1 H), 6.86-6.91 (m, 1 H), 6.96 (d, J=8.24 Hz, 2 H), 7.78 (d, J=8.84 Hz, 2 H), 7.81 (d, J=8.85 Hz, 2 H), 8.04 (d, J=8.24 Hz, 1 H), 8.26 (dd, J1=8.24 Hz, J2=2.14 Hz, 1 H), 9.05 (d, J=1.83 Hz, 1 H), 10.27 (s, 1 H), 12.11 (broad s, 1 H); MS (ESI) m/z 473.2 [M+H]⁺.

### Example 28

### Trans-2-{[5-(4-{[(3,5-dimethylphenyl)acetyl]amino}phenyl)pyridin-2-yl]carbonyl}cyclopentanecarboxylic acid

The title compound was prepared as described in Example 26, substituting 3,5-dimethylphenyl acetic acid for phenyl acetic acid. ¹H NMR (500 MHz, DMSO d₆) δ ppm 1.54-1.62 (m, 1 H), 1.63-1.70 (m, 1H), 1.73-1.79 (m, 1 H), 1.80-1.88 (m, 1 H), 1.93-2.06 (m, 1 H), 2.14-2.21 (m, 1 H), 2.26 (s, 6 H), 3.20 (q, J=8.54 Hz, 1 H), 3.59 (s, 2 H), 4.48 (q, J=8.85 Hz, 1 H), 6.89 (s, 1 H), 6.96 (s, 2 H), 7.77 (d, J=8.84 Hz, 2 H), 7.81 (d, J=8.85 Hz, 2 H), 8.04 (d, J=8.24 Hz, 1 H), 8.26 (dd, J1=8.24 Hz, J2=2.14 Hz, 1 H), 9.05 (d, J=1.83 Hz, 1 H), 10.34 (s, 1 H), 12.11 (broad s, 1 H); MS (ESI) m/z 457.2 [M+H]⁺.

### Example 29

### Trans-2-{[5-(4-{[(2R)-2-phenylpropanoyl]amino}phenyl)pyridin-2-yl]carbonyl}cyclopentanecarboxylic acid

The title compound was prepared as described in Example 26, substituting (R)-(−)-2-phenyl propionic acid for phenyl acetic acid ¹H NMR (500 MHz, DMSO d₆) δ ppm 1.45 (d, J=6.72 Hz, 3 H), 1.51-1.62 (m, 1 H), 1.63-1.70 (m, 1 H) 1.73-1.79 (m, 1 H), 1.80-1.88 (m, 1 H), 1.93-2.06 (m, 1 H), 2.14-2.21 (m, 1 H) 3.20 (q, J=8.54 Hz, 1 H), 3.86 (q, J=6.72 Hz, 1 H), 4.48 (q, J=8.85 Hz, 1 H), 7.23-7.31 (m, 1 H), 7.33-7.36 (m, 2 H), 7.37-7.44 (m, 2 H), 7.77 (d, J=8.84 Hz, 2 H), 7.80 (d, J=8.85 Hz, 2 H), 8.04 (d, J=8.24 Hz, 1 H), 8.26 (dd, J1=8.24 Hz, J2 =2.14 Hz, 1 H), 9.04 (d, J=1.83 Hz, 1 H), 10.27 (s, 1 H), 12.14 (broad s, 1 H); MS (ESI) m/z 443.2 [M+H]⁺.

### Example 30

### Trans-2-{[5-(4-{[fluoro(phenyl)acetyl]amino}phenyl)pyridin-2-yl]carbonyl}cyclopentanecarboxylic acid

The title compound was prepared as described in Example 26, substituting α-fluorophenyl acetic acid for phenyl acetic acid ¹H NMR (500 MHz, DMSO d₆) δ ppm 1.54-1.62 (m, 1 H), 1.63-1.70 (m, 1H), 1.73-1.79 (m, 1 H), 1.80-1.88 (m, 1 H), 1.93-2.06 (m, 1 H), 2.14-2.21 (m, 1 H), 3.20 (q, J=8.54 Hz, 1 H), 4.48 (q, J=8.85 Hz, 1 H), 6.06 (s, 0.5 H, 6.15 (s, 0.5 H), 7.43-7.50 (m, 3 H), 7.52-7.63 (m, 2 H), 7.84 (d, J=8.84 Hz, 2 H), 7.87 (d, J=8.85 Hz, 2 H), 8.05 (d, J=8.24 Hz, 1 H), 8.28 (dd, J1=8.24 Hz, J2=2.14 Hz, 1 H), 9.06 (d, J=1.83 Hz, 1 H), 10.6 (s, 1 H), 12.13 (broad s, 1 H); MS (ESI) m/z 447.2 [M+H]⁺.

### Example 31

### Trans-2-[(5-{4-[(thien-3-ylacetyl)amino]phenyl}pyridin-2-yl)carbonyl]cyclopentanecarboxylic acid

The title compound was prepared as described in Example 26, substituting thiophene-3-acetic acid for phenyl acetic acid. ¹H NMR (500 MHz, DMSO d₆) δ ppm 1.54-1.62 (m, 1 H), 1.63-1.70 (m, 1H), 1.73-1.79 (m, 1 H), 1.80-1.88 (m, 1 H), 1.93-2.06 (m, 1 H), 2.14-2.21 (m, 1 H), 3.20 (q, J=8.54 Hz, 1 H), 3.70 (s, 2 H), 4.48 (q, J=8.85 Hz, 1 H), 7.12 (d, J=4.88 Hz, 1 H), 7.35 (d, J=2.14 Hz, 1 H), 7.50 (dd, J1=4.88 Hz, J2=2.14 Hz, 1 H), 7.78 (d, J=8.84 Hz, 2 H), 7.81 (d, J=8.85 Hz, 2 H), 8.05 (d, J=8.24 Hz, 1 H), 8.26 (dd, J1=8.24 Hz, J2=2.14 Hz, 1 H), 9.05 (d, J=1.83 Hz, 1 H), 10.36 (s, 1 H), 12.11 (broad s, 1 H); MS (ESI) m/z 435.1 [M+H]⁺.

### Example 32

### Trans-2-[(5-{4-[(pyridin-3-ylacetyl)amino]phenyl}pyridin-2-yl)carbonyl]cyclopentanecarboxylic acid

The title compound was prepared as described in Example 26, substituting pyridyl-3-acetic acid for phenyl acetic acid ¹H NMR (500 MHz, DMSO d₆) δ ppm 1.54-1.62 (m, 1 H), 1.63-1.70 (m, 1 H), 1.73-1.79 (m, 1 H), 1.80-1.88 (m, 1 H), 1.93-2.06 (m, 1 H), 2.14-2.21 (m, 1 H), 3.21 (q, J=8.54 Hz, 1 H), 3.92 (s, 2 H), 4.48 (q, J=8.85 Hz, 1 H), 7.72-7.78 (m, 3 H), 7.83 (d, J=8.84 Hz, 2 H), 8.05 (d, J=8.24 Hz, 1 H), 8.15-8.19 (m, 1 H), 8.26 (dd, J1=8.24 Hz, J2=2.14 Hz, 1 H), 9.05 (d, J=1.83 Hz, 1 H), 10.52 (s, 1 H), 12.11 (broad s, 1 H); MS (ESI) m/z 430.1 [M+H]⁺.

### Example 33

### Trans-2-{[5-(4-{[(1-phenylcyclopropyl)carbonyl]amino}phenyl)pyridin-2-yl]carbonyl}cyclopentanecarboxylic acid

The title compound was prepared as described in Example 26, substituting 1-phenyl-1-cyclopropane carboxylic acid for phenyl acetic acid. ¹H NMR (500 MHz, DMSO d₆) δ ppm 1.14-1.18 (m, 2 H), 1.47-1.49 (m, 2 H), 1.54-1.62 (m, 1 H), 1.63-1.70 (m, 1H), 1.73-1.79 (m, 1 H), 1.80-1.88 (m, 1 H), 1.93-2.06 (m, 1 H), 2.14-2.21 (m, 1 H), 3.20 (q, J=8.54 Hz, 1 H), 4.48 (q, J=8.85 Hz, 1 H), 7.28-7.31 (m, 1 H), 7.36-7.42 (m, 4 H), 7.74 (d, J=8.84 Hz, 2 H), 7.78 (d, J=8.85 Hz, 2 H), 8.04 (d, J=8.24 Hz, 1 H), 8.26 (dd, J1=8.24 Hz, J2=2.14 Hz, 1 H), 9.04 (d, J=1.83 Hz, 1 H), 9.32 (s, 1 H), 12.11 (broad s, 1 H); MS (ESI) m/z 455.2 [M+H]⁺.

### Example 34

### Trans-2-[(5-{4-[(anilinocarbonyl)amino]-3-fluorophenyl}pyridin-2-yl)carbonyl]cyclopentanecarboxylic acid

### Example 34A

### Trans-methyl 2-[(5-{4-[(tert-butoxycarbonyl)amino]-3-fluorophenyl}pyridin-2-yl)carbonyl]cyclopentanecarboxylate

The title compound was prepared as described in Example 21C, substituting 4-(tert-butoxycarbonylamino)-3-fluorophenylboronic acid for 4-nitrophenylboronic acid pinacol ester. ¹H NMR (500 MHz, DMSO d₆) δ ppm 1.49 (s, 9 H), 1.63-1.70 (m, 2 H), 1.73-1.79 (m, 1 H), 1.80-1.88 (m, 1 H), 1.97-2.05 (m, 1 H), 2.15-2.21 (m, 1 H), 3.25 (q, J=8.24 Hz, 1 H), 3.57 (s, 3 H), 4.48 (q, J=9.15 Hz, 1 H), 7.63-7.69 (m, 1 H), 7.75-7.85 (m, 2 H), 8.06 (d, J=8.54 Hz, 1 H), 8.28-8.33 (m, 1 H), 9.07 (dd, J1=13.15 Hz, J2=1.83 Hz, 1 H), 9.20 (broad s, 1 H); MS (ESI) m/z 443.1 [M+H]⁺.

### Example 34B

### Trans-methyl 2-{[5-(4-amino-3-fluorophenyl)pyridin-2-yl]carbonyl}cyclopentanecarboxylate

Example 34A was added to trifluoroacetic acid (7 mL) and dichloromethane (20 mL) and the mixture stirred at room temperature for 2 hours. The solvents were removed in vacuo, the residue was diluted with saturated sodium hydrogen carbonate, extracted with ethyl acetate, dried (MgSO₄), filtered, and concentrated to a brown paste, to afford the title compound, which was used in the next step without purification ¹H NMR (500 MHz, DMSO d₆) δ ppm 1.63-1.70 (m, 2 H), 1.73-1.79 (m, 1 H), 1.80-1.88 (m, 1 H), 1.97-2.05 (m, 1 H), 2.15-2.21 (m, 1 H), 3.22 (q, J=8.24 Hz, 1 H), 3.55 (s, 3 H), 4.46 (q, J=9.15 Hz, 1 H), 5.60 (s, 2 H), 6.91 (t, J=8.24 Hz, 1 H), 7.44 (dt, J1=7.93 Hz, J2=2.13 Hz, 1 H), 7.59 (dd, J1=13.15 Hz, J2=1.83 Hz, 1 H), 7.99 (d, J=8 24 Hz, 1 H), 8.19 (dt, J1=10.99 Hz, J2=2.13 Hz, 1 H), 8.99 (s, 1 H); MS (ESI) m/z 343.0 [M+H]⁺.

### Example 34C

### Trans-2[(5-{4[(anilinocarbonyl)amino]-3-fluorophenyl}pyridin-2-yl)carbonyl]cyclopentanecarboxylic acid

The title compound was prepared as described in Example 21E, substituting Example 34B for Example 21D. ¹H NMR (500 MHz, DMSO d₆) δ ppm 1.55-1.62 (m, 1 H), 1.63-1.70 (m, 1H), 1.73-1.79 (m, 1 H), 1.80-1.88 (m, 1 H), 1.99-2.06 (m, 1 H), 2.14-2.21 (m, 1 H), 3.22 (q, J=8.54 Hz, 1 H), 4.48 (q, J=8.85 Hz, 1 H), 7.01 (t, J=7.63 Hz, 1 H), 7.32 (t, J=7.63 Hz, 2 H), 7.47 (d, J=7 63 Hz, 2 H), 7.69 (dd, J1=8.54 Hz, J2=1.83 Hz, 1 H), 7.85 (dd, J1=12.51 Hz, J2=2.13 Hz, 1 H), 8.06 (d, J=8.23 Hz, 1 H), 8.31 (dd, J1=8.24 Hz, J2=2.13 Hz, 1 H), 8.36 (t, J=8,54 Hz, 1 H), 8.76 (d, J=2.45 Hz, 1 H), 9.10 (d, J=2.14 Hz, 1 H), 9.15 (s, 1 H), 12.15 (broad s, 1 H); MS (ESI) m/z 448.1 [M+H]⁺.

### Example 35

### Trans-2-[(5-{3-fluoro-4-[(phenylacetyl)amino]phenyl}pyridin-2-yl)carbonyl]cyclopentanecarboxylic acid

The title compound was prepared as described in Example 26, substituting Example 34B for Example 21D. ¹H NMR (500 MHz, DMSO d₆) δ ppm 1.55-1.62 (m, 1 H), 1.63-1.70 (m, 1H), 1.73-1.79 (m, 1 H), 1.80-1.88 (m, 1 H), 1.99-2.06 (m, 1 H), 2.14-2.21 (m, 1 H), 3.20 (q, J=8.54 Hz, 1 H), 3.78 (s, 2 H), 4.48 (q, J=8.85 Hz, 1 H), 7.24-7.30 (m, 1 H), 7.31-7.37 (m, 4 H), 7.67 (dd, J=854 Hz, J2=1.83 Hz, 1 H), 7.83 (dd, J1=12.51 Hz, J2=2.13 Hz, 1 H), 8.06 (d, J=8.23 Hz, 1 H), 8.08-8.13 (m, 1 H), 8.32 (dd, J1=8.24 Hz, J2=2.13 Hz, 1 H), 9.10 (d, J=2.14 Hz, 1 H), 10.15 (s, 1 H), 12.15 (broad s, 1 H); MS (ESI) m/z 447.2 [M+H]⁺

### Example 36

### Trans-2-({6′-[({[3-(trifluoromethyl)phenyl]amino}carbonyl)amino]-3,3′-bipyridin-6-yl]carbonyl)cyclopentanecarboxylic acid

### Example 36A

### Trans-methyl 2-[6′-amino-3,3′-bipyridin-6-yl)carbonyl]cyclopentanecarboxylate

The title compound was prepared as described in Example 21C, substituting 2-aminopyridine-5-boronic acid pinacol ester for 4-nitrophenylboronic acid pinacol ester. ¹H NMR (500 MHz, DMSO d₆) δ ppm 1.63-1.70 (m, 2 H), 1.73-1.79 (m, 1 H), 1.80-1.88 (m, 1 H), 2.02-2.09 (m, 1 H), 2.15-2.21 (m, 1 H), 3.22 (q, J=8.24 Hz, 1 H), 3.56 (s, 3 H), 4.44 (q, J=9.15 Hz, 1 H), 6.37 (s, 2 H), 6.58 (d, J=8.85 Hz, 1 H), 7.89 (dd, J1=8.54 Hz, J2=2.45 Hz, 1 H), 8.00 (d, J=8.54 Hz, 1 H), 8.19 (dd, J1=8.23 Hz, J2=2.45 Hz, 1 H), 8.45 (d, J=2.44 Hz, 1 H), 8.99 (d, J=1.53 Hz, 1 H).

### Example 36B

### Trans-2-(6′-(3-(3-(Trifluoromethyl)phenyl)ureido)-3,3′-bipyridine-6-carbonyl)cyclopentanecarboxylic acid

The title compound was prepared as described in Example 21E, substituting Example 36A for Example 21D, and substituting 3-trifluoromethylphenyl isocyanate for phenyl isocyanate. ¹H NMR (500 MHz, DMSO d₆) δ ppm 1.55-1.62 (m, 1 H), 1.63-1.70 (m, 1H), 1.73-1.79 (m, 1 H), 1.80-1.88 (m, 1 H), 1.99-2.06 (m, 1 H), 2.14-2.21 (m, 1 H), 3.22 (q, J=8.54 Hz, 1 H), 4.48 (q, J=8.85 Hz, 1 H), 7.38 (d, J=7.93 Hz, 1 H), 7.57 (t, J=8.24 Hz, 1 H), 7.68 (d, J=9.15 Hz, 1 H), 7.75 (d, J=8.85 Hz, 1 H), 8.07-8.11 (m, 2 H) 8.28 (dd, J1=8.85 Hz, J2=2.44 Hz, 1 H), 8.35 (dd, J1=8.85 Hz, J2=2.45 Hz, 1 H), 8.83 (d, J=2.14 Hz, 1 H), 9.12 (d, J=2.14 Hz, 1 H), 9.75 (s, 1 H), 10.56 (s, 1 H), 12.11 (broad s, 1 H); MS (ESI) m/z 499.2 [M+H)⁺.

### Example 37

### Trans-N-[2-fluoro-5-(trifluoromethyl)phenyl]-N′-[4-(2-{[2-(1-hydroxy-1-methylethyl)cyclopentyl]carbonyl}-1,3-thiazol-5-yl)phenyl]urea

### Example 37A

### {[tert-butyl(dimethyl)silyl]oxy}(1,3-thiazol-2-yl)acetonitrile

To a solution of thiazole-2-carbaldehyde (1.3 g, 11.5 mmol) in acetonitrile (38 mL) at ambient temperature, potassium cyanide (2.86 g, 44.0 mmol), tert-butylchlorodimethylsilane (2.08 g, 13.8 mmol), and zinc(II) iodide (0.055 g, 0.173 mmol) were added. After 16 hours, the reaction was filtered and concentrated under reduced pressure. The crude residue was purified by chromatography on silica gel, eluting with hexane, to afford the title compound. MS (ESI) m/z 255 [M+H]⁺.

### Example 37B

### Trans 2-(2-acetylcyclopentyl)-2-(tert-butyldimethylsilyloxy)-2-(thiazol-2-yl)acetonitrile

To a cold (−78 ° C.) solution of Example 37A (1.0 g, 3.92 mmol) in tetrahydrofuran (10 mL), lithium diisopropylamide (8.62 mL, 4.31 mmol, 0.5 M in tetrahydrofuran) was added over five minutes. After 30 minutes, 1-cyclopentenylethanone (0.476 mL, 4.31 mmol) was added drop wise. The reaction was stirred for an additional 30 minutes and was quenched by the addition of saturated aqueous ammonium chloride (20 mL) and ethyl acetate (20 mL). The layers were separated, and the aqueous was extracted with additional ethyl acetate (2×20 mL). The combined organic layers were dried with anhydrous sodium sulfate, filtered, and concentrated under reduced pressure. The crude residue was purified by chromatography on silica gel, eluting with a gradient of 5% ethyl acetate in hexane to 25% ethyl acetate in hexane, to afford the title compound. ¹H NMR (300 MHz, DMSO-d₆) δ ppm −0.15 (s, 3 H), 0.17 (s, 3 H), 1.46-1.68 (m, 4 H), 1.77 (s, 3 H), 1.75-1.82 (m, 1 H), 1.87-1.98 (m, 1 H), 2.59 (ddd, J=10.2, 6.8, and 6.8 Hz, 1 H), 2.69 (ddd, J=6.8, 6.8, and 3.4 Hz, 1 H), 7.72 (d, J=3.0 Hz, 1 H), 7.75 (d, J=3.0 Hz, 1H).

### Example 37C

### Trans {[tert-butyl(dimethyl)silyl]oxy}[2-(1-hydroxy-1-methylethyl)cyclopentyl]1,3-thiazol-2-ylacetonitrile

To a cold (0° C.) solution of Example 37B (0.682 g, 1.87 mmol) in tetrahydrofuran (9 mL), methylmagnesum bromide (1.67 mL, 2.34 mol, 1.4 M in tetrahydrofuran) was added. After 15 minutes, the reaction was quenched by the addition of saturated aqueous ammonium chloride (15 mL) and ethyl acetate (15 mL). The layers were separated, and the aqueous layer was extracted with additional ethyl acetate (2×15 mL). The combined organic layers were dried with anhydrous sodium sulfate, filtered, and concentrated under reduced pressure. The crude residue was purified by chromatography on silica gel, eluting with 5% ethyl acetate in hexane to 35% ethyl acetate in hexane, to afford the title compound. MS (ESI) m/z 381 [M+H]⁺.

### Example 37D

### Trans-(2R)-{[tert-butyl(dimethyl)silyl]oxy}[2-(1-hydroxy-1-methylethyl)cyclopentyl](5-iodo-1,3-thiazol-2-yl)acetonitrile

To a cold (−78° C.) solution of Example 36C (0.592 g, 56 mmol) in tetrahydrofuran (8 mL), n-butyllithium (1.38 mL, 3.43 mol, 2.48 M in hexane) was added After 15 minutes, a solution of iodine (0.476 g, 1.87 mmol) in tetrahydrofuran (2 mL) was added drop wise. After 5 minutes, the reaction was quenched by the addition of saturated aqueous ammonium chloride (20 mL) and ethyl acetate (20 mL). The layers were separated and the aqueous layer was extracted with additional ethyl acetate (2×20 mL). The combined organic layers were dried with anhydrous sodium sulfate, filtered, and concentrated under reduced pressure to afford the title compound, which was used in the subsequent step without further purification. MS (ESI) m/z 507 [M+H]⁺

### Example 37E

### Trans-[2-(1-hydroxy-1-methylethyl)cyclopentyl](5-iodo-1,3-thiazol-2-yl)methanone

To a solution of Example 37D (0.791 g, 1.56 mmol) at ambient temperature in a solvent mixture of tetrahydrofuran (5 mL) and acetic acid (0.5 mL), tetrabutyl ammonium fluoride (1.72 mL, 1.72 mmol, 1.0 M in tetrahydrofuran) was added. After 3 hours, the reaction was quenched by the addition of saturated aqueous sodium hydrogen carbonate (10 mL) and ethyl acetate (10 mL) The layers were separated, and the aqueous was extracted with additional ethyl acetate (2×10 mL). The combined organic layers were dried with anhydrous sodium sulfate, filtered, and concentrated under reduced pressure to afford the title compound, which was used in the subsequent step without further purification. MS (ESI) m/z 366 [M+H]⁺.

### Example 37F

### Trans-[2-(1-hydroxy-1-methylethyl)cyclopentyl][5-(4-nitrophenyl)-1,3-thiazol-2-yl]methanone

A solution of Example 37E (0.330 g, 0.904 mmol), 4-nitrophenylboronic acid (0.211 g, 2.70 mmol), potassium fluoride (0.157 g, 2.70 mmol), and bis(triphenylphosphine)palladium(II) dichloride (0.065 g, 0.090 mmol) in a mixture of 1,2-dimethoxyethane/methanol (1:1, 3 mL) was heated to 80° C. for 16 hours. The reaction was cooled to room temperature, and saturated aqueous ammonium chloride (5 mL) and ethyl acetate (5 mL) were added. The layers were separated, and the aqueous layer was extracted with additional ethyl acetate (2×5 mL). The combined organic layers were dried with anhydrous sodium sulfate, filtered, and concentrated under reduced pressure. The crude residue was purified by chromatography on silica gel, eluting with 50% ethyl acetate in hexane, to afford the title compound MS (ESI) m/z 361 [M+H]⁺

### Example 37G

### Trans-[5-(4-aminophenyl)-1,3-thiazol-2-yl][2-(1-hydroxy-1-methylethyl)cyclopentyl]methanone

To a solution of Example 37F (0.150 g, 0.417 mmol) and ammonium chloride (0.023 g, 0.417 mmol) in a solvent mixture of ethanol (4 mL) and water (1 mL) was added iron powder (0.056 g, 1.04 mmol). The mixture was heated at 80° C. for 8 hours. The reaction was then cooled to room temperature and quenched by the addition of saturated aqueous sodium hydrogen carbonate (10 mL) and ethyl acetate (10 mL). The layers were separated, and the aqueous was extracted with additional ethyl acetate (2×10 mL). The combined organic layers were dried with anhydrous sodium sulfate, filtered, and concentrated under reduced pressure to afford the title compound, which was used in the subsequent step without further purification. MS (ESI) m/z 331 [M+H]⁺.

### Example 37H

### Trans-N-[2-fluoro-5-(trifluoromethyl)phenyl]-N′-[4-(2-{[2-(1-hydroxy-1-methylethyl)cyclopentyl]carbonyl}-1,3-thiazol-5-yl)phenyl]urea

To a solution of Example 37G (0.123 g, 0.373 mmol) at ambient temperature in tetrahydrofuran (3 mL) was added 2-fluoro-5-trifluoromethylphenyl isocyanate (0.054 mL, 0.373 mmol). After 1 hour, the reaction was concentrated under reduced pressure. The crude residue was purified by chromatography on silica gel, eluting with ethyl acetate, to afford the title compound. ¹H NMR (300 MHz, DMSO-d₆) δ ppm 1.00 (s, 3 H), 1.07 (s, 3 H), 1.56-1.69 (m, 4 H), 1.75-1.80 (m, 1 H), 1.97-2.04 (m, 1 H), 2.53 (q, J=8.5 Hz, 1 H), 4.09 (q, J=8.5 Hz, 1 H), 4.21 (s, 1 H), 7.33 (d, J=7.6 Hz, 1 H), 7.53 (t, J=7.9 Hz, 1 H), 7.60 (d, J=8.9 Hz, 2 H), 7.75 (d, J=8.9 Hz, 2 H), 8.02 (s, 1 H), 8.45 (s, 1 H), 9.10 (s, 1 H), 9.13 (s, 1 H); MS (ESI) m/z 536 [M+H]⁺.

### Example 38

### Trans-2-[(5-{4-[({]2-fluoro-5-(trifluoromethyl)phenyl]amino}carbonyl)amino]phenyl}thien-2-yl)carbonyl]cyclobutane carboxylic acid

### Example 38A

### Cis-2-(methoxycarbonyl)cyclobutane carboxylic acid

A solution of 3-oxabicyclo[3.2.0]heptane-2,4-dione (1.65 g, 13.1 mmol) in methanol (20 mL) was heated to 60° C. for 16 hours. The reaction was then concentrated under reduced pressure to afford the title compound. MS (ESI) m/z 159 [M+H]⁺

### Example 38B

### Trans-methyl 2-[(5-bromothien-2-yl)carbonyl]cyclobutanecarboxylate

To solution of Example 38A (1.0 g, 6.5 mol) in a solvent mixture of dichloromethane (11 mL) and N,N-dimethylformamide (0.25 mL) at ambient temperature, thionyl chloride (1.44 mL, 19.7 mmol) was added. After 6 hours, the reaction was concentrated under reduced pressure. The residue was dissolved in 2-bromothiophene (3.8 mL, 39.0 mmol) and cooled to 0° C. Aluminum trichloride (1.73 g, 13 mmol) was added in a single portion with vigorous stirring. After 3 hours, the reaction was quenched by the slow addition of water (20 mL) and ethyl acetate (20 mL). The layers were separated, and the aqueous was extracted with additional ethyl acetate (2×10 mL). The combined organic layers were dried with anhydrous sodium sulfate, filtered, and concentrated under reduced pressure. The residue was purified by chromatography on silica gel, eluting with 10% ethyl acetate in hexane, to afford the title compound. MS (ESI) m/z 305 [M+H]⁺.

### Example 38C

### N-[2-fluoro-5-(trifluoromethyl)phenyl]-N′-[4-(4,4,5,5-tetramethyl-1,3,2-dioxaborolan-2-yl)phenyl]urea

To an ambient solution of 4-(4,4,5,5-tetramethyl-1,3,2-dioxaborolan-2-yl)aniline (2.0 g, 9.13 mmol) in tetrahydrofuran (30 mL), 2-fluoro-5-trifluoromethylphenyl isocyanate (1.32 mL, 9.13 mmol) was added. After 1 hour, the mixture was concentrated under reduced pressure to afford the title compound as a white solid. MS (ESI) m/z 425 [M+H]⁺.

### Example 38D

### Trans-2-[(5-{4-[({[2-fluoro-5-(trifluoromethyl)phenyl]amino}carbonyl)amino]phenyl}thien-2-yl)carbonyl]cyclobutane carboxylic acid

A solution of Example 38B (0.072 g, 0.236 mmol), Example 38C (0.100 g, 0.236 mmol), cesium fluoride (0.108 g, 0.0.708 mmol), and tetrakis(triphenylphosphine)palladium(0) (0.027 g, 0.024 mmol) in a solvent mixture of 1,2-dimethoxyethane (0.5 mL) and methanol (0.5 mL) was heated to 90° C. for 16 hours The reaction was then cooled to room temperature and diluted with ethyl acetate (5 mL) and water (5 mL). The layers were separated, and the aqueous was extracted with additional ethyl acetate (2×10 mL). The combined organic layers were dried with anhydrous sodium sulfate, filtered, and concentrated under reduced pressure to yield the intermediate ester. To the crude ester dissolved in tetrahydrofuran (0.6 mL) and methanol (0.3 mL) was added 2N sodium hydroxide (0.2 mL). The reaction was stirred at room temperature for 2 hours and was then acidified to pH 1 with 10% hydrochloric acid. After addition of ethyl acetate (5 mL), the layers were separated, and the aqueous was extracted with additional ethyl acetate (2×5 mL). The combined organic layers were dried with anhydrous sodium sulfate, filtered, and concentrated under reduced pressure. The residue was purified via RP-HPLC (preparative reversed-phase high pressure liquid chromatography) using a Zorbax SB-C18 7M 21.2×250 mm column with UV detection analyzed at 220 and 254 nM (preparative method: water with 0.1% trifluoroacetic acid and acetonitrile with 0.1% trifluoroacetic acid gradient 5-95% acetonitrile over 30 minutes at 15 mulminutes) to afford the title compound as a white solid. ¹H NMR (300 MHz, DMSO-d₆) δ ppm 2.06-2.28 (m, 4 H), 3.38-3.42 (m, 1 H), 4.17-4.25 (m, 1 H), 7.39-7.44 (m, 1 H), 7.50 (d, J=10.8 Hz, 1 H), 7.55-7 58 (m, 3 H), 7.74 (d, J=8.5 Hz, 1 H), 7.90 (d, J=4.1 Hz, 1 H), 8.61 (dd, J=7.1 and 1.7 Hz, 1 H), 8.96 (br s, 1 H), 9.42 (s, 1 H), 12.37 (s, 1 H); MS (ESI) m/z 507 [M+H]⁺.

### Example 39

### Trans-2-[(5-{4-[({[2-fluoro-5-(trifluoromethyl)phenyl]amino}carbonyl)amino]phenyl-1,3-thiazol-2-yl)carbonyl]cyclopentanecarboxylic acid

### Example 39A

### Trans-2-(1,3-thiazol-2-ylcarbonyl)cyclopentanecarboxylic acid

To a 20 mL vial with screw cap, tetrahydro-1H-cyclopenta[c]furan-1,3(3aH)-dione (1000 mg, 7.14 mmol) and tetrahydrofuran (8 mL) were added. Thiazol-2-ylzinc(II) bromide (17 mL, 8.5 mmol, 0.5 M in tetrahydrofuran) was added drop wise followed by tetrakis(triphenylphosphine)palladium(0) (400 mg, 0.36 mmol). The vial was sealed and the mixture stirred at 80° C. overnight. The reaction was cooled to room temperature and diluted with diethyl ether (25 mL) and 2.5M sodium hydroxide (25 mL). The layers were separated, and the aqueous layer was acidified to pH 1 with concentrated hydrochloric acid. The aqueous layer was extracted with diethyl ether (2×25 mL), and the combined organic layers were dried over sodium sulfate, filtered, and concentrated to afford the title compound. MS (ESI) m/z 226 [M+H]⁺.

### Example 39B

### Trans-methyl-2-(1,3-thiazol-2-ylcarbonyl)cyclopentanecarboxylate

To a 50 mL flask, Example 39A (1718 mg, 7.627 mmol), iodomethane (2.37 mL, 38.134 mmol), potassium carbonate (2100 mg, 15.253 mmol), and N,N-dimethylformamide (10 mL) were added. The solution was stirred at room temperature for 48 hours. The reaction was then diluted with diethyl ether (50 mL) and water (50 mL). The layers were separated, and the organic was washed with water (1×25 mL) and brine (1×25 mL), dried over sodium sulfate, filtered, and concentrated to afford the title compound. MS (ESI) m/z 240 [M+H]⁺.

### Example 39C

### Trans-methyl-2-{[5-(4,4,5,5-tetramethyl-1,3,2-dioxaborolan-2-yl)-1,3-thiazol-2-yl]carbonyl}cyclopentanecarboxylate

To a 25 mL flask, chloro-bis-cyclooctene-iridium (I) dimer (11 mg, 0.013 mmol), di-t-butylpyridine (7 mg, 0.025 mmol), 4,4,4′,4′,5,5,5′,5′-octamethyl-2,2′-bi(1,3,2-dioxaborolane (425 mg, 1.674 mmol), and octane (5 mL) were added. The reaction was stirred and heated to 50° C. for 30 minutes. Example 39B (200 mg, 0.837 mmol) in octane (5 mL) was added, and the solution was stirred at 80° C. for 16 hours. The reaction was cooled to room temperature and directly purified by chromatography on silica gel, eluting with 5% ethyl acetate in hexanes, to afford the title compound. MS (ESI) m/z 366 [M+H]⁺.

### Example 39D

### Trans-2-[(5-{4-[({[2-fluoro-5-(trifluoromethyl)phenyl]amino}carbonyl)amino]phenyl}-1,3-thiazol-2-yl)carbonyl]cyclopentanecarboxylic acid

To a 5 mL microwave reaction vessel, Example 39C (7 mg, 0.019 mmol), Example 44 (8 mg, 0.019 mmol), cesium fluoride (9 mg, 0.058 mmol), tetrakis(triphenylphosphine)palladium(0) (2 mg, 0.002 mmol), 1,2-dimethoxyethane (3 mL), and methanol(2 mL) were added. The reaction was heated to 150° C. for 5 minutes under microwave irradiation (Personal Chemistry Microwave) The solution was concentrated, taken up in methanol (2 mL) filtered through a plug of celite, and purified by RP-HPLC (Preparative reversed-phase high pressure liquid chromatography) using a Zorbax SB-C18 7M 21.2×250 mm column with UV detection analyzed at 220 and 254 nM (preparative method: water with 0.1% trifluoroacetic acid and acetonitrile with 0.1% trifluoroacetic acid gradient 5-95% acetonitrile over 30 minutes at 15 mL/minutes) to give an intermediate ester. The ester was dissolved in methanol (2 mL) and 2.5M sodium hydroxide (23 μL). The resulting solution was stirred at room temperature for 16 hours. The solution was acidified to pH 1 with 6N hydrochloric acid, and diluted with ethyl acetate (5 mL) and water (5 mL). The organic layer was washed with brine (1×5 mL), dried over anhydrous sodium sulfate, filtered, and concentrated under reduced pressure to afford the title compound. ¹H NMR (300 MHz, methanol-d₄) δ ppm 1.75-1.86 (m, 3 H), 1.88-2.01 (m, 1 H), 2.05-2.18 (m, 1 H), 2.24-2.34 (m, 1 H), 3.33-3.36 (m, 1 H), 4.26-4.34 (m, 1 H), 4.87 (s, 3 H), 7.29-7.38 (m, 2 H), 7.58-7.63 (m, 2 H), 7.68-7.74 (m, 2 H), 8.27 (s, 1 H), 8.61 (d, J=6.44 Hz, 1 H); MS (ESI) m/z 522 [M+H]⁺.

Example 40

### Trans-2-[(5-{3-fluoro-4-[({[3-(trifluoromethyl)phenyl]amino}carbonyl)amino]phenyl}-1,3-thiazol-2-yl)carbonyl]cyclopentanecarboxylic acid

The title compound was prepared as described in Example 39D, substituting Example 45 for 1-(2-fluoro-5-(trifluoromethyl)phenyl)-3-(4-iodophenyl)urea. ¹H NMR (300 MHz, methanol-d₄) δ ppm 1.76-1.86 (m, 3 H), 1.89-2.01 (m, 1 H), 2.12 (s, 1 H), 2.28 (s, 1 H), 3.34-3.43 (m, 1 H), 4.29 (s, 1 H), 4.76 (s, 3 H), 7.33 (s, 1 H), 7.46-7.49 (m, 1 H), 7.51-7.64 (m, 3 H), 7.94 (s, 1 H), 8.26-8.34 (m, 2 H); MS (ESI) m/z 522 [M+H]⁺

### Example 41

### Trans-2-[(5-{3-fluoro-4-[({[2-fluoro-5-(trifluoromethyl)phenyl]amino}carbonyl)amino]phenyl}-1,3-thiazol-2-yl)carbonyl]cyclopentanecarboxylic acid

The title compound was prepared as described in Example 39D, substituting Example 46 for 1-(2-fluoro-5-(trifluoromethyl)phenyl)-3-(4-iodophenyl)urea. ¹H NMR (300 MHz, methanol-d₄) δ ppm 1.76-1.87 (m, 3 H), 1.92-1.99 (m, 1 H), 2.08-2.16 (m, 1 H), 2.26-2.33 (m, 1 H), 3.21-3.27 (m, 1 H), 4.26-4.34 (m, 1 H), 4.73-4.75 (m, 3 H), 7.32-7.34 (m, 1 H), 7.35 (d, J=1.36 Hz, 1 H), 7.54-7.58 (m, 1 H), 7.61 (dd, J=11.87, 2.03 Hz, 1 H), 8.30 (s, 1 H), 8.36 (t, J=8.48 Hz, 1 H), 8.62-8.66 (m, 1 H); MS (ESI) m/z 540 [M+H]⁺

### Example 42

### Trans-2-[(5-{4-[({[2-fluoro-5-(trifluoromethyl)phenyl]amino}carbonyl)amino]phenyl}-1,3-thiazol-2-yl)carbonyl]cyclobutane carboxylic acid

### Example 42A

### Trans-2-(1,3-thiazol-2-ylcarbonyl)cyclobutane carboxylic acid

The title compound was prepared as described in Example 39A, substituting 3-oxabicyclo[3.2.0]heptane-2,4-dione for tetrahydro-1H-cyclopenta[c]furan-1,3(3aH)-dione. MS (APCI) m/z 212[M+H]⁺.

### Example 42B

### Trans-methyl-2-(1,3-thiazol-2-ylcarbonyl)cyclobutanecarboxylate

The title compound was prepared as described in Example 39B, substituting Example 42A for Example 39A. MS (APCI) m/z 226 [M+H]⁺.

### Example 42C

### Trans-methyl-2-{[5-(4,4,5,5-tetramethyl-1,3,2-dioxaborolan-2-yl)-1,3-thiazol-2-yl]carbonyl}cyclobutanecarboxylate

The title compound was prepared as described in Example 39C, substituting Example 42B for Example 39B. MS (ESI) m/z 352[M+H]⁺.

### Example 42D

### Trans-2-[(5-{4-[({[2-fluoro-5-(trifluoromethyl)phenyl]amino}carbonyl)amino]phenyl}-1,3-thiazol-2-yl)carbonyl]cyclobutane carboxylic acid

The title compound was prepared as described in Example 39D), substituting Example 42C for Example 39C ¹H NMR (300 MHz, methanol-d₄) δ ppm 2.19-2.29 (m, 2 H), 2.32-2.39 (m, 2 H), 3.53-3.63 (m, 1 H), 4.42-4.52 (m, 1 H), 4.87 (s, 3 H), 7.32 (t, J=8.82 Hz, 2 H), 7.55-7.62 (m, 2 H), 7.67-7.72 (m, 2 H), 8.24 (s, 1 H), 8.58-8.63 (m, 1 H); MS (ESI) m/z 508 [M+H]⁺.

### Example 43

### Trans-2-[(5-{3-fluoro-4-[({[3-(trifluoromethyl)phenyl]amino}carbonyl)amino]phenyl}thien-2-yl)carbonyl]cyclobutanecarboxylic acid

### Example 43A

### Trans-methyl 2-[(5-{4-[(tert-butoxycarbonyl)amino]-3-fluorophenyl}thien-2-yl)carbonyl]cyclobutanecarboxylate

To a 5 mL microwave reactor vial, 4-(tert-butoxycarbonylamino)-3-fluorophenylboronic acid (250 mg, 0.980 mmol), Example 38B (297 mg, 0.980 mmol), cesium fluoride (447 mg, 294 mmol), tetrakis(triphenylphosphine)palladium(0) (113 mg, 0.098 mmol), 1,2-dimethoxyethane (3 mL), and methanol (2 mL) were added. The reaction was heated to 150° C. for 5 minutes under microwave irradiation (Personal Chemistry Microwave). The reaction was concentrated, and the crude residue was purified by chromatography on silica gel, eluting with 15% ethyl acetate in hexanes, to afford the title compound. ¹H NMR (300 MHz, DMSO-d₆) δ ppm 1.46-1.51 (m, 9 H), 2.09-2.20 (m, 3 H), 2.22-2.30 (m, 1 H), 3.44-3.57 (m, 1 H), 3.59-3.62 (m, 3 H), 4.20-4.29 (m, 1 H), 7.54 (dd, J=8.31, 1.86 Hz, 1 H), 7.64-7.72 (m, 2 H), 7.76 (t, J=8.48 Hz, 1 H), 7.90 (d, J=4.07 Hz, 1 H), 9.21 (s, 1 H).

### Example 43B

### Trans-methyl-2-{[5-(4-amino-3-fluorophenyl)thien-2-yl]carbonyl}cyclobutanecarboxylate

To a 25 mL flask, Example 43A (210 mg, 0.485 mmol), dichloromethane (10 mL), and trifluoroacetic acid (2 mL) were added. The solution was stirred at room temperature for 3 hours, and washed with water (2×5 mL), saturated sodium hydrogen carbonate (2×5 mL), brine (1×5 mL), dried over sodium sulfate, filtered, and concentrated to afford the title compound. MS (APCI) m/z 434 [M+H]⁺.

### Example 43C

### Trans-methyl-2-[(5-{3-fluoro-4-[({[3-(trifluoromethyl)phenyl]amino}carbonyl)amino]phenyl}thien-2-yl)carbonyl]cyclobutanecarboxylate

To a 4 mL vial, Example 43B (160 mg, 0.48 mmol), 1-isocyanato-3-(trifluoromethyl)benzene (67 μL, 0.48 mmol), and tetrahydrofuran (2 mL) were added. The vial was sealed and the mixture stirred at 60° C. for 16 hours. The solution was concentrated to afford the title compound MS (APCI) m/z 521 [M+H]⁺

### Example 43D

### Trans-2-[(5-{3-fluoro-4-[({[3-trifluoromethyl)phenyl]amino}carbonyl)amino]phenyl}thien-2yl)carbonyl]cyclobutane carboxylic acid

To a 4 mL vial, Example 43C (150 mg, 0.288 mmol), methanol (2 mL), and 2.5M sodium hydroxide (0.35 mL) were added. The solution was stirred at room temperature for 16 hours. The solution was acidified to pH 1 with 6M hydrochloric acid, and diluted with ethyl acetate (5 mL) and water (5 mL). The layers were separated, and the organics were washed with brine (1×5 mL), dried over sodium sulfate, filtered, and concentrated under reduced pressure. The residue was purified by RP-HPLC (preparative reversed-phase high pressure liquid chromatography) using a Zorbax SB-C18 7M 21.2×250 mm column with UV detection analyzed at 220 and 254 nM (preparative method: water with 0.1% trifluoroacetic acid and acetonitrile with 0.1% trifluoroacetic acid gradient 5-95% acetonitrile over 30 minutes at 15 mL/minutes) to afford the title compound. ¹H NMR (300 MHz, methanol-d₄) δ ppm 2.19-2.34 (m, 4 H), 3.46-3.55 (m, 1 H), 4.20-4.29 (m, 1 H), 4.80-4.91 (m, 3 H), 7.27-7.33 (m, 1 H), 7.45 (d, J=4.07 Hz, 1 H), 7.48-7.62 (m, 4 H), 7.76-7.84 (m, 1 H), 7.94 (s, 1 H), 8.23-8.29 (m, 1 H); MS (ESI) m/z 507 [M+H]⁺.

### Example 44

### 1-(2-fluoro-5-(trifluoromethyl)phenyl)-3-(4-iodophenyl)urea

1-fluoro-2-isocyanato-4-(trifluoromethyl)benzene (2.00 grams, 9.75 mmol) was dissolved in 10 mL of tetrahydrofuran, and 4-iodoaniline (2.14 grams, 9.75 mmol) was added. The reaction vessel was heated to 65° C. for 3 hours. After this time, the reaction mixture was cooled to room temperature, and the solvent evaporated to afford the title compound.

### Example 45

### 1-(2-fluoro-4-iodophenyl)-3-(3-(trifluoromethyl)phenyl)urea

1-isocyanato-3-(trifluoromethyl)benzene (59.0 μL, 0.422 mmol) was dissolved in 4 mL of tetrahydrofuran, and 2-fluoro-4-iodoaniline (0.100 grams, 0.422 mmol) was added. The reaction vessel was heated to 65° C. for 3 hours. After this time, the reaction mixture was cooled to room temperature, and the solvent evaporated to afford the title compound.

### Example 46

### 1-(2-fluoro-4-iodophenyl)-3-(2-fluoro-5-(trifluoromethyl)phenyl)urea

1-fluoro-2-isocyanato-4-(trifluoromethyl)benzene (305 μL, 2.11 mmol) was dissolved in 5 mL of tetrahydrofuran, and 2-fluoro-4-iodoaniline (0.500 grams, 2.11 mmol) was added. The reaction vessel was heated to 65° C. for 3 hours. After this time, the reaction mixture was cooled to room temperature, and the solvent was evaporated to afford a solid that was triturated in boiling hexanes to afford the title compound.

It is understood that the foregoing detailed description and accompanying examples are merely illustrative and are not to be taken as limitations upon the scope of the invention, which is defined solely by the appended claims and their equivalents. Various changes and modifications including, but not limited to, those relating to the chemical structures, substituents, derivatives, intermediates, syntheses, formulations and/or methods of use of the invention, can be made without departing from the spirit and scope thereof.

What we claim is:

1. A compound of formula (I) 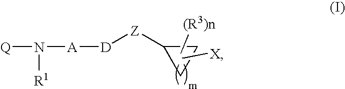
or a pharmaceutically acceptable salt, prodrug, salt of a prodrug, or a combination thereof, wherein
Q is —C(═Y)N(R²)(R^{2a}), —C(═W)(R^{b}), —R^{b}, —S(O)₂(R^{b}), or —C(O)O(R^{b});
R¹ and R^{2a}, are each independently hydrogen or lower alkyl;
R² is alkyl, aryl, heteroaryl, cycloalkyl, cycloalkenyl, or heterocycle; wherein the aryl, heteroaryl, cycloalkyl, cycloalkenyl, and heterocycle are each independently further unsubstituted or substituted with 1, 2, 3, 4, or 5 substituents independently selected from the group consisting of alkyl, alkenyl, alkynyl, nitro, —CN, halogen, ethylenedioxy, methylenedioxy, haloalkyl, —OR^{a}, —O—C(O)(R^{a}), —S(R^{a}), —S(O)(R^{b}), —S(O)₂(R^{b}), —C(O)(R^{a}), —C(O)O(R^{a}), —N(R^{a})₂, —N(R^{a})—C(O)(R^{a}), —C(O)N(R^{a})₂, —S(O)₂N(R^{a})₂, R⁴, —(CR^{c}R^{d})ₜ—OR^{a}, —(CR^{c}R^{d})ₜ—O—C(O)(R^{a}), —(CR^{c}R^{d})ₜ—S(R^{a}), —(CR^{c}R^{d})ₜ—S(O)(R^{b}), —(CR^{c}R^{d})ₜ—S(O)₂(R^{b}), —(CR^{c}R^{d})ₜ—C(O)(R^{a}), —(CR^{c}R^{d})ₜ—C(O)O(R^{a}), —(CR^{c}R^{d})ₜ—N(R^{a})₂, —(CR^{c}R^{d})ₜ—N(R^{a})—C(O)(R^{a}), —CR^{c}R^{d})ₜ—C(O)N(R^{a})₂, —(CR^{c}R^{d})ₜ—S(O)₂N(R^{a})₂, and —(CR^{c}R^{d})ₜ—R⁴;
R³ represents a substituent group selected from the group consisting of alkyl, haloalkyl, —OR^{a}, and halogen;
m is 1, 2, 3, 4, or 5;
n is 0, 1, or 2;
A and D are each a monocyclic ring selected from the group consisting of phenyl, heteroaryl, cycloalkyl, and cycloalkenyl; each of which is optionally further substituted with 1, 2, 3, 4, or 5 substituents as represented by T, wherein each T is independently selected from the group consisting of alkyl, alkenyl, alkynyl, nitro, —CN, halogen, ethylenedioxy, methylenedioxy, haloalkyl, —OR^{e}, —O—C(O)(R^{e}), —S(R^{e}), —S(O)(R^{f}), —S(O)₂(R^{f}), —C(O)(R^{e}), —C(O)O(R^{e}), —N(R^{e})₂, —(R^{e})—C(O)(R^{e}), —C(O)N(R^{e})₂, —S(O)₂N(R^{e})₂, —(CR^{c}R^{d})ₜ—ORₑ, —(CR^{c}R^{d})ₜ—O—C(O)(R^{e}), —(CR^{c}R^{d})ₜ—S(R^{e}), —(CR^{c}R^{d})ₜ—S(O)(R^{f}), —(CR^{c}R^{d})ₜ—S(O)₂(R^{f}), —(CR^{c}R^{d})ₜ—C(O)(R^{e}), —(CR^{c}R^{d})ₜ—C(O)O(R^{e}), —(CR^{c}R^{d})ₜ—N(R^{e})₂, —(CR^{c}R^{d})ₜ—N(R^{e})—C(O)(R^{e}), —(CR^{c}R^{d})ₜ—C(O)N(R^{e})₂, and —CR^{c}R^{d})ₜ—S(O)₂N(R^{e})₂; two adjacent substituents as represented by T, together with the carbon or nitrogen atom to which they are attached, optionally form a monocyclic ring selected from the group consisting of phenyl, heteroaryl, cycloalkyl and cycloalkenyl, and each of said monocyclic ring is independently further unsubstituted or substituted with 1, 2, 3, 4 or 5 substituents independently selected from the group consisting of alkyl, alkenyl, alkynyl, nitro, —CN, halogen, ethylenedioxy, methylenedioxy, haloalkyl, —OR^{e}, —O—C(O)(R^{e}), —S(R^{e}), —S(O)(R^{f}), —S(O)₂(R^{f}), —C(O)(R^{e}), —C(O)O(R^{e}), —N(R^{e})₂, —N(R^{e})—C(O)(R^{e}), —C(O)N(R^{e})₂, —S(O)₂N(R^{e})₂, —(CR^{c}R^{d})ₜ—OR^{e}, —(CR^{c}R^{d})ₜ—O—C(O)(R^{e}), —(CR^{c}R^{d})ₜ—S(R^{e}), —(CR^{c}R^{d})ₜ—S(O)(R^{f}), —(CR^{c}R^{d})ₜ—S(O)₂(R^{f}), —(CR^{c}R^{d})ₜ—C(O)(R^{e}), —(CR^{c}R^{d})ₜ—C(O)O(R^{e}), —CR^{c}R^{d})ₜ—N(R^{e})₂, —(CR^{c}R^{d})ₜ—N(R^{e})C(O)(R^{e}), —(CR^{c}R^{d})ₜ—C(O)N(R^{e})₂, and —(CR^{c}R^{d})ₜ—S(O)₂N(R^{e})₂;
Z is C(O), C(H)(OH), C(alkyl)(OH), O, N(R^{e}), S(O), S(O)₂, or CH₂;
Y is O, N(CN), S, or C(H)(NO₂);
W is O or S;
X represents a substituent group selected from the group consisting of —C(O)OR⁵, —C(O)N(R⁵)₂, —CN, —C(═NOR⁵)N(R⁵)₂, —C(R⁶R⁷)OH, —C(O)—N(R⁵)(OR⁵), and tetrazolyl; with the proviso that when Z is C(O) or C(H)(OH), A and D are phenyl, and X is located on the carbon atom that is adjacent to the carbon atom bearing Z, then X is —CN, —C(═NOR⁵)N(R⁵)₂, —C(R⁶R⁷)OH, —C(O)—N(R⁵)(OR⁵), or tetrazolyl; and with the further proviso that when Z is C(O), A is pyridinyl or pyrimidinyl, D is phenyl, and X is located on the carbon atom that is adjacent to the carbon atom bearing Z, then X is not —C(O)OH;
R⁵, at each occurrence, is independently hydrogen, alkyl, or haloalkyl;
R⁶ and R⁷ are independently hydrogen or alkyl, or R⁶ and R⁷ together with the carbon atom to which they are attached, form a three- to six-membered, monocyclic ring selected from the group consisting of cycloalkyl and cycloalkenyl;
R⁴, at each occurrence, is independently aryl, heteroaryl, cycloalkyl, cycloalkenyl, or heterocycle; wherein each R⁴ is independently unsubstituted or substituted with 1, 2, 3, 4 or 5 substituents independently selected from the group consisting of alkyl, alkenyl, alkynyl, nitro, —CN, halogen, ethylenedioxy, methylenedioxy, haloalkyl, —OR^{e}, —O—C(O)(R^{e}), —S(R^{e}), —S(O)(R^{f}), —S(O)₂(R^{f}), —C(O)(R^{e}), —C(O)O(R^{e}), —N(R^{e})₂, —N(R^{e})—C(O)(R^{e}), —C(O)N(R^{e})₂, —S(O)₂N(R^{e})₂, —(CR^{c}R^{d})ₜ—OR^{e}, —(CR^{c}R^{d})ₜ—O—C(O)(R^{e}), —(CR^{c}R^{d})ₜ—S(R^{e}), —CR^{c}R^{d})ₜ—S(O)(R^{f}), —(CR^{c}R^{d})ₜ—S(O)₂(R^{f}), —(CR^{c}R^{d})ₜ—C(O)(R^{e}), —(CR^{c}R^{d})ₜ—C(O)O(R^{e}), —(CR^{c}R^{d})ₜ—N(R^{e})₂, —(CR^{c}R^{d})ₜ—N(R^{e})—C(O)(R^{e}), —(CR^{c}R^{d})ₜ—C(o)N(R^{e})₂, and —(CR^{c}R^{d})ₜ—S(O)₂N(R^{e})₂;
R^{a}, at each occurrence, is independently hydrogen, alkyl, haloalkyl, R⁴, or —(CR^{g}R^{h})ᵤ—R⁴;
R^{b}, at each occurrence, is independently alkyl, haloalkyl, R⁴, or —(CR^{g}R^{h})ᵤ—R⁴;
R^{c}, R^{d}, R^{g}, and R^{h}, at each occurrence, are each independently hydrogen, halogen, alkyl or haloalkyl; or
R^{g} and R^{h}, together with the carbon atom to which they are attached, form a monocyclic, three- to six-membered cycloalkyl ring;
R^{e}, at each occurrence, is independently hydrogen, alkyl or haloalkyl;
R^{f}, at each occurrence, is independently alkyl or haloalkyl; and
u and t, at each occurrence, are each independently 1, 2, 3, or 4.

2. The compound of claim 1, or a pharmaceutically acceptable salt, prodrug, salt of a prodrug, or a combination thereof, wherein
Z is C(O) or C(H)OH;
X is —C(O)OR⁵ or —C(O)N(R⁵)₂, —CN, or —C(R⁶R⁷)OH; with the proviso that when X is located on the carbon atom adjacent to the carbon atom bearing Z, and A and D are phenyl, then X is —CN or —C(R⁶R⁷)OH; and with the further proviso that when X is located on the carbon atom adjacent to the carbon atom bearing Z, Z is C(O), A is pyrimidinyl or pyridinyl, and D is phenyl, then X is not —C(O)OH; and
R¹, R³, R⁵, R⁶, R⁷, Q, A, D, m, and n are as defined in claim 1.

3. The compound of claim 1, or a pharmaceutically acceptable salt, prodrug, salt of a prodrug, or a combination thereof, wherein
Z is C(O);
X is —C(O)OR⁵ or —C(O)N(R⁵)₂, with the proviso that when X is located on the carbon atom adjacent to the carbon atom bearing Z, then A and D are not both phenyl, and with the further proviso that when X is located on the carbon atom adjacent to the carbon atom bearing Z, A is pyrimidinyl or pyridinyl, and D is phenyl, then X is not —C(O)OH; and
R¹, R³, R⁵, Q, A, D, m, and n are as defined in claim 1.

4. The compound of claim 1, or a pharmaceutically acceptable salt, prodrug, salt of a prodrug, or a combination thereof, wherein
Z is C(O);
X is —C(O)OR⁵ or —C(O)N(R⁵)₂;
A is phenyl which is further unsubstituted or substituted with 1, 2, 3, 4, or 5 substituents T; and
D is monocyclic heteroaryl, which is further unsubstituted or substituted with 1, 2, 3, 4, or 5 substituents T.

5. The compound of claim 4, or a pharmaceutically acceptable salt, prodrug, salt of a prodrug, or a combination thereof, wherein Q is —C(═Y)N(R²)(R^{2a}).

6. The compound of claim 5 wherein X is —C(O)OH.

7. The compound of claim 1, or a pharmaceutically acceptable salt, prodrug, salt of a prodrug, or a combination thereof, wherein
Z is C(O);
X is —C(R⁶R⁷)OH, and
R¹, R³, R⁶, R⁷, Q, A, D, m and n are as defined in claim 1.

8. The compound of claim 7, or a pharmaceutically acceptable salt, prodrug, salt of a prodrug, or a combination thereof, wherein
A and D are phenyl, each of which is independently further unsubstituted or substituted with 1, 2, 3, 4, or 5 substituents T.

9. The compound of claim 7, or a pharmaceutically acceptable salt, prodrug, salt of a prodrug, or a combination thereof, wherein
A is phenyl which is further unsubstituted or substituted with 1, 2, 3, 4, or 5 substituents T; and
D is monocyclic heteroaryl that is further unsubstituted or substituted with 1, 2, 3, 4, or 5 substituents T.

10. The compound of claim 1, or a pharmaceutically acceptable salt, prodrug, salt of a prodrug, or a combination thereof, wherein
Z is C(H)(OH);
X is —C(R⁶R⁷)OH; and
R¹, R³, R⁶, R⁷, Q, A, D, m, and n are as defined in claim 1.

11. The compound of claim 1 comprising formula (Ia), 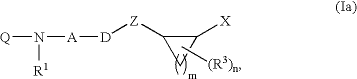
or a pharmaceutically acceptable salt, prodrug, salt of a prodrug, or a combination thereof, wherein R¹, R³, Q, A, D, Z, X, m, and n are as defined in claim 1.

12. The compound of claim 11, or a pharmaceutically acceptable salt, prodrug, salt of a prodrug, or a combination thereof, wherein
Z is C(O);
X is —C(O)OR⁵ or —C(O)N(R⁵)₂, with the proviso that A and D are not both phenyl, and with the further proviso that when A is pyrimidinyl or pyridinyl, and D is phenyl, then X is not —C(O)OH.

13. The compound of claim 11, or a pharmaceutically acceptable salt, prodrug, salt of a prodrug, or a combination thereof, wherein
Z is C(O);
X is —C(O)OR⁵ or —C(O)N(R⁵)₂;
A is phenyl which is further unsubstituted or substituted with 1, 2, 3, 4, or 5 substituents T; and
D is monocyclic heteroaryl that is further, unsubstituted or substituted with 1, 2, 3, 4, or 5 substituents T.

14. The compound of claim 1 comprising formula (If), 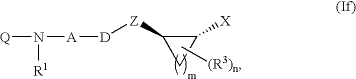
or a pharmaceutically acceptable salt, prodrug, salt of a prodrug, or a combination thereof, wherein R¹, R³, Q, A, D, Z, X, m, and n are as defined in claim 1.

15. The compound of claim 14, or a pharmaceutically acceptable salt, prodrug, salt of a prodrug, or a combination thereof, wherein
Z is C(O);
X is —C(O)OR⁵ or —C(O)N(R⁵)₂, with the proviso that A and D are not both phenyl, and with the further proviso that when A is pyrimidinyl or pyridinyl, and D is phenyl, then X is not —C(O)OH.

16. The compound of claim 14, or a pharmaceutically acceptable salt, prodrug, salt of a prodrug, or a combination thereof, wherein
Z is C(O);
X is —C(O)OR⁵ or —C(O)N(R⁵)₂;
A is phenyl which is further unsubstituted or substituted with 1, 2, 3, 4, or 5 substituents T; and
D is monocyclic heteroaryl, which is further unsubstituted or substituted with 1, 2, 3, 4, or 5 substituents T.

17. The compound of claim 1 selected from the group consisting of:
N-(3-chlorophenyl)-N′-(4′-{(R)-hydroxy[(1R,2R)-2-(hydroxymethyl)cyclopentyl]methyl}-1,1′-biphenyl-4-yl)urea;
N-(3-chlorophenyl)-N′-(4′-{(S)-hydroxy[(1R,2R)-2-(hydroxymethyl)cyclopentyl]methyl}-1,1′-biphenyl-4-yl)urea;
N-(3-chlorophenyl)-N′-(4′-{[(1R,2R)-2-(1-hydroxy-1-methylethyl)cyclopentyl]carbonyl}-1,1′-biphenyl-4-yl)urea;
N-(4′-{[(1R,2R)-2-(1-hydroxy-1-methylethyl)cyclopentyl]carbonyl}-1,1′-biphenyl-4-yl)-N′-phenylurea;
N-(4′-{(S)-hydroxy[(1R,2R)-2-(hydroxymethyl)cyclopentyl]methyl}-1,1′-biphenyl-4-yl)-N′-phenylurea;
N-(4′-{(R)-hydroxy[(1R,2R)-2-(hydroxymethyl)cyclopentyl]methyl}-1,1′-biphenyl-4-yl)-N′-phenylurea;
N-(4′-{[(1R,2R)-2-(hydroxymethyl)cyclopentyl]methyl}-1,1′-biphenyl-4-yl)-N′-phenylurea;
methyl (1R,2R)-2-{4-[5-({[(3-chlorophenyl)amino]carbonyl}amino)thien-2-yl]benzoyl}cyclopentanecarboxylate;
methyl (1R,2R)-2-(4-{5-[(anilinocarbonyl)amino]thien-2-yl}benzoyl)cyclopentanecarboxylate;
methyl (1R,2R)-2-(4-{5-[({[3-(trifluoromethyl)phenyl]amino}carbonyl)amino]thien-2-yl}benzoyl)cyclopentanecarboxylate;
(1R,2R)-2-{4-[5-({[(3-chlorophenyl)amino]carbonyl}amino)thien-2-yl]benzoyl}cyclopentanecarboxylic acid;
(1R,2R)-2-(4-{5-[(anilinocarbonyl)amino]thien-2-yl}benzoyl)cyclopentanecarboxylic acid;
(1R,2R)-2-(4-{5-[({[3-(trifluoromethyl)phenyl]amino}carbonyl)amino]thien-2-yl}benzoyl)cyclopentanecarboxylic acid;
N-(4′-{[(1R,2R)-2-cyanocyclopentyl]carbonyl}-1,1′-biphenyl-4-yl)-N′-phenylurea;
trans-2-{4-[4-({[(3-chlorophenyl)amino]carbonyl}amino)cyclohexyl]benzoyl}cyclopentanecarboxylic acid;
methyl (1R,2R)-2-(4-{6-[(anilinocarbonyl)amino]pyridin-3-yl}benzoyl)cyclopentanecarboxylate;
Trans-2-[(5-{4-[(anilinocarbonyl)amino]phenyl}pyridin-2-yl)carbonyl]cyclopentanecarboxylic acid;
Trans-2-[(5-{4-[({[3-(trifluoromethyl)phenyl]amino}carbonyl)amino]phenyl}pyridin-2-yl)carbonyl]cyclopentanecarboxylic acid;
Trans-2-({5-[4-({[(3-chlorophenyl)amino]carbonyl}amino)phenyl]pyridin-2-yl}carbonyl)cyclopentanecarboxylic acid;
Trans-2-({5-[4-({[(2-fluorophenyl)amino]carbonyl}amino)phenyl]pyridin-2-yl}carbonyl)cyclopentanecarboxylic acid;
Trans-2-[(5-{4-[({[2-fluoro-5-(trifluoromethyl)phenyl]amino]carbonyl)amino]phenyl}pyridin-2-yl)carbonyl]cyclopentanecarboxylic acid;
Trans-2-[(5-{4-[(phenylacetyl)amino]phenyl}pyridin-2-yl)carbonyl]cyclopentanecarboxylic acid;
Trans-2-{[5-(4-{[(2-ethoxyphenyl)acetyl]amino}phenyl)pyridin-2-yl]carbonyl}cyclopentanecarboxylic acid;
Trans-2-{[5-(4-{[(3,5-dimethylphenyl)acetyl]amino}phenyl)pyridin-2-yl]carbonyl}cyclopentanecarboxylic acid;
Trans-2-{[5-(4-{[(2R)-2-phenylpropanoyl]amino}phenyl)pyridin-2-yl]carbonyl}cyclopentanecarboxylic acid;
Trans-2-{[5-(4-{[fluoro(phenyl)acetyl]amino}phenyl)pyridin-2-yl]carbonyl}cyclopentanecarboxylic acid;
Trans-2-[(5-{4-[(thien-3-ylacetyl)amino]phenyl}pyridin-2-yl)carbonyl]cyclopentanecarboxylic acid;
Trans-2-[(5-{4-[(pyridin-3-ylacetyl)amino]phenyl}pyridin-2-yl)carbonyl]cyclopentanecarboxylic acid;
Trans-2-{[5-(4-{[(1-phenylcyclopropyl)carbonyl]amino}phenyl)pyridin-2-yl]carbonyl}cyclopentanecarboxylic acid;
Trans-2-[(5-{4-[(anilinocarbonyl)amino]-3-fluorophenyl}pyridin-2-yl)carbonyl]cyclopentanecarboxylic acid;
Trans-2-[(5-{3-fluoro-4-[(phenylacetyl)amino]phenyl}pyridin-2-yl)carbonyl]cyclopentanecarboxylic acid;
Trans-2-({6′-[({[3-(trifluoromethyl)phenyl]amino}carbonyl)amino]-3,3′-bipyridin-6-yl}carbonyl)cyclopentanecarboxylic acid;
Trans-N-[2-fluoro-5-(trifluoromethyl)phenyl]-N′-[4-(2-{[(1S,2S)-2-(1-hydroxy-1-methylethyl)cyclopentyl]carbonyl}-1,3-thiazol-5-yl)phenyl]urea;
Trans-2-[(5-{4-[({[2-fluoro-5-(trifluoromethyl)phenyl]amino}carbonyl)amino]phenyl}thien-2-yl)carbonyl]cyclobutane carboxylic acid;
Trans-2-[(5-{4-[({[2-fluoro-5-(trifluoromethyl)phenyl]amino}carbonyl)amino]phenyl}-1,3-thiazol-2-yl)carbonyl]cyclopentanecarboxylic acid;
Trans-2-[(5-{3-fluoro-4-[({[3-(trifluoromethyl)phenyl]amino}carbonyl)amino]phenyl}-1,3-thiazol -2-yl)carbonyl]cyclopentanecarboxylic acid;
Trans-2-[(5-{3-fluoro-4-[({[2-fluoro-5-(trifluoromethyl)phenyl]amino}carbonyl)amino]phenyl}-1,3-thiazol-2-yl)carbonyl]cyclopentanecarboxylic acid;
Trans-2-[(5-{4-[({[2-fluoro-5-(trifluoromethyl)phenyl]amino}carbonyl)amino]phenyl}-1,3-thiazol-2-yl)carbonylcyclobutane carboxylic acid; and
Trans-2-[(5-{3-fluoro-4-[({[3-(trifluoromethyl)phenyl]amino}carbonyl)amino]phenyl}thien-2-yl)carbonyl]cyclobutane carboxylic acid;
or a pharmaceutically acceptable salt, prodrug, or salt of a prodrug thereof.

18. A method for treating a disorder selected from the group consisting of type 2 diabetes, obesity, elevated plasma triglycerides, metabolic syndrome, non-alcoholic steatohepatitis, and non-alcoholic fatty liver disease comprising the step of administering to a subject in need thereof a compound of claim 1, or a pharmaceutically acceptable salt thereof.

19. The method of claim 18 further comprising the step of co-administering with one or more pharmaceutical agents selected from the group consisting of DPPIV inhibitor, incretin mimetic, metformin, fenofibrate, rimonabant, sibutramine, orlistat, statin, and nicotinic acid.

20. A pharmaceutical composition comprising a therapeutically effective amount of a compound of claim 1, or a pharmaceutically acceptable salt thereof, in combination with a pharmaceutically acceptable carrier

21. A pharmaceutical composition comprising a therapeutically effective amount of a compound of claim 1, or a pharmaceutically acceptable salt thereof, one or more pharmaceutical agents selected from the group consisting of DPPIV inhibitor, incretin mimetic, metformin, fenofibrate, rimonabant, sibutramine, orlistat, statin, and nicotinic acid, in combination with a pharmaceutically acceptable carrier.

22. A method of treating a disorder selected from the group consisting of type 2 diabetes, obesity, elevated plasma triglycerides, metabolic syndrome, non-alcoholic steatohepatitis, and non-alcoholic fatty liver disease comprising the step of administering to a subject in need thereof a pharmaceutical composition of claim 20.

23. A method for treating a disorder selected from the group consisting of type 2 diabetes, obesity, elevated plasma triglycerides, metabolic syndrome, non-alcoholic steatohepatitis, and non-alcoholic fatty liver disease comprising the step of administering to a subject in need thereof a pharmaceutical composition of claim 21.
